SEQUENCE LISTING (84 sequences)

SEQ ID NO: 1 - protein, 499 aa, Aspergillus oryzae
MMVAWWSLFLYGLQVAAPALAATPADWRSQSIYFLLTDRFARTDGSTTATCNTADRKYCGGTWQGIIDKLDYIQGMGFTAIWITPVTAQLPQTTAYGDAYHGYWQQDIYSLNENYGTADDLKALSSALHERGMYLMVDVVANHMGYDGAGSSVDYSVFKPFSSQDYFHPFCLIQNYEDQTQVEDCWLGDNTVSLPDLDTTKDVVKNEWYDWVGSLVSNYSIDGLRIDTVKHVQKDFWPGYNKAAGVYCIGEVLDGDPAYTCPYQNVMDGVLNYPIYYPLLNAFKSTSGSMDDLYNMINTVKSDCPDSTLLGTFVENHDNPRFASYTNDIALAKNVAAFIILNDGIPIIYAGQEQHYAGGNDPANREATWLSGYPTDSELYKLIASANAIRNYAISKDTGFVTYKNWPIYKDDTTIAMRKGTDGSQIVTILSNKGASGDSYTLSLSGAGYTAGQQLTEVIGCTTVTVGSDGNVPVPMAGGLPRVLYPTEKLAGSKICSSS

SEQ ID NO: 2 - protein, 719 aa, Geobacillus stearothermophilus
MKKKTLSLFVGLMLLIGLLFSGSLPYNPNAAEASSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 3 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 4 - protein, 551 aa, Pseudomonas saccharophila
MSHILRAAVLAAVLLPFPALADQAGKSPAGVRYHGGDEIILQGFHWNVVREAPNDWYNILRQQASTIAADGFSAIWMPVPWRDFSSWTDGGKSGGGEGYFWHDFNKNGRYGSDAQLRQAAGALGGAGVKVLYDVVPNHMNRGYPDKEINLPAGQGFWRNDCADPGNYPNDCDDGDRFIGGESDLNTGHPQIYGMFRDELANLRSGYGAGGFRFDFVRGYAPERVDSWMSDSADSSFCVGELWKGPSEYPSWDWRNTASWQQIIKDWSDRAKCPVFDFALKERMQNGSVADWKHGLNGNPDPRWREVAVTFVDNHDTGYSPGQNGGQHHWALQDGLIRQAYAYILTSPGTPVVYWSHMYDWGYGDFIRQLIQVRRTAGVRADSAISFHSGYSGLVATVSGSQQTLVVALNSDLANPGQVASGSFSEAVNASNGQVRVWRSGSGDGGGNDGGEGGLVNVNFRCDNGVTQMGDSVYAVGNVSQLGNWSPASAVRLTDTSSYPTWKGSIALPDGQNVEWKCLIRNEADATLVRQWQSGGNNQVQAAAGASTSGSF

SEQ ID NO: 5 - protein, 926 aa, Bacillus naganoensis
DGNTTNIVVHYFRPSGDYTDWNLWMWPENGDGAEYDFNQPTDSYGEVASVDIPGNPSQVGIIVRKGNWDAKDIDSDRYIDLSKGHEIWLVQGNSQIFYSEKDAEAAAQPAVSNAYLDASNQVLVKLSQPFTLGEGSSGFTVHDDTANKDIPVTSVSDANQVTAVLAGTFQHIFGGSDWAPDNHNTLLKKVNSNLYQFSGNLPEGNYQYKVALNDSWNNPSYPSDNINLTVPAGGAHVTFSYIPSTHAVYDTINNPNADLQVDSSGVKTDLVAVTLGENPDVSHTLSIQTEDYQAGQVIPRKVLDSSQYYYSGDDLGNTYTKNATTFKVWAPTSTQVNVLLYNSATGAVTKTVPMTASGHGVWEATVNQDLENWYYMYEVTGQGSTRTAVDPYATAIAPNGTRGMIVDLAKTDPAGWESDKHITPKNIEDEVIYEMDVRDFSIDSNSGMKNKGKYLALTEKGTKGPDNVKTGVDSLKQLGITHVQLQPVFAFNSVNENDPTQYNWGYDPRNYNVPEGQYATNANGTTRIKEFKEMVLSLHQDHIGVNMDVVYNHTFATQISDFDKIVPEYYYRTDDAGNYTNGSGTGNEIAAERPMVQKFIIDSLKFWVNEYHVDGFRFDLMALLGKDTMSKAATQLHAIDPGIALYGEPWTGGTSALPADQLLTKGAQKGMGVAVFNDNLRNGLDGSVFDSSAQGFATGATGLTDAIKNGVEGSINDFTASPGETINYVTSHDNYTLWDKIAQSNPNDSEADRIKMDELAQAIVMTSQGIPFMQGGEEMLRTKGGNDNSYNAGDVVNEFDWSRKAQYPDVFNYYSGLIHLRLDHPAFRMTTANEINSHLQFLNSPENTVAYELSDHANKDTWGNIVVIYNPNKTAETINLPSGKWEINATSGKVGESTLGQAEGSVQVPGISMMILHQEVSPSDGK

SEQ ID NO: 6 - protein, 829 aa, Bacillus acidopullulyticus
DSTSTEVIVHYHRFDSNYANWDLWMWPYQPVNGNGAAYEFSGKDDFGVKADVQVPGDDTQVGLIVRTNDWSQKNTSDDLHIDLTKGHEIWIVQGDPNIYYNLSDAQAAATPKVSNAYLDNEKTVLAKLTNPMTLSDGSSGFTVTDKTTGEQIPVTAATNANSASSSEQTDLVQLTLASAPDVSHTIQVGAAGYEAVNLIPRNVLNLPRYYYSGNDLGNVYSNKATAFRVWAPTASDVQLLLYNSETGPVTKQLEMQKSDNGTWKLKVPGNLKNWYYLYQVTVNGKTQTAVDPYVRAISVNATRGMIVDLEDTNPPGWKEDHQQTPANPVDEVIYEVHVRDFSIDANSGMKNKGKYLAFTEHGTKGPDNVKTGIDSLKELGINAVQLQPIEEFNSIDETQPNMYNWGYDPRNYNVPEGAYATTPEGTARITQLKQLIQSIHKDRIAINMDVVYNHTFNVGVSDFDKIVPQYYYRTDSAGNYTNGSGVGNEIATERPMVQKFVLDSVKYWVKEYHIDGFRFDLMALLGKDTMAKISKELHAINPGIVLYGEPWTGGTSGLSSDQLVTKGQQKGLGIGVFNDNIRNGLDGNVFDKSAQGFATGDPNQVNVIKNRVMGSISDFTSAPSETINYVTSHDNMTLWDKISASNPNDTQADRIKMDELAQAVVFTSQGVPFMQGGEEMLRTKGGNDNSYNAGDSVNQFDWSRKAQFENVFDYYSWLIHLRDNHPAFRMTTADQIKQNLTFLDSPTNTVAFELKNHANHDKWKNIIVMYNPNKTAQTLTLPSGNWTIVGLGNQVGEKSLGHVNGTVEVPALSTIILHQGTSEDVIDQN

SEQ ID NO: 7 - protein, 776 aa, Pseudomonas amyloderamosa
MKCPKILAALLGCAVLAGVPAMPAHAAINSMSLGASYDAQQANITFRVYSSQATRIVLYLYSAGYGVQESATYTLSPAGSGVWAVTVPVSSIKAAGITGAVYYGYRAWGPNWPYASNWGKGSQAGFVSDVDANGDRFNPNKLLLDPYAQEVSQDPLNPSNQNGNVFASGASYRTTDSGIYAPKGVVLVPSTQSTGTKPTRAQKDDVIYEVHVRGFTEQDTSIPAQYRGTYYGAGLKASYLASLGVTAVEFLPVQETQNDANDVVPNSDANQNYWGYMTENYFSPDRRYAYNKAAGGPTAEFQAMVQAFHNAGIKVYMDVVYNHTAEGGTWTSSDPTTATIYSWRGLDNATYYELTSGNQYFYDNTGIGANFNTYNTVAQNLIVDSLAYWANTMGVDGFRFDLASVLGNSCLNGAYTASAPNCPNGGYNFDAADSNVAINRILREFTVRPAAGGSGLDLFAEPWAIGGNSYQLGGFPQGWSEWNGLFRDSLRQAQNELGSMTIYVTQDANDFSGSSNLFQSSGRSPWNSINFIDVHDGMTLKDVYSCNGANNSQAWPYGPSDGGTSTNYSWDQGMSAGTGAAVDQRRAARTGMAFEMLSAGTPLMQGGDEYLRTLQCNNNAYNLDSSANWLTYSWTTDQSNFYTFAQRLIAFRKAHPALRPSSWYSGSQLTWYQPSGAVADSNYWNNTSNYAIAYAINGPSLGDSNSIYVAYNGWSSSVTFTLPAPPSGTQWYRVTDTCDWNDGASTFVAPGSETLIGGAGTTYGQCGQSLLLLISK

SEQ ID NO: 8 - protein, 500 aa, Thermus thermophilus
MELPRAFGLLLHPTSLPGPYGVGVLGQEARDFLRFLKEAGGRYWQVLPLGPTGYGDSPYQSFSAFAGNPYLIDLRPLAERGYVRLEDPGFPQGRVDYGLLYAWKWPALKEAFRGFKEKASPEEREAFAAFREREAWWLEDYALFMALKGAHGGLPWNRWPLPLRKREEKALREAKSALAEEVAFHAFTQWLFFRQWGALKAEAEALGIRIIGDMPIFVAEDSAEVWAHPEWFHLDEEGRPTVVAGVPPDYFSETGQRWGNPLYRWDVLEREGFSFWIRRLEKALELFHLVRIDHFRGFEAYWEIPASCPTAVEGRWVKAPGEKLFQKIQEVFGEVPVLAEDLGVITPEVEALRDRFGLPGMKVLQFAFDDGMENPFLPHNYPAHGRVVVYTGTHDNDTTLGWYRTATPHEKAFMARYLADWGITFREEEEVPWALMHLGMKSVARLAVYPVQDVLALGSEARMNYPGRPSGNWAWRLLPGELSPEHGARLRAMAEATERL

SEQ ID NO: 9 - protein, 463 aa, Thermococcus gammatolerans
MRRYTRVLILLMALFLLAGLYYPSASAAKYSELEQGGVIMQAFYWDVPAGGIWWDTIRQKIPEWYDAGISAIWIPPASKGMGGAYSMGYDPYDYFDLGEFYQKGTVETRFGSKEELVNMISTAHRYGIKVIADIVINHRAGGDLEWNPYVGDYTWTDFSQVASGKYKAHYMDFHPNNYSTSDEGTFGGFPDIDHLVPFNKYWLWASDESYAAYLRSIGVDAWRFDYVKGYGAWVVKDWLSWWGGWAVGEYWDTDVNALLNWAYDSGAKVFDFPLYYKMDEAFDNKNIPALVYAIQNGGTVVSRDPFKAVTFVANHDTNIIWNKYPAYAFILTYEGQPVIFYRDYEEWLNKDKLNNLIWIHEHLAGGSTKILYYDDDELIFMREGYGDRPGLITYINLGSGWAERWVNVGSKFAGYTIHEYTGNLGGWVDRYVYYNGWVKLTAPPHDPANGYYGYSVWSYAGVG

SEQ ID NO: 10 - protein, 457 aa, Thermococcus thioreducens
MARKVTVALLVLLVVLSLSAVPAKAETLENGGVIMQAFYWDVPMGGIWWDTIAQKIPDWASAGISAIWIPPASKGMSGGYSMGYDPYDYFDLGEYYQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASNESYAAYLRSIGIDAWRFDYVKGYAPWVVKDWLNWWGGWAVGEYWDTNVDALLNWAYASGAKVFDFPLYYKMDEAFDNNNIPALVDALRYGQTVVSRDPFKAVTFVANHDTDIIWNKYPAYAFILTYEGQPMIFYRDYEEWLNKDRLKNLIWIHDHLAGGSTDIVYYDSDELIFVRNGYGSKPGLITYINLGSSKAGRWVYVPKFAGSCIHEYTGNLGGWVDKWVDSSGWVYLEAPAHDPANGQYGYSVWSYCGVG

SEQ ID NO: 11 - protein, 461 aa, Thermococcus eurythermalis
MKPAKLLVFVLVVSILAGLYAQPAGAAKYLELEEGGVIMQAFYWDVPSGGIWWDTIRQKIPEWYDAGISAIWIPPASKGMGGAYSMGYDPYDFFDLGEYDQKGTVETRFGSKQELVNMINTAHAYGIKVIADIVINHRAGGDLEWNPFVNDYTWTDFSKVASGKYTANYLDFHPNEVKCCDEGTFGGFPDIAHEKSWDQYWLWASNESYAAYLRSIGVDAWRFDYVKGYGAWVVKDWLDWWGGWAVGEYWDTNVDALLNWAYSSDAKVFDFPLYYKMDAAFDNKNIPALVEALKNGGTVVSRDPFKAVTFVANHDTDIIWNKYPAYAFILTYEGQPTIFYRDYEEWLNKDRLKNLIWIHDHLAGGSTDIVYYDNDELIFVRNGYGDKPGLITYINLGSSKAGRWVYVPKFAGACIHEYTGNLGGWVDKWVDSSGWVYLEAPAHDPANGYYGYSVWSYCGVG

SEQ ID NO: 12 - protein, 457 aa, Thermococcus hydrothermalis
MARKVLVALLVFLVVLSVSAVPAKAETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 13 - protein, 460 aa, Pyrococcus furiosus
MNIKKLTPLLTLLLFFIVLASPVSAAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 14 - DNA, 1641 bp, Artificial Sequence
  Gene encoding FLO1 protein
  CDS (1)..(1641)
aaggacaatagctcgacgattgaaggtagatacccatacgacgttccagactacgctctgcaggctagtggtggtggtggttctggtggtggtggttctggtggtggtggttctgctagcatcagaactccaaccagtgaaggtttggttacaaccaccactgaaccatggactggtacttttacttcgacttccactgaaatgtctactgtcactggaaccaatggcttgccaactgatgaaactgtcattgttgtcaaaactccaactactgccatctcatccagtttgtcatcatcatcttcaggacaaatcaccagctctatcacgtcttcgcgtccaattattaccccattctatcctagcaatggaacttctgtgatttcttcctcagtaatttcttcctcagtcacttcttctctattcacttcttctccagtcatttcttcctcagtcatttcttcttctacaacaacctccacttctatattttctgaatcatctaaatcatccgtcattccaaccagtagttccacctctggttcttctgagagcgaaacgagttcagctggttctgtctcttcttcctcttttatctcttctgaatcatcaaaatctcctacatattcttcttcatcattaccacttgttaccagtgcgacaacaagccaggaaactgcttcttcattaccacctgctaccactacaaaaacgagcgaacaaaccactttggttaccgtgacatcctgcgagtctcatgtgtgcactgaatccatctcccctgcgattgtttccacagctactgttactgttagcggcgtcacaacagagtataccacatggtgccctatttctactacagagacaacaaagcaaaccaaagggacaacagagcaaaccacagaaacaacaaaacaaaccacggtagttacaatttcttcttgtgaatctgacgtatgctctaagactgcttctccagccattgtatctacaagcactgctactattaacggcgttactacagaatacacaacatggtgtcctatttccaccacagaatcgaggcaacaaacaacgctagttactgttacttcctgcgaatctggtgtgtgttccgaaactgcttcacctgccattgtttcgacggccacggctactgtgaatgatgttgttacggtctatcctacatggaggccacagactgcgaatgaagagtctgtcagctctaaaatgaacagtgctaccggtgagacaacaaccaatactttagctgctgaaacgactaccaatactgtagctgctgagacgattaccaatactggagctgctgagacgaaaacagtagtcacctcttcgctttcaagatctaatcacgctgaaacacagacggcttccgcgaccgatgtgattggtcacagcagtagtgttgtttctgtatccgaaactggcaacaccaagagtctaacaagttccgggttgagtactatgtcgcaacagcctcgtagcacaccagcaagcagcatggtaggatatagtacagcttctttagaaatttcaacgtatgctggcagtgccaacagcttactggccggtagtggtttaagtgtcttcattgcgtccttattgctggcaattatttaa

SEQ ID NO: 15 - protein, 546 aa, Artificial Sequence
  Synthetic Construct
KDNSSTIEGRYPYDVPDYALQASGGGGSGGGGSGGGGSASIRTPTSEGLVTTTTEPWTGTFTSTSTEMSTVTGTNGLPTDETVIVVKTPTTAISSSLSSSSSGQITSSITSSRPIITPFYPSNGTSVISSSVISSSVTSSLFTSSPVISSSVISSSTTTSTSIFSESSKSSVIPTSSSTSGSSESETSSAGSVSSSSFISSESSKSPTYSSSSLPLVTSATTSQETASSLPPATTTKTSEQTTLVTVTSCESHVCTESISPAIVSTATVTVSGVTTEYTTWCPISTTETTKQTKGTTEQTTETTKQTTVVTISSCESDVCSKTASPAIVSTSTATINGVTTEYTTWCPISTTESRQQTTLVTVTSCESGVCSETASPAIVSTATATVNDVVTVYPTWRPQTANEESVSSKMNSATGETTTNTLAAETTTNTVAAETITNTGAAETKTVVTSSLSRSNHAETQTASATDVIGHSSSVVSVSETGNTKSLTSSGLSTMSQQPRSTPASSMVGYSTASLEISTYAGSANSLLAGSGLSVFIASLLLAII

SEQ ID NO: 16 - DNA, 807 bp, Artificial Sequence
  SED1 tether
  CDS (1)..(807)
aaggacaatagctcgacgattgaaggtagatacccatacgacgttccagactacgctctgcaggctagtggtggtggtggttctggtggtggtggttctggtggtggtggttctgctagcgctcttccaactaacggtacttctactgaagctccaactgatactactactgaagctccaaccaccggtcttccaaccaacggtaccacttcagctttcccaccaactacatctttgccaccaagcaacactaccaccactcctccttacaacccatctactgactacaccactgactacactgtagtcactgaatatactacttactgtccagaaccaaccactttcaccacaaacggtaagacttacaccgtcactgaaccaaccacattgactatcactgactgtccatgcaccattgaaaagccaacaaccacatcaaccaccgaatacactgtagtcactgagtacactacttactgtccagaaccaaccactttcaccacaaacggtaagacttacaccgtcactgaaccaaccactttgactatcactgactgtccatgtactattgaaaagagcgaagcccctgagtcttctgtcccagttaccgaatctaagggcactaccaccaaagaaacaggtgttactaccaaacaaaccacagccaacccaagtctaaccgtctccacagtcgtcccagtttcatcctctgcttcttctcattccgttgtcatcaacagtaacggtgctaacgtcgtcgttccaggtgctttaggtttggctggtgttgctatgttattctaa

SEQ ID NO: 17 - protein, 268 aa, Artificial Sequence
  Synthetic Construct
KDNSSTIEGRYPYDVPDYALQASGGGGSGGGGSGGGGSASALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLF

SEQ ID NO: 18 - DNA, 390 bp, Artificial Sequence
  SPI1 tether
  CDS (1)..(390)
ttggtatctaattctagttcctctgtaatcgtggtaccatcaagcgatgctactattgccggtaacgatacagccacgccagcaccagagccatcatccgccgctccaatattctacaactcgactgctactgcaacacagtacgaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 19 - protein, 129 aa, Artificial Sequence
  Synthetic Construct
LVSNSSSSVIVVPSSDATIAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 20 - DNA, 339 bp, Artificial Sequence
  CCW12 tether
  CDS (1)..(339)
gttaccactgctactgtcagccaagaatctaccactttggtcaccatcacttcttgtgaagaccacgtctgttctgaaactgtctccccagctttggtttccaccgctaccgtcaccgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 21 - protein, 112 aa, Artificial Sequence
  Synthetic Construct
VTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 22 - DNA, 450 bp, Artificial Sequence
  CWP2 tether
  CDS (1)..(450)
aaggacaatagctcgacgattgaaggtagatacccatacgacgttccagactacgctctgcaggctagtggtggtggtggttctggtggtggtggttctggtggtggtggttctgctagcggatccggtggcggtggatctggaggaggcggttcttggtctcacccacaatttgaaaagggtggagaaaacttgtactttcaaggcggtggtggaggttctggcggaggtggctccggctcagctatctctcaaatcaccgacggtcaaatccaagccactaccacagctaccactgaagctacaactaccgctgctccttcatctactgttgaaactgtttctccatcttccaccgaaaccatctctcaacaaaccgaaaacggtgctgctaaggctgctgttggtatgggtgctggtgctttggctgctgctgctatgttgttgtaa

SEQ ID NO: 23 - protein, 149 aa, Artificial Sequence
  Synthetic Construct
KDNSSTIEGRYPYDVPDYALQASGGGGSGGGGSGGGGSASGSGGGGSGGGGSWSHPQFEKGGENLYFQGGGGGSGGGGSGSAISQITDGQIQATTTATTEATTTAAPSSTVETVSPSSTETISQQTENGAAKAAVGMGAGALAAAAMLL

SEQ ID NO: 24 - DNA, 759 bp, Artificial Sequence
  TIR1 tether
  CDS (1)..(759)
aaggacaatagctcgacgattgaaggtagatacccatacgacgttccagactacgctctgcaggctagtggtggtggtggttctggtggtggtggttctggtggtggtggttctgctagcagcttagcttctgattcttcctctggattttccttaagcagtatgccagctggtgttttggatatcggtatggctttagcttccgccactgacgactcctacactactttgtactctgaggttgactttgctggtgttagcaagatgttgaccatggttccatggtactcctctagattggaaccagctttgaagtctttgaatggtgatgcttcttcttctgctgccccaagctcttctgctgctccaacttcttctgctgccccaagctcatctgctgccccaacttcttctgctgcctcaagctcttctgaagctaagtcttcttctgctgccccaagctcttctgaagctaagtcttcttctgctgccccaagctcttctgaagctaagtcttcttctgctgccccaagctcttctgaagctaagtcttcttctgctgctccaagctccactgaagctaagataacttctgctgctccaagctccactggtgccaagacctctgccatctctcaaattaccgatggtcaaatccaagctaccaaggctgtttctgagcaaactgaaaacggtgctgctaaggcctttgttggtatgggtgctggtgttgtcgcagctgccgctatgttgttataa

SEQ ID NO: 25 - protein, 252 aa, Artificial Sequence
  Synthetic Construct
KDNSSTIEGRYPYDVPDYALQASGGGGSGGGGSGGGGSASSLASDSSSGFSLSSMPAGVLDIGMALASATDDSYTTLYSEVDFAGVSKMLTMVPWYSSRLEPALKSLNGDASSSAAPSSSAAPTSSAAPSSSAAPTSSAASSSSEAKSSSAAPSSSEAKSSSAAPSSSEAKSSSAAPSSSEAKSSSAAPSSTEAKITSAAPSSTGAKTSAISQITDGQIQATKAVSEQTENGAAKAFVGMGAGVVAAAAMLL

SEQ ID NO: 26 - DNA, 1398 bp, Artificial Sequence
  PST1 tether
  CDS (1)..(1398)
aaggacaatagctcgacgattgaaggtagatacccatacgacgttccagactacgctctgcaggctagtggtggtggtggttctggtggtggtggttctggtggtggtggttctgctagcgctacttcctcttcttccagcataccctcttcctgtaccataagctcacatgccacggccacagctcagagtgacttagataaatatagccgctgtgatacgttagtcgggaacttaactattggtggtggtttgaagactggtgctttggctaatgttaaagaaatcaacgggtctctaactatatttaacgctacaaatctaacctcattcgctgctgattccttggagtccatcacagattctttgaacctacagagtttgacaatcttgacttctgcttcatttgggtctttacagagcgttgatagtataaaactgattactctacccgccatctccagttttacttcaaatatcaaatctgctaacaacatttatatttccgacacttcgttacaatctgtcgatggattctcagccttgaaaaaagttaacgtgttcaacgtcaataacaataagaaattaacctcgatcaaatctccagttgaaacagtcagcgattctttacaattttcgttcaacggtaaccagactaaaatcaccttcgatgacttggtttgggcaaacaatatcagtttgaccgatgtccactctgtttccttcgctaacttgcaaaagattaactcttcattgggtttcatcaacaactccatctcaagtttgaatttcactaagctaaacaccattggccaaaccttcagtatcgtttccaatgactacttgaagaacttgtcgttctctaatttgtcaaccataggtggtgctcttgtcgttgctaacaacactggtttacaaaaaattggtggtctcgacaacctaacaaccattggcggtactttggaagttgttggtaacttcacctccttgaacctagactctttgaagtctgtcaagggtggcgcagatgtcgaatcaaagtcaagcaatttctcctgtaatgctttgaaagctttgcaaaagaaagggggtatcaagggtgaatcttttgtctgcaaaaatggtgcatcatccacatctgttaaactatcgtccacttccaaatctcaatcaagccaaactactgccaaggtttccaagtcatcttctaaggccgaggaaaagaagttcacttctggcgatatcaaggctgctgcttctgcctctagtgtttctagttctggcgcttccagctctagctctaagagttccaaaggcaatgccgctatcatggcaccaattggccaaacaacccctttggtcggtcttttgacggcaatcatcatgtctataatgtaa

SEQ ID NO: 27 - protein, 465 aa, Artificial Sequence
  Synthetic Construct
KDNSSTIEGRYPYDVPDYALQASGGGGSGGGGSGGGGSASATSSSSSIPSSCTISSHATATAQSDLDKYSRCDTLVGNLTIGGGLKTGALANVKEINGSLTIFNATNLTSFAADSLESITDSLNLQSLTILTSASFGSLQSVDSIKLITLPAISSFTSNIKSANNIYISDTSLQSVDGFSALKKVNVFNVNNNKKLTSIKSPVETVSDSLQFSFNGNQTKITFDDLVWANNISLTDVHSVSFANLQKINSSLGFINNSISSLNFTKLNTIGQTFSIVSNDYLKNLSFSNLSTIGGALVVANNTGLQKIGGLDNLTTIGGTLEVVGNFTSLNLDSLKSVKGGADVESKSSNFSCNALKALQKKGGIKGESFVCKNGASSTSVKLSSTSKSQSSQTTAKVSKSSSKAEEKKFTSGDIKAAASASSVSSSGASSSSSKSSKGNAAIMAPIGQTTPLVGLLTAIIMSIM

SEQ ID NO: 28 - DNA, 330 bp, Artificial Sequence
  AGA1/2 tether
  CDS (1)..(330)
aaggacaatagctcgacgattgaaggtagatacccatacgacgttccagactacgctctgcaggctagtggtggtggtggttctggtggtggtggttctggtggtggtggttctgctagccaggaactgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcggttctcacccctcaacaactagcaaaggcagccccataaacacacagtatgttttttaa

SEQ ID NO: 29 - protein, 109 aa, Artificial Sequence
  Synthetic Construct
KDNSSTIEGRYPYDVPDYALQASGGGGSGGGGSGGGGSASQELTTICEQIPSPTLESTPYSLSTTTILANGKAMQGVFEYYKSVTFVSNCGSHPSTTSKGSPINTQYVF

SEQ ID NO: 30 - DNA, 381 bp, Artificial Sequence
  AGA1/2 tether
  CDS (1)..(381)
atgcagttacttcgctgtttttcaatattttctgttattgcttcagttttagcacaggagctgacaactatatgcgagcaaatcccctcaccaactttagaatcgacgccgtactctttgtcaacgactactattttggccaacgggaaggcaatgcaaggagtttttgaatattacaaatcagtaacgtttgtcagtaattgcgattctcacccctcaacaactagcaaagacagccccataaacacacagtatgtttttaaggacaatagctcgacgattgaaggtagatacccatacgacgttccagactacgctctgcaggctagtggtggtggtggttctggtggtggtggttctggtggtggtggttctgctagc

SEQ ID NO: 31 - protein, 127 aa, Artificial Sequence
  Synthetic Construct
MQLLRCFSIFSVIASVLAQELTTICEQIPSPTLESTPYSLSTTTILANGKAMQGVFEYYKSVTFVSNCDSHPSTTSKDSPINTQYVFKDNSSTIEGRYPYDVPDYALQASGGGGSGGGGSGGGGSAS

SEQ ID NO: 32 - protein, 15 aa, Artificial Sequence
  Artificial linker
GGGGSGGGGSGGGGS

SEQ ID NO: 33 - protein, 8 aa, Artificial Sequence
  Artificial linker
GGGGGGGG

SEQ ID NO: 34 - protein, 40 aa, Artificial Sequence
  Artificial linker
GGGGSGGGGSGGGGSGGGGSGGGGSGGGGSGGGGSGGGGS

SEQ ID NO: 35 - protein, 12 aa, Artificial Sequence
  Artificial Linker
GSAGSAAGSGEF

SEQ ID NO: 36 - protein, 12 aa, Artificial Sequence
  Artificial linker
EAAKEAAKEAAK

SEQ ID NO: 37 - protein, 20 aa, Artificial Sequence
  Artificial Linker
APAPAPAPAPAPAPAPAPAP

SEQ ID NO: 38 - protein, 46 aa, Artificial Sequence
  Artificial linker
AEAAAKEAAAKEAAAKEAAAKALEAEAAAKEAAAKEAAAKEAAAKA

SEQ ID NO: 39 - protein, 19 aa, Saccharomyces cerevisiae
MLLQAFLFLLAGFAAKISA

SEQ ID NO: 40 - protein, 18 aa, Saccharomyces cerevisiae
MQLLRCFSIFSVIASVLA

SEQ ID NO: 41 - DNA, 450 bp, Artificial Sequence
  Promoter of hxt7 gene
ccagaaaggcaacgcaaaattttttttccagggaataaactttttatgacccactacttctcgtaggaacaatttcgggcccctgcgtgttcttctgaggttcatcttttacatttgcttctgctggataattttcagaggcaacaaggaaaaattagatggcaaaaagtcgtctttcaaggaaaaatccccaccatctttcgagatcccctgtaacttattggcaactgaaagaatgaaaaggaggaaaatacaaaatatactagaactgaaaaaaaaaaagtataaatagagacgatatatgccaatacttcacaatgttcgaatctattcttcatttgcagctattgtaaaataataaaacatcaagaacaaacaagctcaacttgtcttttctaagaacaaagaataaacacaaaaacaaaaagtttttttaattttaatcaaaaa

SEQ ID NO: 42 - DNA, 750 bp, Artificial Sequence
  Promoter of ctt1 gene
ttaactgcaaagacccgtcacctatgaataagatgactctcttgttggggtcaatctcctcagcggcaaaggcagcacctaaagttgctcctgttgtaaaaccgatagacccccacaacacttgcgagataccgtaggcgtccttaggaaagatagtttgattgataccgaaggcagacgtgccggtctcggaaatgataacatcaccttcttgcaagaacttggacaattcgttccacaaccactcttgtttcaagggcgtgctagcagatacacctttgtttgcgggagttttggttggtacgggaacgctcttgtagcccttaataacatcgggaataaccttcagtaagttttgtagtgcaaatttcatttgtacgccggggaacgtagcgttcttcacctttacgtaatcggaatgaaactccactacatttttagtcttgtaggagtaggaaaacgaacctgtgttaaaatcagagagcaaagcaccgaccgaaaggatcaaatcagccgactcaacggcctgtttcacgtctggtttggacagcgttcccacataaacaccgccatatctgggatgctgttcatctattgaccctttacctaaaggtgtcacaaaagctgggaattgcgtcaaatcaattaacttctgggtttcctttttaacgttgtgcctagaagcacaggcatccgatagtatgacagggtttttcgaattctggatcaattctagtacggtatcaataa

SEQ ID NO: 43 - DNA, 750 bp, Artificial Sequence
  Promoter of glo1 gene
tattaatccaaaaagtgaatctttgacacccctttctcctatcttgcttagtaccgagtgatcttttgcataatcaaaacaagtgaagaaggtcgagttttgaaccttctctaatataagtcgacggtctttcgtgatggcgtcaatattacttagtcttttgtacttcacatataccattaatggcgtcaatcccttataattttcctcattaatatctagcttagtcagttgtaaaaggattggaatgtttgttttcaaaacatgcaacaagctattgcccttattatcggtgtggtctagatagtcaaacaaactgttgtgctttctataccaagtatttgcgatatcaaatgcggtcttcaccatttcttcataatttggttgatcataacttctaaagatggaaaataaaggcgtttgtccactggagttttttcgtttccaatcaatataatttccgatgcttttaagtatatccatttcatgggttagataatgagccacggtacgtgcatatttatctgttttattgatataagaaaccagctcctcttcagtacaattgaacagcatgatctttatgagaactttcgcggcttctaaatttcctgcttttatcgattctataagcagcgtagaaccatcaatcgtttcgtcctctaaaaggtcttctactggaaagtcgttttcatattcagaaagaatatctaataagatgtagtttttatgatttgttatacatatgga

SEQ ID NO: 44 - DNA, 750 bp, Artificial Sequence
  Promoter of ygp1 gene
tctcataattggtcagaagaaaggaaatctaggcaattgagttctttgggtaaaaaagagtcacaatttttaaggctgcgtagaacgagattatcgttagaagatttccacactgtgaaagtcattggaaaaggggcatttggtgaagtcagactggttcagaagaaagacaccggaaaaatatatgcaatgaaaactttattaaaatccgaaatgtacaaaaaggaccaattagcacacgtcaaggctgaaagggatgttctggctggaagtgattctccatgggtggtttcgctatattactcattccaagatgctcaatacttatatttaatcatggaatttttgcccggtggtgatttgatgaccatgctaatcaggtggcaactatttacagaggatgttactagattttacatggctgagtgtattttggccatcgaaaccattcataaattaggattcattcacagagatattaaaccagataatattttgatcgatattagaggtcatataaaattatctgatttcgggttgtctacggggttccataaaactcacgactcaaactactataaaaagcttttgcaacaagatgaggcaactaatggtatttccaagccaggtacttacaatgcaaatacaaccgatactgcaaataaaaggcaaacaatggttgtggattctattagtctaacaatgtcaaacaggcaacaaattcagacatggagaaaatcacgtcgttta

SEQ ID NO: 45 - DNA, 750 bp, Artificial Sequence
  Promoter of gsy2 gene
tattatagctcttgataacaatgaaatttcaacaattattcactcaaccctcaagtgtatatcgccaagatcctaatcacccctctctggtggccttccctcattgtttttacacagaacaagaatccataaaatcaagatattgggaacaattcattgtcttaggaacaagtttttataatttagtttcattagcctattgtaacttcttgtttttttttttattttggctggcaaaacaagaagtataaagaagttaccactgctataacgtatatcttaaatcaagtgggcaattcttcgaataaacttagaggcgctgaagaaagaaccgctcaattagttcgatcatacaaaatacagatattgcgctgtgtattatcattacagaaagtgtgatataaacaatgctgtgttgtattcggtcttttttttccgcctgcagattgtcgtttttgtttttttttctcgtgtagctgcagttatcatcgttttgcatgtctgttctgtcggtgaagccgatctcatggcacttcgctttgataccgtaaagaatcttcttcattaatacggcactgcaaggcgatatctatctctggggttaggactgataaataggtacttatcagttatcactagagacgaactgtttacgttttgaagctcgacagcaggggttcttaacgggcgtcggcctagacctcaccttgcgagaaatttttagttcatatccaggggatggtgaatggt

SEQ ID NO: 46 - DNA, 750 bp, Artificial Sequence
  Promoter of mol1/thi4 gene
tcgttgaaagttctagatttgggtacggaagcagccatgtaaatggtcttcctttgtaataatatcgtcactggagtttttaaattagagtacggattcaagttaacgaaaaatttctgttggtatctgatagcaatttgtcgacaaggtggagagtttttgaagtttttttttgtaacagttatgttacactatttaccagagaaacaccaagacaggagccctcagaagctttcgtgttgtcagaacataaatatcttctatatgttttattcttttatattaatcaatattttatgttaaatcatattattattcgttaatgcttaacttgccctatttcaaaatcggaagggtagtatggtgacaagcggtgtttttggtgtatgtttactgctaagagtataccgtcctgtttacttttttaccttttgttccgttattgatttttcgcagcaaggttgaagcttccatatttgaaaatgaaattaagaagtagagatgaaaaacagcgacgaaattactttttatagcttctttcccttctgactccgttttataaacaggggatctttaagggcttacaaaataaatataaacactaacacatatatattattcgacctacagtttttgcgaccaatgaagtttacctattttgagatggtgcttacgtagtttgcttaaggtcaaggcgggattaagcttgcgctttctctaaataatatcatatgttacagacagggataa

SEQ ID NO: 47 - DNA, 750 bp, Artificial Sequence
  Promoter of sti1 gene
taccacaaagggtaaaacgtcaataaaagcaagcaatgttattcgtgccagagaactatatctggcgtacgtcttatatataacacaagcaagggcaaatacgtggttagtggtgtaaaaactgaagggattaatttacatacaactagatccatctttctcaaaaatgacttcagtatcgccctcgccacctgccagtcgatcgggctcaatgtgctccgacttaccgtcctctttgcagactgagaaactggcacatattataggtcttgatgccgacgatgaagttctccggcgcgtaaccaagcagttgagcagatctaggagaattgcttgtctgactggggcaggcatttcgtgcaacgcgggcattcctgactttcgctcttctgatgggctctacgacctagtgaaaaaggattgttcacagtattggtctatcaagtccggcagggaaatgtttgatatttcgctatttagagatgacttcaaaatatccatttttgctaaatttatggagaggctctattcaaatgttcaattggcaaagccgactaagacgcacaagttcattgcgcatctaaaagataggaacaaactgctgcgctgttacacgcaaaacatcgatgggctcgaagaaagcataggacttactttatcaaataggaaattaccgcttacctcatttagttcacattggaaaaatctggatgtcgttcagttgcacggcgacctgaaaactctttcgtg

SEQ ID NO: 48 - protein, 4 aa, Artificial Sequence
  Artificial sequence EA2K motif
EAAK

SEQ ID NO: 49 - protein, 5 aa, Artificial Sequence
  Artificial sequence EA3K motif
EAAAK

SEQ ID NO: 50 - protein, 22 aa, Artificial Sequence
  HA Tag
KDNSSTIEGRYPYDVPDYALQA

SEQ ID NO: 51 - DNA, 750 bp, Artificial Sequence
  Promoter of cup1 gene
taaatatatgctgagatttagcaagaaaaaacaaaattttgcgagaaactctgataacaataatgttacagattatagtcagtcggcgaagaacaaaaatgttctcttgaaattccccgttagtgaactgaacagaatctatttaaaatttaaggagatttcagattttttaatggaaagagaagttgtccaaaggagtataattattgacaaggatttggaatctgataatctgggtattactacggcaaacttcaacgatttctatgatgcattttataattagtaagccgatcccattaccgacatttgggcgctatacgtgcatatgttcatgtatgtatctgtatttaaaacacttttgtattatttttcctcatatatgtgtataggtttatacggatgatttaattattacttcaccaccctttatttcaggctgatatcttagccttgttactagttagaaaaagacatttttgctgtcagtcactgtcaagagattcttttgctggcatttcttctagaagcaaaaagagcgatgcgtcttttccgctgaaccgttccagcaaaaaagactaccaacgcaatatggattgtcagaatcatataaaagagaagcaaataactccttgtcttgtatcaattgcattataatatcttcttgttagtgcaatatcatatagaagtcatcgaaatagatattaagaaaaacaaactgtacaatcaatcaatcaatcatcacataaa

SEQ ID NO: 52 - DNA, 750 bp, Artificial Sequence
  Promoter of trx2 gene
ataaccacaatgatcgcagaccgtcgacatgataatgacttctttaaagtgtgggatatttactggcttcatatgagtttcacactcttgggtacacgatggacaagaagctctgaatgtttgcacctcgttgttaaagttttcgatgtcggtggcgtctgacaaaaactgtgggtttgctgagccaactttgacagattcagaaggatttctttcacggttggccaattgttttaattgttcttggcgacgttgctccaactggtctctagtaataatgccaacttgaacgttttgttcgtcggttctcacgtactgggtatgagaccatttgtgttgaggttcaccgggtttgtattcgatccaggaatttcccgcaggatcatccaaaataaatgtaatcggaatagtgttgggttcacaattgatgtaggatttaactttctgtacgaagtcatcgatcttcttgtaaagagcttcgtctatagattttctcatttcctggtcttgcgacagatcgtcgatcatctcggataacaaaccttcaactgtcgtcaattgacctctcttagcaggaatctcaatgtctagctcgacaaacttacaagtggcagtttctgacttaataacttgcctgttaaaatcttcacggcactccactttcaaaacgtaacgagagcccttctcttgaatttgagaagcgggttggatctcacagttcttaaacccacagtgaggacagtcgaatga

SEQ ID NO: 53 - DNA, 750 bp, Artificial Sequence
  Promoter of gpd1 gene
aaggaaaatatatactctttcccaggcaaggtgacagcggtccccgtctcctccacaaaggcctctcctggggtttgagcaagtctaagtttacgtagcataaaaattctcggattgcgtcaaataataaaaaaagtaactccacttctacttctacatcggaaaaacattccattcacatatcgtctttggcctatcttgttttgtccttggtagatcaggtcagtacaaacgcaacacgaaagaacaaaaaaagaagaaaaacagaaggccaagacagggtcaatgagactgttgtcctcctactgtccctatgtctctggccgatcacgcgccattgtccctcagaaacaaatcaaacacccacaccccgggcacccaaagtccccacccacaccaccaatacgtaaacggggcgccccctgcaggccctcctgcgcgcggcctcccgccttgcttctctcccctcccttttctttttccagttttccctattttgtccctttttccgcacaacaagtatcagaatgggttcatcaaatctatccaacctaattcgcacgtagactggcttggtattggcagtttcgcagttatatatatactaccatgagtgaaactgttacgttaccttaaattctttctccctttaattttcttttatcttactctcctacataagacatcaagaaacaattgtatattgtacacccccccccctccacaaacacaaatattgataatataaag

SEQ ID NO: 54 - DNA, 750 bp, Artificial Sequence; identical to SEQ ID NO: 53
  Promoter of hsp12 gene

SEQ ID NO: 55 - DNA, 510 bp, Artificial Sequence
  SPI tethering moiety with HA tag and linker
  CDS (1)..(510)
aaggacaatagctcgacgattgaaggtagatacccatacgacgttccagactacgctctgcaggctagtggtggtggtggttctggtggtggtggttctggtggtggtggttctgctagcttggtatctaattctagttcctctgtaatcgtggtaccatcaagcgatgctactattgccggtaacgatacagccacgccagcaccagagccatcatccgccgctccaatattctacaactcgactgctactgcaacacagtacgaagttgtcagtgaattcactacttactgcccagaaccaacgactttcgtaacgaatggcgctacattcactgttactgccccaactacgttaacaattaccaactgtccttgcactatcgagaagcctacttcagaaacatcggtttcttctacacatgatgtggagacaaattctaatgctgctaacgcaagagcaatcccaggagccctaggtttggctggtgcagttatgatgcttttatga

SEQ ID NO: 56 - protein, 169 aa, Artificial Sequence
  Synthetic Construct
KDNSSTIEGRYPYDVPDYALQASGGGGSGGGGSGGGGSASLVSNSSSSVIVVPSSDATIAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 57 - DNA, 468 bp, Artificial Sequence
  CCW12 tethering moiety with HA Tag and Linker
  CDS (1)..(468)
aaggacaatagctcgacgattgaaggtagatacccatacgacgttccagactacgctctgcaggctagtggtggtggtggttctggtggtggtggttctggtggtggtggttctgctagcgctgctaacgttaccactgctactgtcagccaagaatctaccactttggtcaccatcacttcttgtgaagaccacgtctgttctgaaactgtctccccagctttggtttccaccgctaccgtcaccgtcgatgacgttatcactcaatacaccacctggtgcccattgaccactgaagccccaaagaacggtacttctactgctgctccagttacctctactgaagctccaaagaacaccacctctgctgctccaactcactctgtcacctcttacactggtgctgctgctaaggctttgccagctgctggtgctttgttggctggtgccgctgctttgttgttgtaa

SEQ ID NO: 58 - protein, 155 aa, Artificial Sequence
  Synthetic Construct
KDNSSTIEGRYPYDVPDYALQASGGGGSGGGGSGGGGSASAANVTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 59 - protein, 20 aa, Aspergillus terreus
MKWTFSLLLLLSVFGQATHA

SEQ ID NO: 60 - protein, 435 aa, Thermococcus gammatolerans
KYSELEQGGVIMQAFYWDVPAGGIWWDTIRQKIPEWYDAGISAIWIPPASKGMGGAYSMGYDPYDYFDLGEFYQKGTVETRFGSKEELVNMISTAHRYGIKVIADIVINHRAGGDLEWNPYVGDYTWTDFSQVASGKYKAHYMDFHPNNYSTSDEGTFGGFPDIDHLVPFNKYWLWASDESYAAYLRSIGVDAWRFDYVKGYGAWVVKDWLSWWGGWAVGEYWDTDVNALLNWAYDSGAKVFDFPLYYKMDEAFDNKNIPALVYAIQNGGTVVSRDPFKAVTFVANHDTNIIWNKYPAYAFILTYEGQPVIFYRDYEEWLNKDKLNNLIWIHEHLAGGSTKILYYDDDELIFMREGYGDRPGLITYINLGSGWAERWVNVGSKFAGYTIHEYTGNLGGWVDRYVYYNGWVKLTAPPHDPANGYYGYSVWSYAGVG

SEQ ID NO: 61 - protein, 432 aa, Thermococcus thioreducens
ETLENGGVIMQAFYWDVPMGGIWWDTIAQKIPDWASAGISAIWIPPASKGMSGGYSMGYDPYDYFDLGEYYQKGTVETRFGSKQELVNMINTAHAYGMKVIADIVINHRAGGDLEWNPFVNDYTWTDFSKVASGKYTANYLDFHPNELHAGDSGTFGGYPDICHDKSWDQYWLWASNESYAAYLRSIGIDAWRFDYVKGYAPWVVKDWLNWWGGWAVGEYWDTNVDALLNWAYASGAKVFDFPLYYKMDEAFDNNNIPALVDALRYGQTVVSRDPFKAVTFVANHDTDIIWNKYPAYAFILTYEGQPMIFYRDYEEWLNKDRLKNLIWIHDHLAGGSTDIVYYDSDELIFVRNGYGSKPGLITYINLGSSKAGRWVYVPKFAGSCIHEYTGNLGGWVDKWVDSSGWVYLEAPAHDPANGQYGYSVWSYCGVG

SEQ ID NO: 62 - protein, 440 aa, Thermococcus eurythermalis
QPAGAAKYLELEEGGVIMQAFYWDVPSGGIWWDTIRQKIPEWYDAGISAIWIPPASKGMGGAYSMGYDPYDFFDLGEYDQKGTVETRFGSKQELVNMINTAHAYGIKVIADIVINHRAGGDLEWNPFVNDYTWTDFSKVASGKYTANYLDFHPNEVKCCDEGTFGGFPDIAHEKSWDQYWLWASNESYAAYLRSIGVDAWRFDYVKGYGAWVVKDWLDWWGGWAVGEYWDTNVDALLNWAYSSDAKVFDFPLYYKMDAAFDNKNIPALVEALKNGGTVVSRDPFKAVTFVANHDTDIIWNKYPAYAFILTYEGQPTIFYRDYEEWLNKDRLKNLIWIHDHLAGGSTDIVYYDNDELIFVRNGYGDKPGLITYINLGSSKAGRWVYVPKFAGACIHEYTGNLGGWVDKWVDSSGWVYLEAPAHDPANGYYGYSVWSYCGVG

SEQ ID NO: 63 - protein, 432 aa, Thermococcus hydrothermalis
ETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVG

SEQ ID NO: 64 - protein, 434 aa, Pyrococcus furiosus
KYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 65 - protein, 622 aa, Pyrococcus furiosus
MLLQAFLFLLAGFAAKISAAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVGKDNSSTIEGRYPYDVPDYALQASGGGGSGGGGSGGGGSALVSNSSSSVIVVPSSDATIAGNDTATPAPEPSSAAPIFYNSTATATQYEVVSEFTTYCPEPTTFVTNGATFTVTAPTTLTITNCPCTIEKPTSETSVSSTHDVETNSNAANARAIPGALGLAGAVMMLL

SEQ ID NO: 66 - protein, 614 aa, Thermococcus hydrothermalis
MRFPSIFTAVLFAASSALAAPETLENGGVIMQAFYWDVPGGGIWWDTIAQKIPDWASAGISAIWIPPASKGMSGGYSMGYDPYDFFDLGEYYQKGSVETRFGSKEELVNMINTAHAHNMKVIADIVINHRAGGDLEWNPFTNSYTWTDFSKVASGKYTANYLDFHPNELHAGDSGTFGGYPDICHDKSWDQHWLWASNESYAAYLRSIGIDAWRFDYVKGYAPWVVKNWLNRWGGWAVGEYWDTNVDALLSWAYDSGAKVFDFPLYYKMDEAFDNNNIPALVDALKNGGTVVSRDPFKAVTFVANHDTNIIWNKYPAYAFILTYEGQPAIFYRDYEEWLNKDRLRNLIWIHDHLAGGSTDIIYYDSDELIFVRNGYGDKPGLITYINLGSSKAGRWVYVPKFAGSCIHEYTGNLGGWIDKWVDSSGRVYLEAPAHDPANGQYGYSVWSYCGVGAEAAAKEAAAKEAAAKEAAAKALEAEAAAKEAAAKEAAAKEAAAKAAANVTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 67 - protein, 618 aa, Rasamsonia emersonii
MASLVAGALCILGLTPAAFARAPVAARATGSLDSFLATETPIALQGVLNNIGPNGADVAGASAGIVVASPSRSDPNYFYSWTRDAALTAKYLVDAFIAGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVPAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 68 - protein, 494 aa, Saccharomycopsis fibuligera
MQISKAALLASLAALVYAQPVTLFKRETNADKWRSQSIYQIVTDRFARTDGDTSASCNTEDRLYCGGSFQGIIKKLDYIKDMGFTAIWISPVVENIPDNTAYGYAYHGYWMKNIYKINENFGTADDLKSLAQELHDRDMLLMVDIVTNHYGSDGSGDSIDYSEYTPFNDQKYFHNYCLISNYDDQAQVQSCWEGDSSVALPDLRTEDSDVASVFNSWVKDFVGNYSIDGLRIDSAKHVDQGFFPDFVSASGVYSVGEVFQGDPAYTCPYQNYIPGVSNYPLYYPTTRFFKTTDSSSSELTQMISSVASSCSDPTLLTNFVENHDNERFASMTSDQSLISNAIAFVLLGDGIPVIYYGQEQGLSGKSDPNNREALWLSGYNKESDYYKLIAKANAARNAAVYQDSSYATSQLSVIFSNDHVIATKRGSVVSVFNNLGSSGSSDVTISNTGYSSGEDLVEVLTCSTVSGSSDLQVSIQGGQPQIFVPAKYASDICS

SEQ ID NO: 69 - protein, 659 aa, Bacillus amyloliquefaciens
MFEKRFKTSLLPLFAGFLLLFHLVLSGPAAANAETANKSNKVTASSVKNGTILHAWNWSFNTLTQNMKDIRDAGYAAIQTSPINQVKEGNQGDKSMRNWYWLYQPTSYQIGNRYLGTEQEFKDMCAAAEKYGVKVIVDAVINHTTSDYGAISDEIKRIPNWTHGNTQIKNWSDRWDVTQNSLLGLYDWNTQNTEVQVYLKRFLERALNDGADGFRYDAAKHIELPDDGNYGSQFWPNITNTSAEFQYGEILQDSASRDTAYANYMNVTASNYGHSIRSALKNRNLSVSNISHYASDVSADKLVTWVESHDTYANDDEESTWMSDDDIRLGWAVIGSRSGSTPLFFSRPEGGGNGVRFPGKSQIGDRGSALFKDQAITAVNTFHNVMAGQPEELSNPNGNNQVFMNQRGSKGVVLANAGSSSVTINTSAKLPDGRYDNRAGAGSFQVANGKLTGTINARSAAVLYPDDIGNAPHVFLENYQTGAVHSFNDQLTVTLRANAKTTKAVYQINNGQQTAFKDGDRLTIGKGDPIGTTYNIKLTGTNGEGAARTQEYTFVKKDPSQTNIIGYQNPDHWGQVNAYIYKHDGGRAIELTGSWPGKAMTKNANGMYTLTLPENTDTANAKVIFNNGSAQVPGQNQPGFDYVQNGLYNNSGLNGYLPH

SEQ ID NO: 70 - protein, 1072 aa, Aspergillus fumigatus
MLSQNLATWVSLLACLPAAIGLPNNNDRVARSLKRHGGHGHKQVDTNSSHVYKTRFPGVTWDDDHWLLSTTTLDQGHYQSRGSIANGYLGINVASVGPFFELDVPVSGDVINGWPLYSRRQTFATIAGFFDYQPTTNGSNFPWLNQYGGESVISGIPHWSGLILDLGDGNYLDATVDNKTITDFRSTYDFKSGVLSWSYTWTPKCNKGSFNITYRLFAHKLHVNQAVVDMEITPSQGSEATVVNVIDGFSAVRTDFVESGQDNGALFSAVRPWGISNVTAYVYTNLTASAGVDLTSRALVNDKPYVHSNESSIAQAVDVKFRANETVRITKFVGAASSDAFPNPQQTAKQAVSAAMGAGYMGSLQSHVEEWASILLDGSVDSFVDPATGKLPDDDHILNSQIIAVANTYYLLQNTVGKNAIKAVSGAPVNVDSISVGGLTSDSYAGLVFWDADVWMQPGLVASHPEAAQRVTNYRTKLYPQALENINTAFTSSKNRTTFSPSAAIYPWTSGRFGNCTGTGPCWDYQYHLNGDIGLSLMYQWIASGDTKTFREQHFPIYDSVATMYSNIVQRNGSSWTLTNMTDPDEYANHIDAGGFTMPLISETLSYANSFRKQFGLEQNETWTEISENVLLIREDGVTLEYTTMNGTAVVKQADIVLVTYPLVYDNNYTAQHALNDLDYYANQQSPDGPAMTWAIFAITANDVSPSGCSAYTYHQDSYDPYMRAPFYQLSEQMIDDAGINGGTHPAYPFLTGHGGANQVVLMGYLGLRLLPDDAIHIDPNLPPQVSNLKYRTFYWRGWPISSSSNRTHTTISRAANLAPLDTADSRFANASIPVLVGDPSNSTAYRLPVTAPLVVPNRQIGFNNTIPGNMVQCRPVYSPNDYAPGQFPIAAVDGATSTKWRPSTANMSSLTVALADVEINSKVSGFHFNWWQAPPVNATVIFHDEMLEDPVAAMSSSHGNSRYRVVTTLTNIEQSQPYDAQSTDNNEVVLNTGNTTDVSLSQTVHTSRYATLLISGNQAGGEEGATVAEWAILGESKGSSSGHGNNKRRLDVRAAAALSALNDRRYRQFNA

SEQ ID NO: 71 - protein, 744 aa, Neurospora crassa
MTSQPSSGKGRGRNLSIDEYNVYDDAKTYYTTEDRHHNHRAGARTRTYSQNSLFKQFERLGLQKEPYRRGSHDESTIPQSRRFLIQVEPTLQSLQSQEDTDGNMQITIEDNGPKVLSLRTAASNGYNRFDIRGTYMLSNLLQELYLAKEYGRKQIILDEARLNENPVNRLSRLIKDHFWEGLTRRIDASSIEIAARDPKDWTDDPRPRIYIPRGAPEQHEYYTKVALDRPELRLDVQYLPEKITPEIVRDMNAKPGLLAVDMEEVVDPKTGEKTLRGRPFVVPGGRFNELYGWDSYMESLGLLVNDRVDLAKSMVQNFCFCIKHYGKILNATRSYYLCRSQPPFLTDMTLRVYDKIKHEPGALEFLRQSLLAAIKEYYSVWTAEPRLDPVTGLSRYRPEGLGVPPETEAGHFIHILEPYAKKHNMSFDEFVYAYNHGEIKEPTLDDYFMHDRAVRESGHDTTYRFEGICADLATIDLNSLLFKYETDIARTIRNVFHDKFEVPDDWLATNNPAASKLETSAMWDRRAKRRKLAIDKYLWNEEAGMYFDYNTATRKQCNYESATTFWALWAGVSNPKQAAAMVTKALPKLEAFGGLLSGTKESRGEIGLERPNRQWDYPYGWAPQQILAWTGLYRYGFNEEAERLAYKWLFMITKAFVDFNGVVVEKYDVTRPIDPHRVDAEYGNQGLDFKGVAKEGFGWVNASYVYGLQIVNAHMRRALGTLTPYETFMKAVEENRNKALSELV

SEQ ID NO: 72 - protein, 327 aa, Aspergillus niger [sequence omitted]

SEQ ID NO: 73 - protein, 433 aa, Citrobacter braakii
MSTFIIRLLIFSLLCGSFSIHAEEQNGMKLERVVIVSRHGVRAPTKFTPIMKDVTPDQWPQWDVPLGWLTPRGGELVSELGQYQRLWFTSKGLLNNQTCPSPGQVAVIADTDQRTRKTGEAFLAGLAPKCQIQVHYQKDEEKNDPLFNPVKMGKCSFNTLKVKNAILERAGGNIELYTQRYQSSFRTLENVLNFSQSETCKTTEKSTKCTLPEALPSEFKVTPDNVSLPGAWSLSSTLTEIFLLQEAQGMPQVAWGRITGEKEWRDLLSLHNAQFDLLQRTPEVARSRATPLLDMIDTALLTNGTTENRYGIKLPVSLLFIAGHDTNLANLSGALDLKWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 74 - protein, 390 aa, Saccharomycopsis fibuligera
MLFSKSLLLSVLASLSFAAPVEKREKTLTLDFDVKRISSKAKNVTVASSPGFRRNLRAASDAGVTISLENEYSFYLTTIEIGTPGQKLQVDVDTGSSDLWVPGQGTSSLYGTYDHTKSTSYKKDRSGFSISYGDGSSARGDWAQETVSIGGASITGLEFGDATSQDVGQGLLGIGLKGNEASAQSSNSFTYDNLPLKLKDQGLIDKAAYSLYLNSEDATSGSILFGGSDSSKYSGSLATLDLVNIDDEGDSTSGAVAFFVELEGIEAGSSSITKTTYPALLDSGTTLIYAPSSIASSIGREYGTYSYSYGGYVTSCDATGPDFKFSFNGKTITVPFSNLLFQNSEGDSECLVGVLSSGSNYYILGDAFLRSAYVYYDIDNSQVGIAQAKY

SEQ ID NO: 75 - protein, 395 aa, Aspergillus fumigatus
MVVFSKVTAVVVGLSTIVSAVPVVQPRKGFTINQVARPVTNKKTVNLPAVYANALTKYGGTVPDSVKAAASSGSAVTTPEQYDSEYLTPVKVGGTTLNLDFDTGSADLWVFSSELSASQSSGHAIYKPSANAQKLNGYTWKIQYGDGSSASGDVYKDTVTVGGVTAQSQAVEAASHISSQFVQDKDNDGLLGLAFSSINTVSPRPQTTFFDTVKSQLDSPLFAVTLKYHAPGTYDFGYIDNSKFQGELTYTDVDSSQGFWMFTADGYGVGNGAPNSNSISGIADTGTTLLLLDDSVVADYYRQVSGAKNSNQYGGYVFPCSTKLPSFTTVIGGYNAVVPGEYINYAPVTDGSSTCYGGIQSNSGLGFSIFGDIFLKSQYVVFDSQGPRLGFAPQA

SEQ ID NO: 76 - protein, 21 aa, Saccharomyces cerevisiae
MRFPSIFTAVLFAASSALAAP

SEQ ID NO: 77 - protein, 17 aa, Artificial Sequence
  Linker
SGGGGSGGGGSGGGGSA

SEQ ID NO: 78 - protein, 115 aa, Artificial Sequence
  Synthetic construct
AANVTTATVSQESTTLVTITSCEDHVCSETVSPALVSTATVTVDDVITQYTTWCPLTTEAPKNGTSTAAPVTSTEAPKNTTSAAPTHSVTSYTGAAAKALPAAGALLAGAAALLL

SEQ ID NO: 79 - protein, 16 aa, Artificial Sequence
  Amino acid linker
  VARIANT (3)...(16): Any one or all of amino acids 3-16 can either be present or absent.
GSGSGSGSGSGSGSGS

SEQ ID NO: 80 - protein, 24 aa, Artificial Sequence
  Amino acid linker
  VARIANT (4)...(24): Any one or all of amino acids 4-24 can either be present or absent.
GGSGGSGGSGGSGGSGGSGGSGGS

SEQ ID NO: 81 - protein, 32 aa, Artificial Sequence
  Amino acid linker
  VARIANT (5)...(32): any one or all of amino acids 5-32 can either be present or absent.
GGGSGGGSGGGSGGGSGGGSGGGSGGGSGGGS

SEQ ID NO: 82 - protein, 40 aa, Artificial Sequence; identical to SEQ ID NO: 34
  Amino acid linker
  VARIANT (6)...(40): any one or all of amino acids 6-40  can either be present or absent.

SEQ ID NO: 83 - protein, 32 aa, Artificial Sequence
  Amino acid linker
  VARIANT (5)...(32): any one or all of amino acids 5-32 can either be present or absent.
GGSGGGSGGGSGGGSGGGSGGGSGGGSGGGSG

SEQ ID NO: 84 - protein, 32 aa, Artificial Sequence
  Amino acid linker
  VARIANT (5)...(32): any one or all of amino acids any one or all of amino acids 5-32 can either be  present or absent.
GSATGSATGSATGSATGSATGSATGSATGSAT

(19) United States
(12) Patent Application Publication
(10) Pub. No.: US 2023/0091532 A1
(43) Pub. Date: Mar. 23, 2023

(54) INACTIVATED YEAST AND YEAST PRODUCT FOR IMPROVING FERMENTATION YIELD

(71) Applicant: Lallemand Hungary Liquidity Management LLC, Budapest (HU)

(72) Inventors: Aaron Argyros, Lebanon, NH (US); Charles F. Rice, Plainfield, NH (US)

(21) Appl. No.: 16/980,250

(22) PCT Filed: Mar. 13, 2019
(86) PCT No.: PCT/IB2019/052052

§ 371 Date: Sep. 11, 2020

Related U.S. Application Data
(60) Provisional application No. 62/642,517, filed on Mar. 13, 2018.

(51) Int. Cl. C12P 19/14; C12N 1/18; C12N 9/26; C12P 7/06

(52) U.S. Cl. CPC C12P 19/14; C12N 1/18; C12N 9/2414; C12P 7/06

(57) ABSTRACT

The present disclosure concerns using an inactivated yeast product made from a yeast host cell to increase the yield of a fermentation product from a fermenting yeast host cell. The inactivated yeast extract can be formulated as a liquefaction or fermentation additive and can be used to improve the yield of a fermented product such as ethanol.

### TECHNOLOGICAL FIELD

The present disclosure relates to yeast products that can be used for improving yields of a fermentation product.

### BACKGROUND

Saccharomyces cerevisiae is an important biocatalyst used in the commercial production of fuel ethanol. This organism is proficient in converting glucose to ethanol via fermentation, often to concentrations greater than 20% w/v. However, S. cerevisiae is unable to hydrolyze polysaccharides and therefore requires the exogenous addition of expensive enzymes to convert complex sugars to glucose. For example, in the US, the primary source of fuel ethanol is corn starch, which, regardless of the mashing process, requires the exogenous addition of both alpha-amylase and glucoamylase. The cost of the purified enzymes range from $0.02-0.04 per gallon, which at 14 billion gallons of ethanol produced each year, represents a substantial cost savings opportunity for producers if they could reduce their enzyme dose.

In a broad sense, there are two major fermentation processes in the corn ethanol industry: liquefied corn mash and raw corn flour. In the mash process, corn is both thermally and enzymatically liquefied using alpha-amylases prior to fermentation in order to break down long chain starch polymers into smaller dextrins. The mash is then cooled and inoculated with S. cerevisiae along with the exogenous addition of purified glucoamylase, an exo-acting enzyme which will further break down the dextrin into utilizable glucose molecules. In the raw flour process, the corn is only milled, not heated, creating a raw flour-like substrate which relies heavily on the addition of exogenous enzymes to complete the saccharification process. In either process, the addition of a robust, ethanol tolerant yeast strain is required to ferment the hydrolyzed starch into the desired final product, ethanol.

Yeast nutrients are commonly added during the fermentation process to ensure efficient fermentations. Yeast need exogenous nutrients for healthy growth and viability. Whereas the corn mash itself can provide some nutrients in the form of carbohydrates, fatty acids, and nitrogen, it does not provide sufficient nutrients for the necessary growth and metabolism in a typical fermentation. Proper nutrition also improves the cell's robustness and increases the likelihood that the cell will survive the harsh and variable fermentation conditions of high ethanol, fluctuating temperatures, and potential organic acids from contamination events. There are many nutrient products available on the market today, but as producers continue to reduce process costs, the nutrients are often under-dosed.

It would, therefore, be highly desirable to be provided with an improved fermentation process that includes yeast nutrients as well as enzymes for supporting the production of fermentation products.

### BRIEF SUMMARY

The present disclosure provides inactivated yeasts and products derived therefrom (which can comprise heterologous enzymes) for improving the yield of a fermentation conducted by a fermenting yeast cell. The yeasts and associated yeast products can be included in a liquefaction medium. The yeast products can be included in a liquefied medium or in a fermentation medium. The yeasts products comprise a source of nutrients for the fermenting organism as well as, in some embodiments, a source of enzyme for facilitating the degradation of the biomass and the conversion of the biomass into a fermentation product (such as, for example, ethanol).

According to a first aspect, the present disclosure provides a process for improving the yield of a fermentation product made from a fermenting yeast cell in a fermenting medium. The process comprises (i) liquefying a liquefaction medium to obtain a fermentation medium; and/or (ii) fermenting the fermentation medium (which can optionally be liquefied) with the fermenting yeast cell to obtain the fermentation product. The process can further comprises including a first inactivated yeast product made from a first recombinant yeast host cell in the liquefaction medium and/or the fermentation medium, wherein the first recombinant yeast host cell comprises a first heterologous nucleic acid molecule for expressing a first heterologous enzyme and the first inactivated yeast product comprises the first heterologous enzyme. Alternatively or in combination, the process can further comprises including a second recombinant yeast host cell in the liquefaction medium to obtain a second inactivated yeast product in the fermentation medium, wherein the second recombinant yeast host cell comprises a second heterologous nucleic acid molecule for expressing a second heterologous enzyme and the second inactivated yeast product comprises the second heterologous enzyme. Alternatively or in combination, the process can further comprises including a third inactivated yeast product made from a non-genetically modified yeast host cell to the liquefaction medium. The process is conducted so as to improve the yield of the fermentation product (for example when compared to a process lacking including the first inactivated yeast product, the second recombinant yeast host cell or the third inactivated yeast product). In an embodiment, the first inactivated yeast product, the second inactivated yeast product and/or the third inactivated yeast product is a yeast extract. In another embodiment, the process can further comprise bead milling, bead beating and/or high pressure homogenizing the first recombinant yeast host cell and/or the non-genetically modified yeast host cell to obtain the first inactivated yeast product and/or the third inactivated yeast product. In some embodiments, the second heterologous nucleic acid molecule allows the intracellular expression of the heterologous enzyme. In some additional embodiments, the second recombinant yeast host cell is provided as a cream yeast. In some alternative embodiments, the first and/or second heterologous nucleic acid molecule allows the expression of the first and/or second heterologous enzyme in association with the membrane of the first and/or second recombinant yeast host cell, such as, for example, in a tethered form. In further embodiments, the first and/or second heterologous nucleic acid molecule allows the expression of the first and/or second heterologous enzyme in a secreted form. In some embodiments, the first and/or second heterologous nucleic acid molecule is operatively associated with a first and/or second promoter allowing the expression of the heterologous enzyme during the propagation of the first and/or second recombinant yeast host cell. In an embodiment, the first and/or second heterologous enzyme can be an amylolytic enzyme. For example, the amylolytic has alpha-amylase activity and can comprise, in some embodiments, the amino acid sequence of any one of SEQ ID NO: 13, 60, 61, 62, 63, or 64; be a variant of the amino acid sequence of any one of SEQ ID NO: 13, 60, 61, 62, 63, or 64; or be a fragment of the amino acid sequence of any one of SEQ ID NO: 13, 60, 61, 62, 63, or 64. In another embodiment, the amylolytic enzyme has glucoamylase activity and can comprise, in some embodiments, the amino acid sequence of SEQ ID NO: 3 or 67; be a variant of the amino acid sequence of SEQ ID NO: 3 or 67; or be a fragment of the amino acid sequence of SEQ ID NO: 3 or 67. In yet a further example, the amylolytic enzyme has trehalase activity and can comprise, in some embodiments, the amino acid sequence of SEQ ID NO: 70 or 71; be a variant of the amino acid sequence of SEQ ID NO: 70 or 71; or be a fragment of the amino acid sequence of SEQ ID NO: 70 or 71. In still another example, the amylolytic enzyme has xylanase activity and can comprise, in some embodiments, the amino acid sequence of SEQ ID NO: 72, be a variant of the amino acid sequence of SEQ ID NO: 72, or be a fragment of the amino acid sequence of SEQ ID NO: 72. In a further embodiment, the first and/or second heterologous enzyme is an esterase. For example, the esterase has phytase activity and can comprise, in some embodiments, the amino acid sequence of SEQ ID NO: 73, be a variant of the amino acid sequence of SEQ ID NO: 73, or be a fragment of the amino acid sequence of SEQ ID NO: 73. In another embodiment, the first and/or second heterologous enzyme is a protease. For example, the protease has aspartic protease activity and can have, in some embodiments, the amino acid sequence of SEQ ID NO: 74 or 75; be a variant of the amino acid sequence of SEQ ID NO: 74 or 75; or be a fragment of the amino acid sequence of SEQ ID NO: 74 or 75. In another embodiment, the fermenting yeast cell is a recombinant fermenting yeast host cell. In some embodiments, the fermenting yeast host cell can comprises a genetic modification for reducing the production of one or more native enzymes that function to produce glycerol or regulate glycerol synthesis, a genetic modification for allowing the production of a second polypeptide having glucoamylase activity, and/or a genetic modification for reducing the production of one or more native enzymes that function to catabolize formate. In some embodiments, the fermenting yeast host cell comprises the genetic modification for allowing the production of the second polypeptide having glucoamylase activity. In embodiment, step (ii) of the process is conducted under anaerobic conditions. In some embodiments, the fermenting medium comprises or is derived from corn, sugar cane or a lignocellulosic material. In additional embodiments, the fermentation product is ethanol. In some embodiment, the process can further comprise including an exogenous polypeptide having alpha-amylase activity with the third inactivated yeast product. In yet another embodiment, the process can comprise including at least 0.00001 g of the first and/or the third inactivated yeast product per L of the fermentation medium. In still another embodiment, the process can be used for increasing the dextrose equivalent and/or the free amino nitrogen of the fermentation medium when compared to the dextrose equivalent and/or the free amino nitrogen of the liquefaction medium.

According to a second aspect, the present disclosure provides an additive for improving the yield of a fermentation product made by a fermenting yeast cell. The additive comprises an inactivated yeast product made from the first recombinant yeast host cell described herein. The additive can be a bead-milled, a beat-beaten or a high pressure homogenized yeast product. The first recombinant yeast host cell comprises the first heterologous nucleic acid molecule for expressing a first heterologous enzyme and the first inactivated yeast product comprises the first heterologous enzyme. In another embodiment, the first heterologous nucleic acid molecule allows the intracellular expression of the first heterologous enzyme. In a further embodiment, the first heterologous nucleic acid molecule allows the expression of the first heterologous enzyme in association with the membrane of the first recombinant yeast host cell. For example, the first heterologous second nucleic acid molecule can allow the expression of the first heterologous enzyme tethered to the membrane of the first recombinant yeast host cell. In still another example, the first heterologous second nucleic acid molecule can allow the expression of the first heterologous enzyme in a secreted form. In yet another embodiment, the first heterologous nucleic acid molecule is operatively associated with a first promoter allowing the expression of the heterologous enzyme during the propagation of the second recombinant yeast host cell. Embodiments of the heterologous enzyme and of the fermenting yeast host cell described herein can be used in the additive.

According to a third aspect, the present disclosure concerns a kit for improving the yield of a fermentation product made from a fermenting yeast cell, the kit comprising (i) at least one component of a liquefaction medium and/or fermentation medium, and (ii) at least one of the first inactivated yeast product, the second recombinant yeast host cell or the third inactivated yeast product as defined herein. In some embodiments, the first and/or third inactivated yeast product is formulated to be added to the liquefaction medium and/or the fermentation medium at a concentration of at least about 0.00001 g/L. In some embodiments, the at least one component can be a carbohydrate source, a phosphorous source and/or a nitrogen source In other embodiments, the kit can further comprise the fermenting yeast cell as defined herein.

According to a fourth aspect, the present disclosure provides a liquefaction medium comprising the first inactivated yeast product, the second recombinant yeast host cell and/or the third inactivated yeast product as described herein.

According to a fifth aspect, the present disclosure provides a fermentation medium comprising the first inactivated yeast product, the second inactivated yeast product and/or the third inactivated yeast product as described herein.

According to a sixth aspect, the present disclosure comprises a process for improving the yield of a fermentation product made from a fermenting yeast cell in a fermenting medium. The process can comprise contacting the first, second and/or third inactivated yeast product described herein with the fermenting yeast cell in the fermentation medium so as to improve the yield of the fermentation product. Alternatively or in combination, the process can comprise adding the second recombinant yeast host cell to the liquefaction medium to obtain a supplemented liquefaction medium and heating (e.g., liquefying) the supplemented liquefaction medium until the second inactive yeast product is obtained. In some embodiments, the fermentation product is ethanol. In still another embodiment, the fermenting medium comprises or is derived from corn, sugar cane or a lignocellulosic material. In a further embodiment, the process can further comprise adding the first, second and/or third inactivated yeast product prior to, at the same time and/or after the fermenting yeast cell is added to the fermentation medium. In another embodiment, the process can comprise adding at least 0.00001 g of the first, second and/or third inactivated yeast product per L of the fermentation medium.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 shows a dextrose equivalent profile associated with the M15958 strain during a laboratory scale fermentation. Results are shown as the percentage of dextrose equivalent in function of time (minutes).

FIG. 2 shows the growth curve of the M11589 strain in Verduyn media in the absence (0 g/L, ⋄) or presence (0.05 g/L (Δ), 0.1 g/L ( [symbol] ) or 0.5 g/L (□)) of a commercial yeast extract. Results are shown as the optical density as measured at 600 nm in function of time (hh:mm) and the concentration of the yeast extract.

FIG. 3 shows the ethanol and glycerol production of the M2390, M8841 or M11589 strains cultured in Verduyn medium for 24 h in the absence (0.00 g/L) or presence (0.01 g/L, 0.1 g/L or 0.5 g/L) of a commercial yeast extract. Results are shown as ethanol concentration (left Y axis, black bars, in g/L) and glycerol concentration (right Y axis, gray squares, in g/L) in function of the yeast strain and the concentration of the yeast extract.

FIG. 4 shows the dry cell weight (DCW) of the M2390, M8841 or M11589 strains cultured in Verduyn medium for 24 h in the absence (0.00 g/L) or presence (0.01 g/L, 0.1 g/L or 0.5 g/L) of a commercial yeast extract. Results are shown as the dry cell weight (in g/L), in function of the yeast strain and the concentration (in g of DCW per L) of the yeast extract.

FIG. 5 shows a growth curve of the M2390 yeast strain cultured in Verduyn medium in the absence (0 g/L) or presence (0.01 g/L, 0.1 g/L or 0.5 g/L) of a commercial yeast extract. Results are shown as the pressure sum (PSI), in function of the concentration of the yeast extract and time.

FIG. 6 shows a growth curve of the M8841 yeast strain cultured in Verduyn medium in the absence (0 g/L) or presence (0.01 g/L, 0.1 g/L or 0.5 g/L) of a commercial yeast extract. Results are shown as the pressure sum (PSI), in function of the concentration of the yeast extract and time.

FIG. 7 shows a growth curve of the M11589 yeast strain cultured in Verduyn medium in the absence (0 g/L) or presence (0.01 g/L, 0.1 g/L or 0.5 g/L) of a commercial yeast extract cultured. Results are shown as the pressure sum (PSI), in function of the concentration of the yeast extract and time.

FIG. 8 shows the fermentation performance of the M2390 strain in a 33% solids fermentation using lab-scale liquefactions supplemented with a commercial alpha-amylase enzyme (0.02% commercial AA); or 0.012%, 0.03%, or 0.3% inactivated yeast (obtained from the M10474 strain) along with a 0.02% commercial alpha-amylase. Results are shown as ethanol concentration (left Y axis, bars, in g/L) and residual glucose (right Y axis, circles ●, in g/L) as a function of the liquefaction conditions.

FIG. 9 shows the fermentation performance of the M2390 strain in a 32% solids fermentation using lab-scale liquefactions supplemented with a commercial alpha-amylase (0.02% commercial alpha-amylase enzyme only); or 0.01%, 0.02%, or 0.03% inactivated yeast (obtained from the M10474 strain) along with a 0.02% commercial alpha-amylase. Results are shown as ethanol concentration (left Y axis, bars, in g/L) and residual glucose (right Y axis, squares ▪, in g/L) or glycerol production (right Y axis, triangles ▴, in g/L) as a function of the liquefaction conditions.

FIG. 10 shows the free amino nitrogen concentrations after liquefaction supplemented with a control commercial alpha-amylase (0.02% commercial alpha-amylase enzyme only) or with a dry cell weight (DCW) additions (0.01%, 0.02%, or 0.03%) of strain M10474. The total soluble nitrogen is shown as free amino nitrogen (FAN) in parts per million (ppm) as a function of the individual liquefaction conditions.

FIG. 11 shows the torque trend profile of lab-scale liquefactions containing: 0.03% g DCW/g solids additions of YPD propped, bead milled inactivated alpha-amylase expressing yeast, M19211, with a 25% (0.005%) dose of commercial alpha-amylase enzyme #1 (▴); 0.03% g DCW/g solids additions of washed high pressure homogenization inactivated alpha-amylase expressing yeast, M19211, with a 25% (0.005%) dose of commercial alpha-amylase enzyme #1 (●); 0.03% g DCW/g solids additions of unwashed high pressure homogenization inactivated alpha-amylase expressing yeast, M19211, with a 25% (0.005%) dose of commercial alpha-amylase enzyme #1 (▪); commercial alpha-amylase enzymes #1 dosed at 100% (0.02% w/w) (dark dashed line); or commercial alpha-amylase enzymes #2 dosed at 100% (0.02% w/w) (light dashed line). Results are shown as torque trends in Newton Centimeters (left Y axis) as a function of time (h:mm:ss, X axis).

FIG. 12 shows the endpoint dextrose equivalent profile of a lab-scale liquefaction containing: 0.03% g DCW/g solids additions of YPD propped, bead milled inactivated alpha-amylase expressing yeast, M19211, with a 25% (0.005%) dose of commercial alpha-amylase enzyme #1; 0.03% gDCW/g solids additions of washed high pressure homogenization inactivated alpha-amylase expressing yeast, M19211, with a 25% (0.005%) dose of commercial alpha-amylase enzyme #1; 0.03% gDCW/g solids additions of unwashed high pressure homogenization inactivated alpha-amylase expressing yeast, M19211, with a 25% (0.005%) dose of commercial alpha-amylase enzyme #1; commercial alpha-amylase enzymes #1 dosed at 100% (0.02% w/w) (dark dashed line); or commercial alpha-amylase enzymes #1 dosed at 100% (0.02% w/w) (light dashed line). Results are shown as % dextrose equivalent (Y axis, gray bars) as a function of the liquefaction conditions.

FIG. 13 shows the potential ethanol obtained using the M2390 strain in a 33% solids fermentation using lab-scale liquefactions dosed with: commercial alpha-amylase enzyme #2 (0.02% w/w); commercial alpha-amylase enzyme #1 (0.02% w/w); 0.03% g DCW/g solids additions of YPD propped, bead milled inactivated alpha-amylase expressing yeast, M19211, with a 25% (0.005%) dose of commercial alpha-amylase enzyme #1; 0.03% g DCW/g solids additions of washed high pressure homogenization inactivated alpha-amylase expressing yeast, M19211, with a 25% (0.005%) dose of commercial alpha-amylase enzyme #1; or 0.03% g DCW/g solids additions of unwashed high pressure homogenization inactivated alpha-amylase expressing yeast, M19211, with a 25% (0.005%) dose of commercial alpha-amylase enzyme #1. Results are shown as potential ethanol concentration (left Y axis, bars, in g/L) as a function of the liquefaction conditions.

FIG. 14 shows fermentation performance of various yeast strains in a 32% solids fermentation using nutrient rich commercial mash. Percentage exogenous glucoamylase (“% GA”) refers to percentage dose of commercial glucoamylase used during the fermentation. Results are shown as ethanol concentrations (left Y axis, bars, in g/L), residual glucose (right Y axis, circles ●, in g/L), and glycerol (right Y axis, triangles ▴, in g/L) as a function of the inactivated yeast addition and respective exogenous GA dose.

FIG. 15 shows fermentation performance of various yeast strains in a 30% solids fermentation using nutrient poor commercial mash. Results are shown as ethanol concentrations (left Y axis, gray bars, in g/L), residual glucose (right Y axis, black circles, in g/L), and glycerol (right Y axis, black triangles, in g/L) as a function of the inactivated yeast addition.

FIG. 16 shows the torque trend profile of lab-scale liquefactions containing: commercial alpha-amylases enzyme #1 dosed at 100% (0.02% w/w) (dark dashed line); commercial alpha-amylases enzyme #2 dosed at 100% (0.02% w/w) (light dashed line); autolysized strain M19211 dosed at 0.03% g DCW/g solids additions of inactivated alpha-amylase expressing yeast, with a 25% (0.005%) dose of commercial alpha-amylase enzyme #1 (□); bead beaten or milled strain M19211 dosed at 0.03% g DCW/g solids additions of inactivated alpha-amylase expressing yeast, with a 25% (0.005%) dose of commercial alpha-amylase enzyme #1 (□); or high pressure homogenized strain M19211 dosed at 0.03% g DCW/g solids additions of inactivated alpha-amylase expressing yeast, with a 25% (0.005%) dose of commercial alpha-amylase enzyme #1 (□). Results shown as torque trends in Newton Centimeters (Y axis) as a function of time (X-axis, h:mm:ss).

FIG. 17 shows the endpoint dextrose equivalent of a lab-scale liquefaction containing: autolysized strain M19211 dosed at 0.03% g DCW/g solids additions of inactivated alpha-amylase expressing yeast, with a 25% (0.005%) dose of commercial alpha-amylase enzyme #1 (autolysis 0.003% DCW M19211+0.0005% commercial alpha-amylase enzyme #1); bead beaten or milled strain M19211 dosed at 0.03% g DCW/g solids additions of inactivated alpha-amylase expressing yeast, with a 25% (0.005%) dose of commercial alpha-amylase enzyme #1 (bead milled 0.003% DCW M19211+0.005% commercial alpha-amylase enzyme #1); high pressure homogenized strain M19211 dosed at 0.03% g DCW/g solids additions of inactivated alpha-amylase expressing yeast, with a 25% (0.005%) dose of commercial alpha-amylase enzyme #1 (high pressure homogenization 0.03% DCW M19211+0.005% commercial alpha-amylase enzyme #1); commercial alpha-amylase enzyme #1 dosed at 100% (0.02% w/w, commercial alpha-amylase enzyme #1); or commercial alpha-amylase enzyme #2 dosed at 100% (0.02% w/w, commercial alpha-amylase enzyme #2). Results are shown as % dextrose equivalent (Y axis) as a function of the liquefaction conditions (X axis).

FIG. 18 shows the dextrose equivalent profile of a 1 g mini-liquefaction hydrolyzed with various M19211 inactivation methods: cream unwashed, cream washed, bead milled unwashed, high pressure homogenized unwashed, high pressure homogenized washed, instant dry yeast (IDY) unwashed, IDY washed, YPD unprocessed, and YPD bead beaten. Results are shown as % dextrose equivalent (Y axis) as a function of inactivation methods (X axis).

### DETAILED DESCRIPTION

In accordance with an aspect of the present disclosure, there is provided additives (in the form of a recombinant yeast host cell or in the form of an inactivated yeast product) for improving the yield of a fermentation product made by a fermenting yeast cell. As used in the present disclosure, the expression “additive” refers to a product that supplies nutrients (such as, for example, a nitrogen source) for purposes of improving an organism's performance (e.g., providing enhanced robustness in a harsh and/or variable conditions, such as in fermentation). The additive includes a yeast product, which can be an inactivated yeast product (such as, for example, a yeast extract) made from a non-genetically modified yeast cell and/or a recombinant yeast host cell. The recombinant yeast host cell includes an heterologous nucleic acid molecule for expressing an heterologous enzyme (which is present in the yeast product).

As used in the context of the present disclosure, a “yeast product” is a product obtained from a yeast cell (which may be genetically modified or not). When the yeast product is made from a recombinant yeast host cell, it comprises the heterologous enzyme (encoded by the heterologous nucleic acid molecule).

The yeast product can be an active or semi-active product, such as, for example, a cream yeast or propped yeast cell. The yeast product can be, for example, an inactivated whole cell yeast, a yeast lysate (e.g., an autolysate), a yeast extract, and/or a yeast fraction (e.g., yeast cell walls). The yeast extract can be a bead-milled yeast extract obtained from bead milling the yeast cell. The yeast extract can be a bead-beaten yeast extract obtained from bead beating the yeast cell. The yeast extract can be a high pressure homogenized yeast extract obtained from high pressure homogenizing the yeast cell. The yeast product can be made prior to the beginning of the liquefaction and/or fermentation by means known to those skilled in the art. Alternatively or in combination, the yeast product can be made in situ prior to fermentation (for example during liquefaction) or during the fermentation by adding the second recombinant yeast host cell to the fermentation medium and treating the fermentation medium (for example by using heat) to convert the recombinant yeast host cell into a yeast product.

The additive includes nutrients that supports the growth and/or viability of the fermenting yeast cell; improve the fermenting yeast cell's robustness; and/or increase the likelihood that the fermenting yeast cell will survive fermentation conditions, such as high ethanol and/or reducing sugars, fluctuating temperatures, and/or presence of organic acids from contamination events. As shown in the examples below, the additive can be used to improve the liquefaction step by increasing the dextrose equivalent and/or the free amino acid content of the liquefied fermentation method and/or reduce the need for adding purified enzyme during the liquefaction step. The cost of preparing a yeast product from the second recombinant yeast host cell may be similar to that of conventional yeast extracts. However, since the recombinant yeast host cell expresses the heterologous enzyme, which is present in the yeast product, the yeast product can provide additional functionality not present in conventional yeast extracts.

### Non Genetically Modified Yeast Cells

In some embodiments, the yeast cells used to provide the yeast product are not genetically modified, e.g., they do not include genetic modifications introduced purposively by a human and are not the progeny of yeast host cells which have been genetically modified. Suitable non-genetically modified yeast host cells that can be used in the context of the present disclosure to make the first additive can be, for example, from the genus Saccharomyces, Kluyveromyces, Arxula, Debaryomyces, Candida, Pichia, Phaffia, Schizosaccharomyces, Hansenula, Kloeckera, Schwanniomyces, Torula or Yarrowia. Suitable yeast species can include, for example, S. cerevisiae, S. bulderi, S. barnetti, S. exiguus, S. uvarum, S. diastaticus, S. boulardii, C. utilis, K. lactis, K. marxianus or K. fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some further embodiments, the yeast is from Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe or Schwanniomyces occidentalis. In one particular embodiment, the yeast host cell is Saccharomyces cerevisiae. In some embodiments, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the host cell can be an oleaginous microalgae host cell (e.g., for example, from the genus Thraustochytrium or Schizochytrium). In an embodiment, the yeast cell and the recombinant yeast host cell are from the genus Saccharomyces and, in some embodiments, from the species Saccharomyces cerevisiae.

### Recombinant Yeast Host Cells

In some embodiments, the yeast host cells are recombinant yeast host cells that have been genetically engineered. The genetic modification(s) is (are) aimed at increasing the expression of a specific targeted gene (which is considered heterologous to the yeast host cell) and can be made in one or multiple (e.g., 1, 2, 3, 4, 5, 6, 7, 8 or more) genetic locations. In the context of the present disclosure, when recombinant yeast cell is qualified as being “genetically engineered”, it is understood to mean that it has been manipulated to add at least one or more heterologous or exogenous nucleic acid residue (e.g., a genetic modification). In some embodiments, the one or more nucleic acid residues that are added can be derived from an heterologous cell or the recombinant host cell itself. In the latter scenario, the nucleic acid residue(s) is (are) added at one or more genomic location which is different than the native genomic location. The genetic manipulations did not occur in nature and are the results of in vitro manipulations of the yeast.

When expressed in recombinant yeast host cells, the heterologous enzymes described herein are encoded on one or more heterologous nucleic acid molecules. The term “heterologous” when used in reference to a nucleic acid molecule (such as a promoter, a terminator or a coding sequence) or a protein (such as an enzyme) refers to a nucleic acid molecule or a protein that is not natively found in the recombinant host cell. “Heterologous” also includes a native coding region/promoter/terminator, or portion thereof, that is introduced into the source organism in a form that is different from the corresponding native gene, e.g., not in its natural location in the organism's genome. The heterologous nucleic acid molecule is purposively introduced into the recombinant host cell. For example, a heterologous element could be derived from a different strain of host cell, or from an organism of a different taxonomic group (e.g., different domain, kingdom, phylum, class, order, family genus, or species, or any subgroup within one of these classifications).

The heterologous nucleic acid molecule present in the recombinant yeast host cell can be integrated in the host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies (e.g., 2, 3, 4, 5, 6, 7, 8 or even more copies) in the yeast host cell's genome. Alternatively, the heterologous nucleic acid molecule can be independently replicating from the yeast's genome. In such embodiment, the nucleic acid molecule can be stable and self-replicating.

Suitable recombinant yeast host cells that can be used in the context of the present disclosure can be, for example, from the genus Saccharomyces, Kluyveromyces, Arxula, Debaryomyces, Candida, Pichia, Phaffia, Schizosaccharomyces, Hansenula, Kloeckera, Schwanniomyces, Torula or Yarrowia. Suitable yeast species can include, for example, S. cerevisiae, S. bulderi, S. barnetti, S. exiguus, S. uvarum, S. diastaticus, S. boulardii, C. utilis, K. lactis, K. marxianus or K. fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In a further embodiment, the recombinant yeast host cell is from Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe or Schwanniomyces occidentalis In one particular embodiment, the yeast host cell is Saccharomyces cerevisiae. In some embodiments, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the host cell can be an oleaginous microalgae host cell (e.g., for example, from the genus Thraustochytrium or Schizochytrium). The yeast cell and the recombinant yeast host cell can be from the same or different genus or species. In an embodiment, the recombinant yeast host cell is from the genus Saccharomyces and, in some embodiments, from the species Saccharomyces cerevisiae. In an embodiment, the yeast cell and the recombinant yeast host cell are from the genus Saccharomyces and, in some embodiments, from the species Saccharomyces cerevisiae.

### Heterologous Enzyme

The recombinant yeast host cell of the present disclosure includes an heterologous nucleic acid molecule intended to allow the expression (e.g., encoding) of one or more heterologous enzymes. In an embodiment, the recombinant yeast host cell can include more than one heterologous nucleic acid molecules for expressing more than one heterologous enzymes. In some specific embodiments, the recombinant yeast host cell can include express two distinct heterologous enzymes which can be encoded on one or more heterologous nucleic acid molecules. In the context of the present disclosure, the heterologous enzyme can be, without limitation, an enzyme involved in the cleavage or hydrolysis of its substrate (e.g., a lytic enzyme and, in some embodiments, a saccharolytic enzyme). In still another embodiment, the enzyme can be a glycoside hydrolase. In the context of the present disclosure, the term “glycoside hydrolase” refers to an enzyme involved in carbohydrate digestion, metabolism and/or hydrolysis, including amylases, cellulases, hemicellulases, cellulolytic and amylolytic accessory enzymes, inulinases, levanases, trehalases, pectinases, and pentose sugar utilizing enzymes. In another embodiment, the enzyme can be a protease. In the context of the present disclosure, the term “protease” refers to an enzyme involved in protein digestion, metabolism and/or hydrolysis. In yet another embodiment, the enzyme can be an esterase. In the context of the present disclosure, the term “esterase” refers to an enzyme involved in the hydrolysis of an ester from an acid or an alcohol, including phosphatases such as phytases.

As used in the context of the present disclosure, the expression “hydrolase” (E.C. 3) refers to a protein having enzymatic activity and capable of catalyzing the hydrolysis of a chemical bound. For example, the hydrolase can be an esterase (E.C. 3.1 for example phytase, lipase, phospholipase A1 and/or phospholipase A2), can cleaved C-N non-peptide bonds (E.C. 3.5 for example an asparaginase), can be a glycosylase (E.C. 3.2 for example an amylase (E.C. 3.2.1.1), a glucanase, a glycosidase (E.C. 3.2.1), a cellulase (E.C. 3.2.1.4), a trehalase (E.C. 3.2.1.28), a pectinase and/or a lactase (E.C. 3.2.1.108)), a protease (E.C. 3.4 for example a bacterial protease, a plant protease or a fungal protease). When the hydrolase is an amylase, it can be, for example, a fungal alpha amylase, a bacterial alpha amylase, a maltogenic alpha amylase, a maltotetrahydrolase, a plant (e.g., barley) alpha or beta amylase, a fungal alpha amylase and/or a glucoamylase. When the hydrolase is a glycosidase, it can be, for example, a beta glucosidase. When the hydrolase is a cellulase, it can be, for example, a cellulase and/or an hemicellulase (such as, for example, a xylanase).

In some embodiments, the hydrolase is an amylolytic enzyme. As used herein, the expression “amylolytic enzyme” refers to a class of enzymes capable of hydrolyzing starch or hydrolyzed starch. Amylolytic enzymes include, but are not limited to α-amylases (EC 3.2.1.1, sometimes referred to fungal α-amylase, see below), maltogenic amylase (EC 3.2.1.133), glucoamylase (EC 3.2.1.3), glucan 1,4-α-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae (and have, for example, the amino acid sequence of SEQ ID NO: 1, a variant thereof or a fragment thereof), Saccharomycopsis fibuligera (GenBank Accession #CAA29233.1) (and have, for example, the amino acid sequence of SEQ ID NO: 68, a variant thereof or a fragment thereof), and Bacillus amyloliquefaciens (GenBank Accession #ABS72727) (and have, for example, the amino acid sequence of SEQ ID NO: 69, a variant thereof or a fragment thereof); a maltogenic alpha-amylase from Geobacillus stearothermophilus (and have, for example, the amino acid sequence of SEQ ID NO: 2, a variant thereof or a fragment thereof), a glucoamylase from Saccharomycopsis fibuligera (and have, for example, the amino acid sequence of SEQ ID NO: 3, a variant thereof or a fragment thereof), and Rasamsonia emersonii (GenBank Accession #CAC28076) (and have, for example, the amino acid sequence of SEQ ID NO: 67, a variant thereof or a fragment thereof); a glucan 1,4-alpha-maltotetraohydrolase from Pseudomonas saccharophila (and have, for example, the amino acid sequence of SEQ ID NO: 4, a variant thereof or a fragment thereof), a pullulanase from Bacillus naganoensis (and have, for example, the amino acid sequence of SEQ ID NO: 5, a variant thereof or a fragment thereof), a pullulanase from Bacillus acidopullulyticus (and have, for example, the amino acid sequence of SEQ ID NO: 6, a variant thereof or a fragment thereof), an iso-amylase from Pseudomonas amyloderamosa (and have, for example, the amino acid sequence of SEQ ID NO: 7, a variant thereof or a fragment thereof), amylomaltase from Thermus thermophilus (and have, for example, the amino acid sequence of SEQ ID NO: 8, a variant thereof or a fragment thereof), and/or a thermo-tolerant from alpha-amylase from Pyrococcus furiosus (GenBank Accession #WP_014835153.1) (and have, for example, the amino acid sequence of SEQ ID NO: 13 or 64, a variant thereof or a fragment thereof), Thermococcus thioreducens (GenBank Accession #WP_055428342.1) (and have, for example, the amino acid sequence of SEQ ID NO: 10 or 61, a variant thereof or a fragment thereof), Thermococcus eurythermalis ; (GenBank Accession #WP_050002265.1) (and have, for example, the amino acid sequence of SEQ ID NO: 11 or 62, a variant thereof or a fragment thereof), Thermococcus hydrothermalis (GenBank Accession #AAC97877.1) (and have, for example, the amino acid sequence of SEQ ID NO: 12 or 63, a variant thereof or a fragment thereof), and Thermococcus gammatolerans (GenBank Accession #ACS32724.1) (and have, for example, the amino acid sequence of SEQ ID NO: 9 or 60, a variant thereof or a fragment thereof). In an embodiment, the heterologous enzyme is an alpha-amylase from Pyrococcus furiosus (GenBank Accession #WP_014835153.1) (and have, for example, the amino acid sequence of SEQ ID NO: 13, a variant thereof or a fragment thereof). In an embodiment, the heterologous enzyme is derived from a Pyrococcus furiosus alpha amylase (and have, for example, the amino acid sequence of SEQ ID NO: 65, a variant thereof or a fragment thereof). In an embodiment, the heterologous enzyme is derived from a Thermococcus hydrothermalis alpha amylase (and have, for example, the amino acid sequence of SEQ ID NO: 66, a variant thereof or a fragment thereof).

In some embodiments, the hydrolase is a trehalase enzyme. As used herein, the expression “trehalase enzyme” refers to a class of enzymes capable of catalyzing the conversion of trehalose to glucose. In an embodiment, the one or more trehalase enzymes can be a trehalase from Aspergillus fumigatus (GenBank Accession #XP_748551) (and have, for example, the amino acid sequence of SEQ ID NO: 70, a variant thereof or a fragment thereof), and Neurospora crassa (GenBank Accession #XP_960845.1) (and have, for example, the amino acid sequence of SEQ ID NO: 71, a variant thereof or a fragment thereof).

The additional heterologous enzyme can be a “cellulolytic enzyme”, an enzyme involved in cellulose digestion, metabolism and/or hydrolysis. The term “cellulase” refers to a class of enzymes that catalyze cellulolysis (i.e. the hydrolysis of cellulose). Several different kinds of cellulases are known, which differ structurally and mechanistically. There are general types of cellulases based on the type of reaction catalyzed: endocellulase breaks internal bonds to disrupt the crystalline structure of cellulose and expose individual cellulose polysaccharide chains; exocellulase cleaves 2-4 units from the ends of the exposed chains produced by endocellulase, resulting in the tetrasaccharides or disaccharide such as cellobiose. There are two main types of exocellulases (or cellobiohydrolases, abbreviate CBH)—one type working processively from the reducing end, and one type working processively from the non-reducing end of cellulose; cellobiase or beta-glucosidase hydrolyses the exocellulase product into individual monosaccharides; oxidative cellulases that depolymerize cellulose by radical reactions, as for instance cellobiose dehydrogenase (acceptor); cellulose phosphorylases that depolymerize cellulose using phosphates instead of water. In the most familiar case of cellulase activity, the enzyme complex breaks down cellulose to beta-glucose. A “cellulase” can be any enzyme involved in cellulose digestion, metabolism and/or hydrolysis, including an endoglucanase, glucosidase, cellobiohydrolase, xylanase, glucanase, xylosidase, xylan esterase, arabinofuranosidase, galactosidase, cellobiose phosphorylase, cellodextrin phosphorylase, mannanase, mannosidase, xyloglucanase, endoxylanase, glucuronidase, acetylxylanesterase, arabinofuranohydrolase, swollenin, glucuronyl esterase, expansin, pectinase, and feruoyl esterase protein.

The additional heterologous enzyme can have “hemicellulolytic activity”, an enzyme involved in hemicellulose digestion, metabolism and/or hydrolysis. The term “hemicellulase” refers to a class of enzymes that catalyze the hydrolysis of cellulose. Several different kinds of enzymes are known to have hemicellulolytic activity including, but not limited to, xylanases and mannanases.

The additional heterologous enzyme can have “xylanolytic activity”, an enzyme having the is ability to hydrolyze glycosidic linkages in oligopentoses and polypentoses. The term “xylanase” is the name given to a class of enzymes which degrade the linear polysaccharide beta-1,4-xylan into xylose, thus breaking down hemicellulose, one of the major components of plant cell walls. Xylanases include those enzymes that correspond to Enzyme Commission Number 3.2.1.8. The heterologous protein can also be a “xylose metabolizing enzyme”, an enzyme involved in xylose digestion, metabolism and/or hydrolysis, including a xylose isomerase, xylulokinase, xylose reductase, xylose dehydrogenase, xylitol dehydrogenase, xylonate dehydratase, xylose transketolase, and a xylose transaldolase protein. A “pentose sugar utilizing enzyme” can be any enzyme involved in pentose sugar digestion, metabolism and/or hydrolysis, including xylanase, arabinase, arabinoxylanase, arabinosidase, arabinofuranosidase, arabinoxylanase, arabinosidase, and arabinofuranosidase, arabinose isomerase, ribulose-5-phosphate 4-epimerase, xylose isomerase, xylulokinase, xylose reductase, xylose dehydrogenase, xylitol dehydrogenase, xylonate dehydratase, xylose transketolase, and/or xylose transaldolase. In an embodiment, the one or more xylanase enzymes can be a xylanase from Aspergillus niger (GenBank Accession #CAA03655.1) (and have, for example, the amino acid sequence of SEQ ID NO: 72, a variant thereof or a fragment thereof).

The additional heterologous enzyme can have “mannanic activity”, an enzyme having the is ability to hydrolyze the terminal, non-reducing β-D-mannose residues in β-D-mannosides. Mannanases are capable of breaking down hemicellulose, one of the major components of plant cell walls. Xylanases include those enzymes that correspond to Enzyme Commission Number 3.2.25.

The additional heterologous enzyme can be a “pectinase”, an enzyme, such as pectolyase, pectozyme and polygalacturonase, commonly referred to in brewing as pectic enzymes. These enzymes break down pectin, a polysaccharide substrate that is found in the cell walls of plants.

The additional heterologous enzyme can have “phytolytic activity”, an enzyme catalyzing the conversion of phytic acid into inorganic phosphorus. Phytases (EC 3.2.3) can be belong to the histidine acid phosphatases, β-propeller phytases, purple acid phosphastases or protein tyrosine phosphatase-like phytases family. In an embodiment, the one or more phytase enzymes can be a phytase from Citrobacter braakii (GenBank Accession #AY471611.1) (and have, for example, the amino acid sequence of SEQ ID NO: 73, a variant thereof or a fragment thereof).

The additional heterologous enzyme can have “proteolytic activity”, an enzyme involved in protein digestion, metabolism and/or hydrolysis, including serine proteases, threonine proteases, cysteine proteases, aspartate proteases (e.g., proteases having aspartic activity), glutamic acid proteases and metalloproteases. In some embodiments, the heterologous enzyme having proteolytic activity is a protease enzyme. In an embodiment, the one or more protease enzymes can be a protease from Saccharomycopsis fibuligera (GenBank Accession #P22929) (and have, for example, the amino acid sequence of SEQ ID NO: 74, a variant thereof or a fragment thereof), and Aspergillus fumigatus (GenBank Accession #P41748) (and have, for example, the amino acid sequence of SEQ ID NO: 75, a variant thereof or a fragment thereof).

The heterologous enzyme can be a variant of a known/native enzyme. A variant comprises at least one amino acid difference when compared to the amino acid sequence of the native/know enzyme. As used herein, a variant refers to alterations in the amino acid sequence that do not adversely affect the biological functions of the heterologous enzyme. A substitution, insertion or deletion is said to adversely affect the enzyme when the altered sequence prevents or disrupts a biological function associated with the heterologous enzyme. For example, the overall charge, structure or hydrophobic-hydrophilic properties of the enzyme can be altered without adversely affecting a biological activity. Accordingly, the amino acid sequence can be altered, for example to render the peptide more hydrophobic or hydrophilic, without adversely affecting the biological activities of the heterologous enzyme. The enzyme variants have at least 50%, 55%, 60%, 65%, 70%, 75%, 80%, 85%, 90%, 95%, 96%, 97%, 98% or 99% identity to the heterologous enzyme described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant heterologous enzyme described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A “variant” of the heterologous enzyme can be a conservative variant or an allelic variant.

The heterologous enzyme can be a fragment of a known/native enzyme or fragment of a variant of a known/native enzyme. In some embodiments, the fragment corresponds to the known/native enzyme to which the signal peptide has been removed. In additional embodiments, heterologous protein “fragments” have at least at least 100, 200, 300, 400, 500, 600, 700, 800, 900 or more consecutive amino acids of the heterologous enzyme. A fragment comprises at least one less amino acid residue when compared to the amino acid sequence of the known/native heterologous enzyme and still possess the enzymatic activity of the full-length heterologous enzyme. In some embodiments, fragments of the heterologous enzyme can be employed for producing the corresponding full-length heterologous by peptide synthesis. Therefore, the fragments can be employed as intermediates for producing the full-length proteins.

In the recombinant yeast host cell of the present disclosure, the heterologous enzyme can be “cell-associated” to the recombinant yeast host cell because it is designed to be expressed and remain physically associated with the recombinant yeast host cells. In an embodiment, the heterologous enzyme can be expressed inside the recombinant yeast host cell (intracellularly). In such embodiment, the heterologous enzyme does not need to be associated to the recombinant yeast host cell's wall. When the heterologous enzyme is intended to be expressed intracellularly, its signal peptide, if present in the native sequence, can be deleted to allow intracellular expression.

In another embodiment, the heterologous enzyme of the present disclosure can be secreted. In some embodiments, the secreted heterologous enzyme remains physically associated with the recombinant yeast host cell. In an embodiment, at least one portion (usually at least one terminus) of the heterologous enzyme is bound, covalently, non-covalently and/or electrostatically for example, to cell wall (and in some embodiments to the cytoplasmic membrane). For example, the heterologous enzyme can be modified to bear one or more transmembrane domains, to have one or more lipid modifications (myristoylation, palmitoylation, farnesylation and/or prenylation), to interact with one or more membrane-associated protein and/or to interactions with the cellular lipid rafts. While the heterologous enzyme may not be directly bound to the cell membrane or cell wall (e.g., such as when binding occurs via a tethering moiety), the enzyme is nonetheless considered a “cell-associated” heterologous enzyme according to the present disclosure.

In some embodiments, the heterologous enzyme can be expressed to be located at and associated to the cell wall of the recombinant yeast host cell. In some embodiments, the heterologous enzyme is expressed to be located at and associated to the external surface of the cell wall of the host cell. Recombinant yeast host cells all have a cell wall (which includes a cytoplasmic membrane) defining the intracellular (e.g., internally-facing the nucleus) and extracellular (e.g., externally-facing) environments. The heterologous enzyme can be located at (and in some embodiments, physically associated to) the external face of the recombinant yeast host's cell wall and, in further embodiments, to the external face of the recombinant yeast host's cytoplasmic membrane. In the context of the present disclosure, the expression “associated to the external face of the cell wall/cytoplasmic membrane of the recombinant yeast host cell” refers to the ability of the heterologous enzyme to physically integrate (in a covalent or non-covalent fashion), at least in part, in the cell wall (and in some embodiments in the cytoplasmic membrane) of the recombinant yeast host cell. The physical integration can be attributed to the presence of, for example, a transmembrane domain on the heterologous enzyme, a domain capable of interacting with a cytoplasmic membrane protein on the heterologous enzyme, a post-translational modification made to the heterologous enzyme (e.g., lipidation), etc.

Some heterologous enzymes have the intrinsic ability to locate at and associate to the cell wall of a recombinant yeast host cell (e.g., being cell-associated). Examples of heterologous enzymes having the intrinsic ability of being cell-associated may be found, for example, in PCT Application No. PCT/IB2018/051670 filed on Mar. 13, 2018 and published under WO2018/167669 on Sep. 20, 2018.

However, in some circumstances, it may be warranted to increase or provide cell association to some heterologous enzymes because they exhibit insufficient intrinsic cell association or simply lack intrinsic cell association. In such embodiment, it is possible to provide the heterologous enzyme as a chimeric construct by combining it with a tethering amino acid moiety which will provide or increase attachment to the cell wall of the recombinant yeast host cell. In such embodiment, the chimeric heterologous enzyme will be considered “tethered”. It is preferred that the amino acid tethering moiety of the chimeric enzyme be neutral with respect to the biological activity of the heterologous enzyme, e.g., does not interfere with the enzymatic activity of the heterologous enzyme. In some embodiments, the association of the amino acid tethering moiety with the heterologous enzyme can increase the biological activity of the heterologous enzyme (when compared to the non-tethered, “free” form).

In an embodiment, a tethering moiety can be used to be expressed with the heterologous enzyme to locate the heterologous enzyme to the wall of the recombinant yeast host cell. Various tethering amino acid moieties are known art and can be used in the chimeric enzymes of the present disclosure. The tethering moiety can be a transmembrane domain found on another protein and allow the chimeric enzyme to have a transmembrane domain. In such embodiment, the tethering moiety can be derived from the FLO1 protein (having, for example, the amino acid sequence of SEQ ID NO: 15, a variant thereof or a fragment thereof or being encoded by the nucleic acid sequence of SEQ ID NO: 14).

In still another example, the amino acid tethering moiety can be modified post-translation to include a glycosylphosphatidylinositol (GPI) anchor and allow the chimeric protein to have a GPI anchor. GPI anchors are glycolipids attached to the terminus of a protein (and in some embodiments, to the carboxyl terminus of a protein) which allows the anchoring of the protein to the cytoplasmic membrane of the cell membrane. Tethering amino acid moieties capable of providing a GPI anchor include, but are not limited to those associated with/derived from a SED1 protein (having, for example, the amino acid sequence of SEQ ID NO: 17, a variant thereof or a fragment thereof or being encoded by the nucleic acid sequence of SEQ ID NO: 16), a TIR1 protein (having, for example, the amino acid sequence of SEQ ID NO: 25, a variant thereof or a fragment thereof or being encoded by the nucleic acid sequence of SEQ ID NO: 24), a CWP2 protein (having, for example, the amino acid sequence of SEQ ID NO: 23, a variant thereof or a fragment thereof or being encoded by the nucleic acid sequence of SEQ ID NO: 22), a CCW12 protein (having, for example, the amino acid sequence of SEQ ID NO: 21, a variant thereof or a fragment thereof or being encoded by the nucleic acid sequence of SEQ ID NO: 20), a SPI1 protein (having, for example, the amino acid sequence of SEQ ID NO: 19, a variant thereof or a fragment thereof or being encoded by the nucleic acid sequence of SEQ ID NO: 18), a PST1 protein (having, for example, the amino acid sequence of SEQ ID NO: 27, a variant thereof or a fragment thereof or being encoded by the nucleic acid sequence of SEQ ID NO: 26) or a combination of a AGA1 and a AGA2 protein (having, for example, the amino acid sequence of SEQ ID NO: 29, a variant thereof or a fragment thereof or being encoded by the nucleic acid sequence of SEQ ID NO: 28 or having, for example, the amino acid sequence of SEQ ID NO: 31, a variant thereof or a fragment thereof or being encoded by the nucleic acid sequence of SEQ ID NO: 30).

The tethering amino acid moiety can be a variant of a known/native tethering amino acid moiety, for example a variant of the tethering amino acid moieties described herein. A variant comprises at least one amino acid difference when compared to the amino acid sequence of the native tethering amino acid moiety. As used herein, a variant refers to alterations in the amino acid sequence that do not adversely affect the biological functions of the tethering amino acid moiety (e.g., the ability to locate on the external face and the anchorage of the heterologous protein in the cytoplasmic membrane). A substitution, insertion or deletion is said to adversely affect the protein when the altered sequence prevents or disrupts a biological function associated with the tethering amino acid moiety (e.g., the location on the external face and the anchorage of the heterologous protein in the cytoplasmic membrane). For example, the overall charge, structure or hydrophobic-hydrophilic properties of the protein can be altered without adversely affecting a biological activity. Accordingly, the amino acid sequence can be altered, for example to render the peptide more hydrophobic or hydrophilic, without adversely affecting the biological activities of the tethering amino acid moiety. The tethering amino acid moiety variants have at least 50%, 55%, 60%, 65%, 70%, 75%, 80%, 85%, 90%, 95%, 96%, 97%, 98% or 99% identity to the tethering amino acid moieties described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant tethering amino acid moieties described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. A “variant” of the tethering amino acid moiety can be a conservative variant or an allelic variant.

The tethering amino acid moiety can be a fragment of a known/native tethering amino acid moiety or fragment of a variant of a known/native tethering amino acid moiety. Tethering amino acid moiety “fragments” have at least at least 10, 20, 30, 40, 50, 60, 70, 80, 90, 100 or more consecutive amino acids of the tethering amino acid moiety. A fragment comprises at least one less amino acid residue when compared to the amino acid sequence of the known/native tethering amino acid moiety and still possess the biological activity of the full-length tethering amino acid moiety (e.g., the location to the cell wall).

In embodiments in which an amino acid tethering moiety is desirable, the heterologous enzyme can be provided as a chimeric enzyme expressed by the recombinant yeast host cell and having one of the following formulae (provided from the amino (NH₂) to the carboxyl (COOH) orientation):

HE-L-TT (I)

or

TT-L-HE (II)

In both of these formulae, the residue “HE” refers to the heterologous enzyme moiety, the residue “L” refers to the presence of an optional linker while the residue “TT” refers to an amino acid tethering moiety. In the chimeric enzymes of formula (I), the amino terminus of the amino acid tether is located (directly or indirectly) at the carboxyl (COOH or C) terminus of the heterologous enzyme moiety. In the chimeric enzymes of formula (II), the carboxy terminus of the amino acid tether is located (directly or indirectly) at the amino (NH₂ or N) terminus of the heterologous enzyme moiety.

When the amino acid linker (L) is absent, the tethering amino acid moiety is directly associated with the heterologous enzyme. In the chimeras of formula (I), this means that the carboxyl terminus of the heterologous enzyme moiety is directly associated (with an amide linkage) to the amino terminus of the tethering amino acid moiety. In the chimeras of formula (II), this means that the carboxyl terminus of the tethering amino acid moiety is directly associated (with an amide linkage) to the amino terminus of the heterologous enzyme.

In some embodiments, the presence of an amino acid linker (L) is desirable either to provide, for example, some flexibility between the heterologous enzyme moiety and the tethering amino acid moiety or to facilitate the construction of the heterologous nucleic acid molecule. As used in the present disclosure, the “amino acid linker” or “L” refer to a stretch of one or more amino acids separating the heterologous enzyme moiety HE and the amino acid tethering moiety TT (e.g., indirectly linking the heterologous enzyme HE to the amino acid tethering moiety TT). Amino acid linkers are often composed of flexible residues like glycine and serine so that the adjacent protein domains or polypeptides are free to move relative to one another. Longer linkers are used when it is necessary to ensure that two adjacent domains do not sterically interfere with one another. It is preferred that the amino acid linker be neutral, e.g., does not interfere with the biological activity of the heterologous enzyme nor with the biological activity of the amino acid tethering moiety. In some embodiments, the amino acid linker L can increase the biological activity of the heterologous enzyme moiety and/or of the amino acid tethering moiety.

In instances in which the linker (L) is present in the chimeras of formula (I), its amino end is associated (with an amide linkage) to the carboxyl end of the heterologous enzyme moiety and its carboxyl end is associated (with an amide linkage) to the amino end of the amino acid tethering moiety. In instances in which the linker (L) is present in the chimeras of formula (II), its amino end is associated (with an amide linkage) to the carboxyl end of the amino acid tethering moiety and its carboxyl end is associated (with an amide linkage) to the amino end of the heterologous enzyme moiety.

Various amino acid linkers exist and include, without limitations, (GS)ₙ; (GGS)ₙ; (GGGS)ₙ; (GGGGS)ₙ; (GGSG)ₙ; (GSAT)ₙ, wherein n=is an integer between 1 to 8 (or more). In an embodiment, the amino acid linker L is (GGGGS)ₙ (also referred to as a G₄S motif) and, in still further embodiments, the amino acid linker L comprises more than one G₄S motifs. In some embodiments, L is chosen from: (G4S)₃ (SEQ ID NO: 32), (G)₈ (SEQ ID NO: 33) or (G4S)₈ (SEQ ID NO: 34).

The amino acid linker can also be, in some embodiments, GSAGSAAGSGEF (SEQ ID NO: 35).

Additional amino acid linkers exist and include, without limitations, (EAAK)ₙ and (EAAAK)ₙ, wherein n=is an integer between 1 to 8 (or more). In some embodiments, the one or more (EAAK)ₙ/(EAAAK)ₙ motifs can be separated by one or more additional amino acid residues. In an embodiment, the amino acid linker comprises one or more EA₂K (SEQ ID NO: 49) or EA₃K (SEQ ID NO: 50) motifs. In an embodiment, the amino acid linker can be (EAAK)₃ and has the amino acid sequence of SEQ ID NO: 36. In another embodiment, the amino acid linker can be (A(EAAAK)₄ALEA(EAAAK)₄A) and has the amino acid sequence of SEQ ID NO: 38.

Further amino acid linkers include those having one or more (AP)ₙ motifs wherein n=is an integer between 1 to 10 (or more). In an embodiment, the linker is (AP)₁₀ and has the amino acid of SEQ ID NO: 37.

In some embodiments, the linker also includes one or more HA tag (SEQ ID NO: 51).

The heterologous enzymes of the present disclosure can be selected or designed to be expressed in a secreted form. In some embodiments, the heterologous enzymes of the present disclosure include a signal peptide sequence (which can be native or heterologous to the heterologous enzyme). It is understood that the signal sequence will be present in the heterologous enzyme when the enzyme is located intracellularly and removed by cleavage when the enzyme is secreted. As used herein, a “signal peptide sequence” refers to a short amino acid sequence presented at the N-terminus of a newly synthesized polypeptide that are destined towards the secretory pathway. Signal sequences can be found on polypeptides that reside either inside certain organelles (the endoplasmic reticulum, golgi or endosomes), secreted from the cell, or inserted into most cellular membranes. In some cases where the heterologous enzyme is secreted from the cell, the signal sequence is cleaved from the heterologous enzyme, freeing the heterologous enzyme for secretion from the cell. In an embodiment, the signal sequence of heterologous enzymes of the present disclosure is endogenous to the heterologous enzyme. In another embodiment, the signal sequence of the heterologous enzymes is heterologous to the heterologous enzyme and can be derived from, for example, a polypeptide known to be secreted from its host. In some embodiments, one or more signal sequences can be used.

In an embodiment of the heterologous enzymes of the present disclosure, the heterologous enzymes include a signal sequence on the N-terminus of the polypeptide. In other embodiments, the heterologous enzymes of the present disclosure lack a signal sequence. In yet other embodiments, the heterologous enzymes of the present disclosure are derived from cleaving the signal sequences of polypeptides having a signal sequence.

In an embodiment, the nucleic acid molecule encoding the heterologous enzyme can include a signal sequence which is endogenous to the host cell expressing the nucleotide molecule. For example, when the host is S. cerevisiae, the nucleic acid molecule encoding the heterologous enzyme can include the signal sequence of a gene endogenously expressed in S. cerevisiae, such as the signal sequence of the invertase gene (SUC2).

In some embodiments, the signal sequence is from the gene encoding the invertase protein (and can have, for example, the amino acid sequence of SEQ ID NO: 38, a variant thereof or a fragment thereof), the AGA2 protein (and can have, for example, the amino acid sequence of SEQ ID NO: 39, a variant thereof or a fragment thereof) or the fungal amylase (and can have, for example, the amino acid sequence of SEQ ID NO: 59, a variant thereof or a fragment thereof). In the context of the present disclosure, the expression “functional variant of a signal sequence” refers to a nucleic acid sequence that has been substituted in at least one nucleic acid position when compared to the native signal sequence which retain the ability to direct the expression of the heterologous enzyme outside the cell. In the context of the present disclosure, the expression “functional fragment of a signal sequence” refers to a shorter nucleic acid sequence than the native signal sequence which retain the ability to direct the expression of the heterologous enzyme outside the cell.

In some embodiments, the heterologous nucleic acid molecule encoding the heterologous enzyme includes a coding sequence for one or a combination of signal sequence(s) allowing the export of the heterologous enzyme outside the yeast host cell's wall. The signal sequence can simply be added to the nucleic acid molecule (usually in frame with the sequence encoding the heterologous enzyme) or replace the signal sequence already present in the heterologous enzyme. The signal sequence can be native or heterologous to the nucleic acid sequence encoding the heterologous enzyme or its corresponding chimera. In some embodiments, one or more signal sequences can be used.

In some embodiments, the heterologous enzyme is a tethered alpha-amylase and have, for example, the amino acid sequence of SEQ ID NO: 65 or 66, a variant thereof or a fragment thereof.

### Tools for Making the Recombinant Yeast Host Cell

In order to make the recombinant yeast host cells, heterologous nucleic acid molecules (also referred to as expression cassettes) are made in vitro and introduced into the yeast host cell in order to allow the recombinant expression of the heterologous enzyme.

The heterologous nucleic acid molecules of the present disclosure comprise a coding region for the heterologous enzyme or a chimeric enzyme comprising the same. A DNA or RNA “coding region” is a DNA or RNA molecule (preferably a DNA molecule) which is transcribed and/or translated into an heterologous enzyme in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

In some embodiments, the heterologous nucleic acid molecules of the present disclosure include a promoter as well as a coding sequence for an heterologous enzyme (including chimeric proteins comprising same). The heterologous nucleic acid sequence can also include a terminator. In the heterologous nucleic acid molecules of the present disclosure, the promoter and the terminator (when present) are operatively linked to the nucleic acid coding sequence of the heterologous enzyme (including chimeric proteins comprising same), e.g., they control the expression and the termination of expression of the nucleic acid sequence of the heterologous enzyme (including chimeric proteins comprising same). The heterologous nucleic acid molecules of the present disclosure can also include a nucleic acid coding for a signal peptide, e.g., a short peptide sequence for exporting the heterologous enzyme outside the host cell. When present, the nucleic acid sequence coding for the signal peptide is directly located upstream and is in frame with the nucleic acid sequence coding for the heterologous enzyme (including chimeric proteins comprising same).

In the heterologous nucleic acid molecule described herein, the promoter and the nucleic acid molecule coding for the heterologous enzyme (including chimeric proteins comprising same) are operatively linked to one another. In the context of the present disclosure, the expressions “operatively linked” or “operatively associated” refers to fact that the promoter is physically associated to the nucleotide acid molecule coding for the heterologous enzyme in a manner that allows, under certain conditions, for expression of the heterologous enzyme from the nucleic acid molecule. In an embodiment, the promoter can be located upstream (5′) of the nucleic acid sequence coding for the heterologous enzyme. In still another embodiment, the promoter can be located downstream (3′) of the nucleic acid sequence coding for the heterologous enzyme. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the heterologous protein. The promoters can be located, in view of the nucleic acid molecule coding for the heterologous enzyme, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. Promoters which cause a gene to be expressed during the propagation phase of a yeast cell are herein referred to as “propagation promoters”. Propagation promoters include both constitutive and inducible promoters, such as, for example, glucose-regulated, molasses-regulated, stress-response promoters (including osmotic stress response promoters) and aerobic-regulated promoters. In a preferred embodiment, the selected promoter allows for the expression of the heterologous nucleic acid molecule during the propagation phase of the recombinant yeast host cell in order to allow a sufficient amount of heterologous enzyme to be expressed. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of the polymerase.

The promoter can be native or heterologous to the nucleic acid molecule encoding the heterologous enzyme. The promoter can be heterologous or derived from a strain being from the same genus or species as the recombinant host cell. In an embodiment, the promoter is derived from the same genus or species of the yeast host cell and the heterologous enzyme is derived from a different genus than the host cell. The promoter can be a single promoter or a combination of different promoters.

In the present disclosure, promoters allowing or favoring the expression of the heterologous enzymes during the propagation phase of the recombinant yeast host cells are preferred. Yeasts that are facultative anaerobes, are capable of respiratory reproduction under aerobic conditions and fermentative reproduction under anaerobic conditions. In many commercial applications, yeast are propagated under aerobic conditions to maximize the conversion of a substrate to biomass. Optionally, the biomass can be used in a subsequent fermentation under anaerobic conditions to produce a desired metabolite. In the context of the present disclosure, it is important that the promoter or combination of promoters present in the heterologous nucleic acid is/are capable of allowing the expression of the heterologous enzyme or its corresponding chimera during the propagation phase of the recombinant yeast host cell. This will allow the accumulation of the heterologous enzyme associated with the recombinant yeast host cell prior to fermentation (if any). In some embodiments, the promoter allows the expression of the heterologous enzyme or its corresponding chimera during propagation, but not during fermentation (if any) of the recombinant yeast host cell.

The promoters can be native or heterologous to the heterologous gene encoding the heterologous enzyme. The promoters that can be included in the heterologous nucleic acid molecule can be constitutive or inducible promoters (such as those described in Perez-Torrado et al., 2005). Inducible promoters include, but are not limited to glucose-regulated promoters (e.g., the promoter of the hxt7 gene (referred to as hxt7p) and having the nucleic acid sequence of SEQ ID NO: 40, a functional variant or a functional fragment thereof; the promoter of the ctt1 gene (referred to as ctt1p) and having the nucleic acid sequence of SEQ ID NO: 41, a functional variant or a functional fragment thereof; the promoter of the glo1 gene (referred to as glo1p) and having the nucleic acid sequence of SEQ ID NO: 42, a functional variant or a functional fragment thereof; the promoter of the ygp1 gene (referred to as ygp1p) and having the nucleic acid sequence of SEQ ID NO: 43, a functional variant or a functional fragment thereof; the promoter of the gsy2 gene (referred to as gsy2p) and having the nucleic acid sequence of SEQ ID NO: 44, a functional variant or a functional fragment thereof), molasses-regulated promoters (e.g., the promoter of the mol1 gene (referred to as mol1p) described in Praekelt et al., 1992 or having the nucleic acid sequence of SEQ ID NO: 45, a functional variant or a functional fragment thereof), heat shock-regulated promoters (e.g., the promoter of the glo1 gene (referred to as glo1p) and having the nucleic acid sequence of SEQ ID NO: 42, a functional variant or a functional fragment thereof; the promoter of the sti1 gene (referred to as sti1p) and having the nucleic acid sequence of SEQ ID NO: 46, a functional variant or a functional fragment thereof; the promoter of the ygp1 gene (referred to as ygp1p) and having the nucleic acid sequence of SEQ ID NO: 43, a functional variant or a functional fragment thereof; the promoter of the gsy2 gene (referred to as gsy2p) and having the nucleic acid sequence of SEQ ID NO: 44, a functional variant or a functional fragment thereof), oxidative stress response promoters (e.g., the promoter of the cup1 gene (referred to as cup1p) and having the nucleic acid sequence of SEQ ID NO: 51, a functional variant or a functional fragment thereof; the promoter of the ctt1 gene (referred to as ctt1p) and having the nucleic acid sequence of SEQ ID NO: 42, a functional variant or a functional fragment thereof; the promoter of the trx2 gene (referred to as trx2p) and having the nucleic acid sequence of SEQ ID NO: 52, a functional variant or a functional fragment thereof; the promoter of the gpd1 gene (referred to as gpd1p) and having the nucleic acid sequence of SEQ ID NO: 53, a functional variant or a functional fragment thereof; the promoter of the hsp12 gene (referred to as hsp12p) and having the nucleic acid sequence of SEQ ID NO: 54, a functional variant or a functional fragment thereof), osmotic stress response promoters (e.g., the promoter of the ctt1 gene (referred to as ctt1p) and having the nucleic acid sequence of SEQ ID NO: 42, a functional variant or a functional fragment thereof; the promoter of the glo1 gene (referred to as glo1p) and having the nucleic acid sequence of SEQ ID NO: 43, a functional variant or a functional fragment thereof; the promoter of the gpd1 gene (referred to as gpd1p) and having the nucleic acid sequence of SEQ ID NO: 53, a functional variant or a functional fragment thereof; the promoter of the ygp1 gene (referred to as ygp1p) and having the nucleic acid sequence of SEQ ID NO: 43, a functional variant or a functional fragment thereof) and nitrogen-regulated promoters (e.g., the promoter of the ygp1 gene (referred to as ygp1p) and having the nucleic acid sequence of SEQ ID NO: 43, a functional variant or a functional fragment thereof).

Promoters that can be included in the heterologous nucleic acid molecule of the present disclosure include, without limitation, the promoter of the tdh1 gene (referred to as tdh1p, a functional variant or a functional fragment thereof), of the hor7 gene (referred to as hor7p, a functional variant or a functional fragment thereof), of the hsp150 gene (referred to as hsp150p, a functional variant or a functional fragment thereof), of the hxt7 gene (referred to as hxt7p, a functional variant or a functional fragment thereof), of the gpm1 gene (referred to as gpm1p, a functional variant or a functional fragment thereof), of the pgk1 gene (referred to as pgk1p, a functional variant or a functional fragment thereof) and/or of the stl1 gene (referred to as stl1p, a functional variant or a functional fragment thereof).

One or more promoters can be used to allow the expression of each heterologous enzyme in the recombinant yeast host cell. In the context of the present disclosure, the expression “functional fragment of a promoter” when used in combination to a promoter refers to a shorter nucleic acid sequence than the native promoter which retain the ability to control the expression of the nucleic acid sequence encoding the heterologous food and/or feed enzyme or its chimera during the propagation phase of the recombinant yeast host cells. Usually, functional fragments are either 5′ and/or 3′ truncation of one or more nucleic acid residue from the native promoter nucleic acid sequence.

In some embodiments, the heterologous nucleic acid molecules include a one or a combination of terminator sequence(s) to end the translation of the heterologous enzyme (or of the chimeric enzyme comprising same). The terminator can be native or heterologous to the nucleic acid sequence encoding the heterologous enzyme or its corresponding chimera. In some embodiments, one or more terminators can be used. In some embodiments, the terminator comprises the terminator from is from the dit1 gene (referred to as dit1, a functional variant or a functional fragment thereof), from the idp1 gene (referred to as idp1t, a functional variant or a functional fragment thereof), from the gpm1 gene (referred to as gpm1t, a functional variant or a functional fragment thereof), from the pma1 gene (referred to as pma1t, a functional variant or a functional fragment thereof), from the tdh3 gene (referred to as tdh3t, a functional variant or a functional fragment thereof), from the hxt2 gene (referred to as hxt2t, a functional variant or a functional fragment thereof), from the adh3 gene (referred to as adh3t, a functional variant or a functional fragment thereof) and/or from the ira2 gene (referred to as ira2t, a functional variant or a functional fragment thereof). In an embodiment, the terminator is derived from the dit1 gene. In another embodiment, the terminator comprises or is derived from the adh3 gene. In the context of the present disclosure, the expression “functional variant of a terminator” refers to a nucleic acid sequence that has been substituted in at least one nucleic acid position when compared to the native terminator which retain the ability to end the expression of the nucleic acid sequence coding for the heterologous protein or its corresponding chimera. In the context of the present disclosure, the expression “functional fragment of a terminator” refers to a shorter nucleic acid sequence than the native terminator which retain the ability to end the expression of the nucleic acid sequence coding for the heterologous enzyme or its corresponding chimera.

In some embodiments, the heterologous nucleic acid molecules include a coding sequence for one or a combination of signal sequence(s) allowing the export of the heterologous enzyme (or of the chimeric enzyme comprising same) outside the yeast host cell's wall. The signal peptide sequence can simply be added to the nucleic acid molecule (usually in frame with the sequence encoding the heterologous enzyme) or replace the signal sequence already present in the heterologous enzyme. The signal sequence can be native or heterologous to the nucleic acid sequence encoding the heterologous enzyme or its corresponding chimera. In some embodiments, one or more signal sequences can be used. In some embodiments, the signal sequence is from the gene encoding the invertase protein (and can have, for example, the amino acid sequence of SEQ ID NO: 39, a variant thereof or a fragment thereof), the AGA2 protein (and can have, for example, the amino acid sequence of SEQ ID NO: 40, a variant thereof or a fragment thereof) or the fungal amylase protein (and can have, for example, the amino acid sequence of SEQ ID NO: 59, a variant thereof or a fragment thereof). In the context of the present disclosure, the expression “functional variant of a signal sequence” refers to a nucleic acid sequence that has been substituted in at least one nucleic acid position when compared to the native signal sequence which retain the ability to direct the expression of the heterologous enzyme or its corresponding chimera outside the cell. In the context of the present disclosure, the expression “functional fragment of a signal sequence” refers to a shorter nucleic acid sequence than the native signal sequence which retain the ability to direct the expression of the heterologous enzyme or its corresponding chimera outside the cell.

The heterologous nucleic acid molecule encoding the heterologous enzyme variant or fragment thereof can be integrated in the genome of the yeast host cell. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the yeast's genome. In such embodiment, the nucleic acid molecule can be stable and self-replicating.

The present disclosure also provides nucleic acid molecules for modifying the yeast host cell so as to allow the expression of the heterologous enzymes, chimeras, variants or fragments thereof. The nucleic acid molecule may be DNA (such as complementary DNA, synthetic DNA or genomic DNA) or RNA (which includes synthetic RNA) and can be provided in a single stranded (in either the sense or the antisense strand) or a double stranded form. The contemplated nucleic acid molecules can include alterations in the coding regions, non-coding regions, or both. Examples are nucleic acid molecule variants containing alterations which produce silent substitutions, additions, or deletions, but do not alter the properties or activities of the encoded enzymes, chimeras, variants or fragments.

In some embodiments, the heterologous nucleic acid molecules which can be introduced into the recombinant host cells are codon-optimized with respect to the intended recipient recombinant yeast host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules can be introduced in the yeast host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The present disclosure also provides nucleic acid molecules that are hybridizable to the complement nucleic acid molecules encoding the heterologous enzymes as well as variants or fragments. A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified, e.g., in Sambrook, J., Fritsch, E. F. and Maniatis, T. MOLECULAR CLONING: A LABORATORY MANUAL, Second Edition, Cold Spring Harbor Laboratory Press, Cold Spring Harbor (1989), particularly Chapter 11 and Table 11.1 therein. The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. For more stringent conditions, washes are performed at higher temperatures in which the washes are identical to those above except for the temperature of the final two 30 min washes in 0.2×SSC, 0.5% SDS are increased to 60° C. Another set of highly stringent conditions uses two final washes in 0.1×SSC, 0.1° A SDS at 65° C. An additional set of highly stringent conditions are defined by hybridization at 0.1×SSC, 0.1% SDS, 65° C. and washed with 2×SSC, 0.1% SDS followed by 0.1×SSC, 0.1% SDS.

Hybridization requires that the two nucleic acid molecules contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived. For hybridizations with shorter nucleic acids, i.e., oligonucleotides, the position of mismatches becomes more important, and the length of the oligonucleotide determines its specificity. In one embodiment the length for a hybridizable nucleic acid is at least about 10 nucleotides. Preferably a minimum length for a hybridizable nucleic acid is at least about 15 nucleotides; more preferably at least about 20 nucleotides; and most preferably the length is at least 30 nucleotides. Furthermore, the skilled artisan will recognize that the temperature and wash solution salt concentration may be adjusted as necessary according to factors such as length of the probe.

### Fermenting Yeast Cell for Making a Fermentation Product

In the context of the present disclosure, the fermenting yeast cell is a yeast cell that can produce a fermentation product under fermentation conditions. Suitable fermenting yeast cells that can be used in the context of the present disclosure can be, for example, from the genus Saccharomyces, Kluyveromyces, Arxula, Debaryomyces, Candida, Pichia, Phaffia, Schizosaccharomyces, Hansenula, Kloeckera, Schwanniomyces, Torula or Yarrowia. Suitable yeast species can include, for example, S. cerevisiae, S. bulderi, S. barnetti, S. exiguus, S. uvarum, S. diastaticus, S. boulardii, C. utilis, K. lactis, K. marxianus or K. fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some further embodiments, the yeast is of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe or Schwanniomyces occidentalis. In one particular embodiment, the yeast is Saccharomyces cerevisiae. In some embodiments, the fermenting yeast cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiment, the fermenting yeast cell can be an oleaginous microalgae host cell (e.g., for example, from the genus Thraustochytrium or Schizochytrium). The yeast cell and the recombinant yeast host cell can be from the same or different genus or species. In an embodiment, the fermenting yeast host cell is from the genus Saccharomyces and, in some embodiments, from the species Saccharomyces cerevisiae. In an embodiment, the yeast cell and the recombinant yeast host cell are from the genus Saccharomyces and, in some embodiments, from the species Saccharomyces cerevisiae.

In some embodiments, the fermenting yeast cell is a recombinant host cell including one or more genetic modifications encoding one or more heterologous proteins.

In some embodiments, the fermenting yeast cell comprises a genetic modification (e.g., a heterologous nucleic acid molecule) for reducing the production of one or more native enzymes that function to produce glycerol or regulate glycerol synthesis, for allowing the production of a polypeptide having glucoamylase activity and/or for reducing the production of one or more native enzymes that function to catabolize formate. Alternatively, the fermenting yeast cell having one of the above genetic modifications is used in combination with one or more recombinant host cells, each having one of the other genetic modifications for reducing the production of one or more native enzymes that function to produce glycerol or regulate glycerol synthesis, for allowing the production of the second polypeptide having glucoamylase activity and/or for reducing the production of one or more native enzymes that function to catabolize formate.

As used in the context of the present disclosure, the expression “reducing the production of one or more native enzymes that function to produce glycerol or regulate glycerol synthesis” refers to a genetic modification which limits or impedes the expression of genes associated with one or more native polypeptides (in some embodiments enzymes) that function to produce glycerol or regulate glycerol synthesis, when compared to a corresponding host strain which does not bear the genetic modification. In some instances, the genetic modification reduces but still allows the production of one or more native polypeptides that function to produce glycerol or regulate glycerol synthesis. In other instances, the genetic modification inhibits the production of one or more native enzymes that function to produce glycerol or regulate glycerol synthesis. In some embodiments, the recombinant host cells bear a plurality of second genetic modifications, wherein at least one reduces the production of one or more native polypeptides and at least another inhibits the production of one or more native polypeptides.

As used in the context of the present disclosure, the expression “native polypeptides that function to produce glycerol or regulate glycerol synthesis” refers to polypeptides which are endogenously found in the recombinant host cell. Native enzymes that function to produce glycerol include, but are not limited to, the GPD1 and the GPD2 polypeptide (also referred to as GPD1 and GPD2 respectively). Native enzymes that function to regulate glycerol synthesis include, but are not limited to, the FPS1 polypeptide. In an embodiment, the recombinant host cell bears a genetic modification in at least one of the gpd1 gene (encoding the GPD1 polypeptide), the gpd2 gene (encoding the GPD2 polypeptide), the fps1 gene (encoding the FPS1 polypeptide) or orthologs thereof. In another embodiment, the fermenting yeast cell bears a genetic modification in at least two of the gpd1 gene (encoding the GPD1 polypeptide), the gpd2 gene (encoding the GPD2 polypeptide), the fps1 gene (encoding the FPS1 polypeptide) or orthologs thereof. In still another embodiment, the recombinant yeast host cell bears a genetic modification in each of the gpd1 gene (encoding the GPD1 polypeptide), the gpd2 gene (encoding the GPD2 polypeptide) and the fps1 gene (encoding the FPS1 polypeptide) or orthologs thereof. Examples of recombinant yeast host cells bearing such genetic modification(s) leading to the reduction in the production of one or more native enzymes that function to produce glycerol or regulate glycerol synthesis are described in WO 2012/138942. Preferably, the fermenting yeast cell has a genetic modification (such as a genetic deletion or insertion) only in one enzyme that functions to produce glycerol, in the gpd2 gene, which would cause the host cell to have a knocked-out gpd2 gene. In some embodiments, the fermenting yeast cell can have a genetic modification in the gpd1 gene, the gpd2 gene and the fps1 gene resulting is a recombinant host cell being knock-out for the gpd1 gene, the gpd2 gene and the fps1 gene.

As used in the context of the present disclosure, the expression “native polypeptides that function to catabolize formate” refers to polypeptides which are endogenously found in the fermenting yeast cell. Native enzymes that function to catabolize formate include, but are not limited to, the FDH1 and the FDH2 polypeptides (also referred to as FDH1 and FDH2 respectively). In an embodiment, the fermenting yeast cell bears a genetic modification in at least one of the fdh1 gene (encoding the FDH1 polypeptide), the fdh2 gene (encoding the FDH2 polypeptide) or orthologs thereof. In another embodiment, the fermenting yeast cell bears genetic modifications in both the fdh1 gene (encoding the FDH1 polypeptide) and the fdh2 gene (encoding the FDH2 polypeptide) or orthologs thereof. Examples of fermenting yeast cells bearing such genetic modification(s) leading to the reduction in the production of one or more native enzymes that function to catabolize formate are described in WO 2012/138942. Preferably, the fermenting yeast cell has genetic modifications (such as a genetic deletion or insertion) in the fdh1 gene and in the fdh2 gene which would cause the host cell to have knocked-out fdh1 and fdh2 genes.

In an embodiment, the recombinant fermenting yeast host cell includes a genetic modification does achieve higher pyruvate formate lyase activity in the recombinant or the further yeast host cell. This increase in pyruvate formate lyase activity is relative to a corresponding native yeast host cell which does not include the first genetic modification. As used in the context of the present disclosure, the term “pyruvate formate lyase” or “PFL” refers to an enzyme (EC 2.3.1.54) also known as formate C-acetyltransferase, pyruvate formate-lyase, pyruvic formate-lyase and formate acetyltransferase. Pyruvate formate lyases are capable of catalyzing the conversion of coenzyme A (CoA) and pyruvate into acetyl-CoA and formate. In some embodiments, the pyruvate formate lyase activity may be increased by expressing an heterologous pyruvate formate lyase activitating enzyme and/or a pyruvate formate lyase enzymate (such as, for example PFLA and/or PFLB).

In the context of the present disclosure, the genetic modification can include the introduction of an heterologous nucleic acid molecule encoding a pyruvate formate lyase activating enzyme and/or a puryvate formate lyase enzyme, such as PFLA. Embodiments of the pyruvate formate lyase activating enzyme and of PFLA can be derived, without limitation, from the following (the number in brackets correspond to the Gene ID number): Escherichia coli (MG1655945517), Shewanella oneidensis (1706020), Bifidobacterium longum (1022452), Mycobacterium bovis (32287203), Haemophilus parasuis (7277998), Mannheimia haemolytica (15341817), Vibrio vulnificus (33955434), Cronobacter sakazakii (29456271), Vibrio alginolyticus (31649536), Pasteurella multocida (29388611), Aggregatibacter actinomycetemcomitans (31673701), Actinobacillus suis (34291363), Finegoldia magna (34165045), Zymomonas mobilis subsp. mobilis (3073423), Vibrio tubiashii (23444968), Gallibacterium anatis (10563639), Actinobacillus pleuropneumoniae serovar (4849949), Ruminiclostridium thermocellum (35805539), Cylindrospermopsis raciborskii (34474378), Lactococcus garvieae (34204939), Bacillus cytotoxicus (33895780), Providencia stuartii (31518098), Pantoea ananatis (31510290), Teredinibacter turnerae (29648846), Morganella morganii subsp. morganii (14670737), Vibrio anguillarum (77510775106), Dickeya dadantii (39379733484), Xenorhabdus bovienii (8830449), Edwardsiella ictaluri (7959196), Proteus mirabilis (6801040), Rahnella aquatilis (34350771), Bacillus pseudomycoides (34214771), Vibrio alginolyticus (29867350), Vibrio nigripulchritudo (29462895), Vibrio orientalis (25689084), Kosakonia sacchari (23844195), Serratia marcescens subsp. marcescens (23387394), Shewanella baltica (11772864), Vibrio vulnificus (2625152), Streptomyces acidiscabies (33082227), Streptomyces davaonensis (31227069), Streptomyces scabiei (24308152), Volvox carteri f. nagariensis (9616877), Vibrio breoganii (35839746), Vibrio mediterranei (34766273), Fibrobacter succinogenes subsp. succinogenes (34755395), Enterococcus gilvus (34360882), Akkermansia muciniphila (34173806), Enterobacter hormaechei subsp. Steigerwaltii (34153767), Dickeya zeae (33924935), Enterobacter sp. (32442159), Serratia odorifera (31794665), Vibrio crassostreae (31641425), Selenomonas ruminantium subsp. lactilytica (31522409), Fusobacterium necrophorum subsp. funduliforme (31520833), Bacteroides uniformis (31507008), Haemophilus somnus (233631487328), Rodentibacter pneumotropicus (31211548), Pectobacterium carotovorum subsp. carotovorum (29706463), Eikenella corrodens (29689753), Bacillus thuringiensis (29685036), Streptomyces rimosus subsp. Rimosus (29531909), Vibrio fluvialis (29387180), Klebsiella oxytoca (29377541), Parageobacillus thermoglucosidans (29237437), Aeromonas veronii (28678409), Clostridium innocuum (26150741), Neisseria mucosa (25047077), Citrobacter freundii (23337507), Clostridium bolteae (23114831), Vibrio tasmaniensis (7160642), Aeromonas salmonicida subsp. salmonicida (4995006), Escherichia coli O157:H7 str. Sakai (917728), Escherichia coli O83:H1 str. (12877392), Yersinia pestis (11742220), Clostridioides difficile (4915332), Vibrio fischeri (3278678), Vibrio parahaemolyticus (1188496), Vibrio coralliilyticus (29561946), Kosakonia cowanii (35808238), Yersinia ruckeri (29469535), Gardnerella vaginalis (99041930), Listeria fleischmannii subsp. Coloradonensis (34329629), Photobacterium kishitanii (31588205), Aggregatibacter actinomycetemcomitans (29932581), Bacteroides caccae (36116123), Vibrio toranzoniae (34373279), Providencia alcalifaciens (34346411), Edwardsiella anguillarum (33937991), Lonsdalea quercina subsp. Quercina (33074607), Pantoea septica (32455521), Butyrivibrio proteoclasticus (31781353), Photorhabdus temperata subsp. Thracensis (29598129), Dickeya solani (23246485), Aeromonas hydrophila subsp. hydrophila (4489195), Vibrio cholerae O1 biovar El Tor str. (2613623), Serratia rubidaea (32372861), Vibrio bivalvicida (32079218), Serratia liquefaciens (29904481), Gilliamella apicola (29851437), Pluralibacter gergoviae (29488654), Escherichia coli O104:H4 (13701423), Enterobacter aerogenes (10793245), Escherichia coli (7152373), Vibrio campbellii (5555486), Shigella dysenteriae (3795967), Bacillus thuringiensis serovar konkukian (2854507), Salmonella enterica subsp. enterica serovar Typhimurium (1252488), Bacillus anthracis (1087733), Shigella flexneri (1023839), Streptomyces griseoruber (32320335), Ruminococcus gnavus (35895414), Aeromonas fluvialis (35843699), Streptomyces ossamyceticus (35815915), Xenorhabdus doucetiae (34866557), Lactococcus piscium (34864314), Bacillus glycinifermentans (34773640), Photobacterium damselae subsp. Damselae 34509297, Streptomyces venezuelae 34035779, Shewanella algae (34011413), Neisseria sicca (33952518), Chania multitudinisentens (32575347), Kitasatospora purpeofusca (32375714), Serratia fonticola (32345867), Aeromonas enteropelogenes (32325051), Micromonospora aurantiaca (32162988), Moritella viscosa (31933483), Yersinia aldovae (31912331), Leclercia adecarboxylata (31868528), Salinivibrio costicola subsp. costicola (31850688), Aggregatibacter aphrophilus (31611082), Photobacterium leiognathi (31590325), Streptomyces canus (31293262), Pantoea dispersa (29923491), Pantoea rwandensis (29806428), Paenibacillus borealis (29548601), Aliivibrio wodanis (28541257), Streptomyces virginiae (23221817), Escherichia coli (7158493), Mycobacterium tuberculosis (887973), Streptococcus mutans (1028925), Streptococcus cristatus (29901602), Enterococcus hirae (13176624), Bacillus licheniformis (3031413), Chromobacterium violaceum (24949178), Parabacteroides distasonis (5308542), Bacteroides vulgatus (5303840), Faecalibacterium prausnitzii (34753201), Melissococcus plutonius (34410474), Streptococcus gallolyticus subsp. gallolyticus (34397064), Enterococcus malodoratus (34355146), Bacteroides oleiciplenus (32503668), Listeria monocytogenes (985766), Enterococcus faecalis (1200510), Campylobacter jejuni subsp. jejuni (905864), Lactobacillus plantarum (1063963), Yersinia enterocolitica subsp. enterocolitica (4713333), Streptococcus equinus (33961143), Macrococcus canis (35294771), Streptococcus sanguinis (4807186), Lactobacillus salivarius (3978441), Lactococcus lactis subsp. lactis (1115478), Enterococcus faecium (12999835), Clostridium botulinum A (5184387), Clostridium acetobutylicum (1117164), Bacillus thuringiensis serovar konkukian (2857050), Cryobacterium flavum (35899117), Enterovibrio norvegicus (35871749), Bacillus acidiceler (34874556), Prevotella intermedia (34516987), Pseudobutyrivibrio ruminis (34419801), Pseudovibrio ascidiaceicola (34149433), Corynebacterium coyleae (34026109), Lactobacillus curvatus (33994172), Cellulosimicrobium cellulans (33980622), Lactobacillus agilis (33975995), Lactobacillus sakei (33973512), Staphylococcus simulans (32051953), Obesumbacterium proteus (29501324), Salmonella enterica subsp. enterica serovar Typhi (1247402), Streptococcus agalactiae (1014207), Streptococcus agalactiae (1013114), Legionella pneumophila subsp. pneumophila str. Philadelphia (119832735), Pyrococcus furiosus (1468475), Mannheimia haemolytica (15340992), Thalassiosira pseudonana (7444511), Thalassiosira pseudonana (7444510), Streptococcus thermophilus (31940129), Sulfolobus solfataricus (1454925), Streptococcus iniae (35765828), Streptococcus iniae (35764800), Bifidobacterium thermophilum (31839084), Bifidobacterium animalis subsp. lactis (29695452), Streptobacillus moniliformis (29673299), Thermogladius calderae (13013001), Streptococcus oralis subsp. tigurinus (31538096), Lactobacillus ruminis (29802671), Streptococcus parauberis (29752557), Bacteroides ovatus (29454036), Streptococcus gordonii str. Challis substr. CH1 (25052319), Clostridium botulinum B str. Eklund 17B (19963260), Thermococcus litoralis (16548368), Archaeoglobus sulfaticallidus (15392443), Ferroglobus placidus (8778929), Archaeoglobus profundus (8739370), Listeria seeligeri serovar 1/2b (32488230), Bacillus thuringiensis (31632063), Rhodobacter capsulatus (31491679), Clostridium botulinum (29749009), Clostridium perfringens (29571530), Lactococcus garvieae (12478921), Proteus mirabilis (6799920), Lactobacillus animalis (32012274), Vibrio alginolyticus (29869205), Bacteroides thetaiotaomicron (31617701), Bacteroides thetaiotaomicron (31617140), Bacteroides cellulosilyticus (29608790), Bacteroides ovatus (29453452), Bacillus mycoides (29402181), Chlamydomonas reinhardtii (5726206), Fusobacterium periodonticum (35833538), Selenomonas flueggei (32477557), Selenomonas noxia (32475880), Anaerococcus hydrogenalis (32462628), Centipeda periodontii (32173931), Centipeda periodontii (32173899), Streptococcus thermophilus (31938326), Enterococcus durans (31916360), Fusobacterium nucleatum (31730399), Anaerostipes hadrus (31625694), Anaerostipes hadrus (31623667), Enterococcus haemoperoxidus (29838940), Gardnerella vaginalis (29692621), Streptococcus salivarius (29397526), Klebsiella oxytoca (29379245), Bifidobacterium breve (29241363), Actinomyces odontolyticus (25045153), Haemophilus ducreyi (24944624), Archaeoglobus fulgidus (24793671), Streptococcus uberis (24161511), Fusobacterium nucleatum subsp. animalis (23369066), Corynebacterium accolens (23249616), Archaeoglobus veneficus (10394332), Prevotella melaninogenica (9497682), Aeromonas salmonicida subsp. salmonicida (4997325), Pyrobaculum islandicum (4616932), Thermofilum pendens (4600420), Bifidobacterium adolescentis (4556560), Listeria monocytogenes (986485), Bifidobacterium thermophilum (35776852), Methanothermobacter sp. CaT2 (24854111), Streptococcus pyogenes (901706), Exiguobacterium sibiricum (31768748), Clostridioides difficile (4916015), Clostridioides difficile (4913022), Vibrio parahaemolyticus (1192264), Yersinia enterocolitica subsp. enterocolitica (4712948), Enterococcus cecorum (29475065), Bifidobacterium pseudolongum (34879480), Methanothermus fervidus (9962832), Methanothermus fervidus (9962056), Corynebacterium simulans (29536891), Thermoproteus uzoniensis (10359872), Vulcanisaeta distributa (9752274), Streptococcus mitis (8799048), Ferroglobus placidus (8778420), Streptococcus suis (8153745), Clostridium novyi (4541619), Streptococcus mutans (1029528), Thermosynechococcus elongatus (1010568), Chlorobium tepidum (1007539), Fusobacterium nucleatum subsp. nucleatum (993139), Streptococcus pneumoniae (933787), Clostridium baratii (31579258), Enterococcus mundtii (31547246), Prevotella ruminicola (31500814), Aeromonas hydrophila subsp. hydrophila (4490168), Aeromonas hydrophila subsp. hydrophila (4487541), Clostridium acetobutylicum (1117604), Chromobacterium subtsugae (31604683), Gilliamella apicola (29849369), Klebsiella pneumoniae subsp. pneumoniae (11846825), Enterobacter cloacae subsp. cloacae (9125235), Escherichia coli (7150298), Salmonella enterica subsp. enterica serovar Typhimurium (1252363), Salmonella enterica subsp. enterica serovar Typhi (1247322),Bacillus cereus (1202845), Bacteroides thetaiotaomicron (1074343), Bacteroides thetaiotaomicron (1071815), Bacillus coagulans (29814250), Bacteroides cellulosilyticus (29610027), Bacillus anthracis (2850719), Monoraphidium neglectum (25735215), Monoraphidium neglectum (25727595), Alloscardovia omnicolens (35868062), Actinomyces neuii subsp. neuii (35867196), Acetoanaerobium sticklandii (35557713), Exiguobacterium undae (32084128), Paenibacillus pabuli (32034589), Paenibacillus etheri (32019864), Actinomyces oris (31655321), Vibrio alginolyticus (31651465), Brochothrix thermosphacta (29820407), Lactobacillus sakei subsp. sakei (29638315), Anoxybacillus gonensis (29574914), variants thereof as well as fragments thereof. In an embodiment, the PFLA protein is derived from the genus Bifidobacterium and in some embodiments from the species Bifidobacterium adolescentis. In an embodiment, the heterologous nucleic acid molecule encoding the PFLA protein is present in at least one, two, three, four, five or more copies in the recombinant yeast host cell. In still another embodiment, the heterologous nucleic acid molecule encoding the PFLA protein is present in no more than five, four, three, two or one copy/ies in the recombinant yeast host cell.

In the context of the present disclosure, the recombinant fermenting yeast host cell has a genetic modification encoding a formate acetyltransferase enzyme and/or a puryvate formate lyase enzyme, such as PFLB. Embodiments of PFLB can be derived, without limitation, from the following (the number in brackets correspond to the Gene ID number): Escherichia coli (945514), Shewanella oneidensis (1170601), Actinobacillus suis (34292499), Finegoldia magna (34165044), Streptococcus cristatus (29901775), Enterococcus hirae (13176625), Bacillus (3031414), Providencia alcalifaciens (34345353), Lactococcus garvieae (34203444), Butyrivibrio proteoclasticus (31781354), Teredinibacter turnerae (29651613), Chromobacterium violaceum (24945652), Vibrio campbellii (5554880), Vibrio campbellii (5554796), Rahnella aquatilis HX2 (34351700), Serratia rubidaea (32375076), Kosakonia sacchari SP1 (23845740), Shewanella baltica (11772863), Streptomyces acidiscabies (33082309), Streptomyces davaonensis (31227068), Parabacteroides distasonis (5308541), Bacteroides vulgatus (5303841), Fibrobacter succinogenes subsp. succinogenes (34755392), Photobacterium damselae subsp. Damselae (34512678), Enterococcus gilvus (34361749), Enterococcus gilvus (34360863), Enterococcus malodoratus (34355213), Enterococcus malodoratus (34354022), Akkermansia muciniphila (34174913), Lactobacillus curvatus (33995135), Dickeya zeae (33924934), Bacteroides oleiciplenus (32502326), Micromonospora aurantiaca (32162989), Selenomonas ruminantium subsp. lactilytica (31522408), Fusobacterium necrophorum subsp. funduliforme (31520832), Bacteroides uniformis (31507007), Streptomyces rimosus subsp. Rimosus (29531908), Clostridium innocuum (26150740), Haemophilus] ducreyi (24944556), Clostridium bolteae (23114829), Vibrio tasmaniensis (7160644), Aeromonas salmonicida subsp. salmonicida (4997718), Listeria monocytogenes (986171), Enterococcus faecalis (1200511), Lactobacillus plantarum (1064019), Vibrio fischeri (3278780), Lactobacillus sakei (33973511), Gardnerella vaginalis (9904192), Vibrio vulnificus (33954428), Vibrio toranzoniae (34373229), Anaerostipes hadrus (34240161), Edwardsiella anguillarum (33940299), Edwardsiella anguillarum (33937990), Lonsdalea quercina subsp. Quercina (33074710), Enterococcus faecium (12999834), Aeromonas hydrophila subsp. hydrophila (4489100), Clostridium acetobutylicum (1117163), Escherichia coli (7151395), Shigella dysenteriae (3795966), Bacillus thuringiensis serovar konkukian (2856201), Salmonella enterica subsp. enterica serovar Typhimurium (1252491), Shigella flexneri (1023824), Streptomyces griseoruber (32320336), Cryobacterium flavum (35898977), Ruminococcus gnavus (35895748), Bacillus acidiceler (34874555), Lactococcus piscium (34864362), Vibrio mediterranei (34766270), Faecalibacterium prausnitzii (34753200), Prevotella intermedia (34516966), Photobacterium damselae subsp. Damselae (34509286), Pseudobutyrivibrio ruminis (34419894), Melissococcus plutonius (34408953), Streptococcus gallolyticus subsp. gallolyticus (34398704), Enterobacter hormaechei subsp. Steigerwaltii (34155981), Enterobacter hormaechei subsp. Steigerwaltii (34152298), Streptomyces venezuelae (34036549), Shewanella algae (34009243), Lactobacillus agilis (33976013), Streptococcus equinus (33961013), Neisseria sicca (33952517), Kitasatospora purpeofusca (32375782), Paenibacillus borealis (29549449), Vibrio fluvialis (29387150), Aliivibrio wodanis (28542465), Aliivibrio wodanis (28541256), Escherichia coli (7157421), Salmonella enterica subsp. enterica serovar Typhi (1247405), Yersinia pestis (1174224), Yersinia enterocolitica subsp. enterocolitica (4713334), Streptococcus suis (8155093), Escherichia coli (947854), Escherichia coli (946315), Escherichia coli (945513), Escherichia coli (948904), Escherichia coli (917731), Yersinia enterocolitica subsp. enterocolitica (4714349), variants thereof as well as fragments thereof. In an embodiment, the PFLB protein is derived from the genus Bifidobacterium and in some embodiments from the specifies Bifidobacterium adolescentis. In an embodiment, the heterologous nucleic acid molecule encoding the PFLB protein is present in at least one, two, three, four, five or more copies in the recombinant yeast host cell. In still another embodiment, the heterologous nucleic acid molecule encoding the PFLB protein is present in no more than five, four, three, two or one copy/ies in the recombinant yeast host cell.

In some embodiments, the recombinant fermenting yeast host cell comprises a first genetic modification for expressing a PFLA protein, a PFLB protein or a combination. In a specific embodiment, the recombinant fermenting yeast host cell comprises a first genetic modification for expressing a PFLA protein and a PFLB protein which can, in some embodiments, be provided on distinct heterologous nucleic acid molecules. As indicated below, the recombinant fermenting yeast host cell can also include additional genetic modifications to provide or increase its ability to transform acetyl-CoA into an alcohol such as ethanol.

Alternatively or in combination, the recombinant fermenting yeast host cell can bear one or more genetic modification for utilizing acetyl-CoA for example, by providing or increasing acetaldehyde and/or alcohol dehydrogenase activity. Acetyl-coA can be converted to an alcohol such as ethanol using first an acetaldehyde dehydrogenase and then an alcohol dehydrogenase. Acylating acetaldehyde dehydrogenases (E.C. 1.2.1.10) are known to catalyze the conversion of acetaldehyde into acetyl-coA in the presence of coA. Alcohol dehydrogenases (E.C. 1.1.1.1) are known to be able to catalyze the conversion of acetaldehyde into ethanol. The acetaldehyde dehydrogenase and alcohol dehydrogenase activity can be provided by a single protein (e.g., a bifunctional acetaldehyde/alcohol dehydrogenase) or by a combination of more than one protein (e.g., an acetaldehyde dehydrogenase and an alcohol dehydrogenase). In embodiments in which the acetaldehyde/alcohol dehydrogenase activity is provided by more than one protein, it may not be necessary to provide the combination of proteins in a recombinant form in the recombinant yeast host cell as the cell may have some pre-existing acetaldehyde or alcohol dehydrogenase activity. In such embodiments, the genetic modification can include providing one or more heterologous nucleic acid molecule encoding one or more of an heterologous acetaldehyde dehydrogenase (AADH), an heterologous alcohol dehydrogenase (ADH) and/or heterologous bifunctional acetylaldehyde/alcohol dehydrogenases (ADHE). For example, the genetic modification can comprise introducing an heterologous nucleic acid molecule encoding an acetaldehyde dehydrogenase. In another example, the genetic modification can comprise introducing an heterologous nucleic acid molecule encoding an alcohol dehydrogenase. In still another example, the genetic modification can comprise introducing at least two heterologous nucleic acid molecules, a first one encoding an heterologous acetaldehyde dehydrogenase and a second one encoding an heterologous alcohol dehydrogenase. In another embodiment, the genetic modification comprises introducing an heterologous nucleic acid encoding an heterologous bifunctional acetylaldehyde/alcohol dehydrogenases (AADH) such as those described in U.S. Pat. No. 8,956,851 and WO 2015/023989. Heterologous AADHs of the present disclosure include, but are not limited to, the ADHE polypeptides or a polypeptide encoded by an adhe gene ortholog.

The recombinant fermenting yeast host cell can be further genetically modified to allow for the production of additional heterologous polypeptides. In an embodiment, the recombinant fermenting yeast cell can be used for the production of an enzyme, and especially an enzyme involved in the cleavage or hydrolysis of its substrate (e.g., a lytic enzyme and, in some embodiments, a saccharolytic enzyme). In still another embodiment, the enzyme can be a glycoside hydrolase. In the context of the present disclosure, the term “glycoside hydrolase” refers to an enzyme involved in carbohydrate digestion, metabolism and/or hydrolysis, including amylases (other than those described above), cellulases, hemicellulases, cellulolytic and amylolytic accessory enzymes, inulinases, levanases, trehalases, pectinases, and pentose sugar utilizing enzymes. In another embodiment, the enzyme can be a protease. In the context of the present disclosure, the term “protease” refers to an enzyme involved in protein digestion, metabolism and/or hydrolysis. In yet another embodiment, the enzyme can be an esterase. In the context of the present disclosure, the term “esterase” refers to an enzyme involved in the hydrolysis of an ester from an acid or an alcohol, including phosphatases such as phytases.

In order to make the recombinant fermenting yeast host cells, one or more heterologous nucleic acid molecules (also referred to as expression cassettes) may be made in vitro and introduced into the fermenting yeast cell in order to allow the recombinant expression of the polypeptides described herein.

### Yeast Products and Processes for Making Yeast Products

The yeast cells of the present disclosure can be used in the preparation of a yeast product which can ultimately be used as an additive to improve the yield of a fermentation by a fermenting yeast cell. In some embodiments in which the yeast cell is the a recombinant yeast host cell, the yeast products made by the process of the present disclosure can comprise at least 0.1% (in dry weight percentage) of the heterologous enzyme when compared the total proteins of the yeast product. The yeast products of the present disclosure can include one or more heterologous enzymes as described herein. In another embodiment, the present disclosure provides processes as well as yeast products having a specific minimal enzymatic activity and/or a specific range of enzymatic activity. Advantageously, the heterologous enzyme present in some embodiments of the yeast products can be concentrated during processing and can remain biologically active to perform its intended function in the yeast products.

When the yeast product is an inactivated yeast product, the process for making the yeast product broadly comprises two steps: a first step of providing propagated yeast host cells and a second step of lysing the propagated yeast host cells for making the yeast product. The process for making the yeast product can include an optional separating step and an optional drying step. In some embodiments, the process can include providing the propagated yeast host cells which have been propagated on molasses. Alternatively, the process can include providing the propagated yeast host cells are propagated on a medium comprising a yeast extract. In some embodiment, the process can further comprises propagating the yeast host cells (on a molasses or YPD medium for example).

In some embodiments, the cells can be lysed using autolysis (which can be optionally be performed in the presence of additional exogenous enzymes) or homogenized (for example using a bead milling, bead beating or a high pressure homogenizing technique).

In some embodiments, the propagated recombinant yeast host cells can be lysed using autolysis. For example, the propagated recombinant yeast host cells may be subject to a combined heat and pH treatment for a specific amount of time (e.g., 24 h) in order to cause the autolysis of the propagated recombinant yeast host cells to provide the lysed recombinant yeast host cells. For example, the propagated recombinant cells can be submitted to a temperature of between about 40° C. to about 70° C. or between about 50° C. to about 60° C. The propagated recombinant cells can be submitted to a temperature of at least about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C. or 70° C. Alternatively or in combination the propagated recombinant cells can be submitted to a temperature of no more than about 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C., 49° C., 48° C., 47° C., 46° C., 45° C., 44° C., 43° C., 42° C., 41° C. or 40° C. In another example, the propagated recombinant cells can be submitted to a pH between about 4.0 and 8.5 or between about 5.0 and 7.5. The propagated recombinant cells can be submitted to a pH of at least about, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4 or 8.5. Alternatively or in combination, the propagated recombinant cells can be submitted to a pH of no more than 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3., 5.2, 5.1, 5.0, 4.9, 4.8, 4.7, 4.6 or 4.5.

In some embodiments, the yeast host cells can be homogenized (for example using a bead-milling technique, a bead-beating or a high pressure homogenization technique) and as such the process for making the yeast product comprises an homogenizing step.

The process can also include a drying step. The drying step can include, for example, with spray-drying and/or fluid-bed drying. When the yeast product is an autolysate, the process may include directly drying the lysed recombinant yeast host cells after the lysis step without performing an additional separation of the lysed mixture.

To provide additional yeast products, it may be necessary to further separate the components of the lysed recombinant yeast host cells. For example, the cellular wall components (referred to as a “insoluble fraction”) of the lysed recombinant yeast host cell may be separated from the other components (referred to as a “soluble fraction”) of the lysed recombinant yeast host cells. This separating step can be done, for example, by using centrifugation and/or filtration. The process of the present disclosure can include one or more washing step(s) to provide the cell walls or the yeast extract. The yeast extract can be made by drying the soluble fraction obtained.

In an embodiment of the process, the soluble fraction can be further separated prior to drying. For example, the components of the soluble fraction having a molecular weight of more than 10 kDa can be separated out of the soluble fraction. This separation can be achieved, for example, by using filtration (and more specifically ultrafiltration). When filtration is used to separate the components, it is possible to filter out (e.g., remove) the components having a molecular weight less than about 10 kDa and retain the components having a molecular weight of more than about 10 kDa. The components of the soluble fraction having a molecular weight of more than 10 kDa can then optionally be dried to provide a retentate as the yeast product.

When the yeast product is an active/semi-active product, it can be submitting to a concentrating step, e.g. a step of removing part of the propagation medium from the propagated yeast host cells. The concentrating step can include resuspending the concentrated and propagated yeast host cells in the propagation medium (e.g., unwashed preparation) or a fresh medium or water (e.g., washed preparation).

In the process described herein, the yeast product is provided as an inactive form or is created during the liquefaction/fermentation process. The yeast product can be provided in a liquid, semi-liquid or dry form. In some embodiments, the inactivated yeast product is provided in the form of a cream yeast. As used herein, “cream yeast” refers to an active or semi-active yeast product obtained following the propagation of the yeast host cells.

### Process and Kit for Improving the Yield of a Fermentation Product

The present disclosure provides a process for improving the yield of a fermentation product. The process involves liquefying a liquefaction medium into a (liquefied) fermentation medium. Alternatively or in combination, the process involved fermenting the fermentation medium (which may or may not have been liquefied) with a fermenting yeast cell to obtain the fermentation product. The process can be used to improve the yield of ethanol as a fermentation product. The process can also be used to increase the free amino acid and/or the dextrose equivalent in the (liquefied) fermentation medium (compared to the liquefaction medium) so as to increase the yield of the fermentation product.

In order to achieve this yield improvement, the process also comprises including a yeast host cell or a yeast product obtained from the yeast host cell to the liquefaction medium and/or the fermentation medium (which may or maybe have been liquefied).

In an embodiment, a first inactivated yeast product (obtained from a first recombinant yeast host cell comprising a first heterologous nucleic acid encoding a first heterologous enzyme) is added to the liquefaction medium. In such embodiment, the first inactivated yeast product is present during the liquefaction step. It is expected that some components of the first inactivated yeast product will remain in the liquefied medium which can ultimately be used as a fermenting medium.

In another embodiment, a first inactivated yeast product (obtained from a first recombinant yeast host cell comprising a first heterologous nucleic acid encoding a first heterologous enzyme) is added to the fermentation medium. The fermentation medium may have been previously liquefied or not. In such embodiment, the first inactivated yeast product is not added to the liquefaction medium, but is included in the fermentation medium. Alternatively, the first inactivated yeast product can be added to the liquefaction medium and to the fermentation medium (which may or may not have been liquefied).

In another embodiment, an inactivated yeast product is form in situ by including a second recombinant yeast host cell (comprising a second heterologous nucleic acid encoding a second heterologous enzyme) in the liquefaction medium. In such embodiment, the liquefying/heating step will generate a second inactivated yeast product (from the second recombinant yeast host cell) in the liquefied medium which can be used as a fermentation medium. In some embodiments, the second recombinant yeast host cell is not added to the fermentation medium prior to the formation of the second inactivated yeast host cell.

In yet another embodiment, a third inactivated yeast product (obtained from a non-genetically-modified yeast host cell) is added in the liquefaction medium only and is not added directly into the fermentation medium. It is expected that some components of the third inactivated yeast product will remain in the liquefied medium which can ultimately be used as a fermenting medium. In some embodiments, the third inactivated yeast product is added alone or together with additional exogenous enzymes. In one embodiment, the third inactivated yeast product is combined with an exogenous alpha-amylase. In one embodiment, the process includes adding an exogenous alpha-amylase with the third inactivated yeast product to the liquefaction medium.

As used herein, a liquefaction medium comprises relatively intact starch molecules. A liquefied medium is a medium obtained after a liquefaction step (which usually involves a step of heating the liquefaction medium) at least some of the starch molecules have been hydrolyzed. The liquefied medium has a lower viscosity that the liquefaction medium. A fermentation medium is a medium to which a fermenting organism (such as a yeast cell) capable of metabolizing starch to produce a fermentation product (e.g., ethanol and CO₂) has been added. The fermentation medium may have been previously liquefied (e.g., obtained from a liquefied medium). In some embodiments, the fermentation medium was not previously liquefied.

In one embodiment, the process increases the dextrose equivalent of the (liquefied) fermentation medium when compared to the dextrose equivalent of the liquefaction medium. In other embodiments, the process increases the free amino nitrogen of the (liquefied) fermentation medium when compared to the free amino nitrogen of the liquefaction medium. In one embodiment, the process increases both the dextrose equivalent and the free amino nitrogen of the (liquefied) fermentation medium when compared to the liquefaction medium.

The present disclosure also provides a kit for improving the yield of a fermentation product. The kit comprises at least one of: the first inactivated yeast product, the second recombinant yeast host cell, and/or the third inactivated yeast product and at least one component to make the fermentation medium (e.g., a carbohydrate source, a phosphorus source and/or a nitrogen source for example).

The kit can also include instructions on how to use the first inactivated yeast product, the second recombinant yeast host cell and/or the third inactivated yeast product to improve the fermentation yield of the fermenting yeast cell during fermentation. For example, the instructions can indicate when to use, how to use or how much of the first inactivated yeast product, the second recombinant yeast host cell, third inactivated yeast product and/or the fermenting yeast cell. In an embodiment, the kit comprises the dried components to make the fermentation medium. In yet another embodiment, the kit comprises the fermentation medium in a liquid form. In another embodiment, the kit can comprise the fermentation medium in a dried form, which can, in some embodiments, be provided as components to be combined to make the fermentation medium. In still a further embodiment, the fermentation medium of the kit already contains the first and/or the third inactivated yeast product. The components of the kit can be provided in a sterile form.

As used herein, a “medium” is a substrate that is fermentable by the fermenting yeast cell to make at least one fermentation product (such as, for example ethanol). In some embodiments, the medium includes nutrients used by the yeast cell during the fermentation process. Components of the medium may include a carbohydrate source, a phosphorous source and a nitrogen source. The medium can optionally include micronutrients (such as vitamins and minerals), fatty acids, nitrogen, amino acids or a combination thereof. Further, the medium may include components that are not inherently fermentable by the fermenting yeast cell.

In some embodiments, the liquefaction medium, the liquefied fermentation medium and/or the fermentation medium can include or be supplemented with a biomass that can be fermented by the fermenting yeast cell, and includes any type of biomass known in the art and described herein. For example, the biomass can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-protein, extensin, and pro line-rich proteins).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the fermentation products may be used to produce ethanol or higher value added chemicals, such as organic acids, aromatics, esters, acetone and polymer intermediates.

In some embodiments, the fermentation medium may not contain sufficient nutrients necessary for the growth and metabolism of the fermenting yeast cell during fermentation. The first, second and/or third inactivated yeast product of the present disclosure may include nutrients that supplement nutrients natively present in the fermentation medium. The heterologous enzyme present in the first inactivated yeast product and/or the second recombinant yeast host cell may further support the fermentation. For example, where fermentation medium includes starch, the enzyme may be an amylolytic enzyme that breaks down the starch into smaller molecules.

In some embodiments, the first and/or third inactivated yeast product can be formulated to be added to the liquefaction medium at a concentration of at least about 0.00001 g per liter of the liquefaction medium, 0.00005 g per liter of the liquefaction medium, 0.0001 g per liter of the liquefaction medium, 0.0005 g per liter of the liquefaction medium, 0.001 g per liter of the liquefaction medium, 0.005 g per liter of the liquefaction medium, 0.01 g per liter of the liquefaction medium, 0.05 g per liter of the liquefaction medium, 0.1 g per liter of the liquefaction medium, 0.5 g per liter of the liquefaction medium, or even higher. In one embodiment, the first and/or third inactivated yeast product is formulated to be added to the liquefaction medium at a concentration of at least 0.01 g per liter of the liquefaction medium. In one embodiment, the first and/or third inactivated yeast product is formulated to be added to the liquefaction medium at a concentration of at least 0.03 g per liter of the liquefaction medium.

In some embodiments, the second recombinant yeast host cell can be formulated to be added to the liquefaction medium to provide a second inactivated yeast product at a concentration of at least about 0.00001 g per liter of the liquefaction medium, 0.00005 g per liter of the liquefaction medium, 0.0001 g per liter of the liquefaction medium, 0.0005 g per liter of the liquefaction medium, 0.001 g per liter of the liquefaction medium, 0.005 g per liter of the liquefaction medium, 0.01 g per liter of the liquefaction medium, 0.05 g per liter of the liquefaction medium, 0.1 g per liter of the liquefaction medium, 0.5 g per liter of the liquefaction medium, or even higher. In one embodiment, the second recombinant yeast host cell can be formulated to be added to the liquefaction medium to provide a second inactivated yeast product at a concentration of at least 0.01 g per liter of the liquefaction medium. In one embodiment, the second recombinant yeast host cell can be formulated to be added to the liquefaction medium to provide a second inactivated yeast product at a concentration of at least 0.03 g per liter of the liquefaction medium.

In some embodiments, the first inactivated yeast product, is formulated to be added to the fermentation medium at a concentration of at least about 0.00001 g per liter of the fermenting medium, 0.00005 g per liter of the fermentation medium, 0.0001 g per liter of the fermentation medium, 0.0005 g per liter of the fermentation medium, 0.001 g per liter of the fermentation medium, 0.005 g per liter of the fermentation medium 0.01 g per liter of the fermentation medium, 0.05 g per liter of the fermentation medium, 0.1 g per liter of the fermentation medium, 0.5 g per liter of the fermentation medium or even higher. In one embodiment, the process comprises adding the first inactivated yeast product at a concentration of at least 0.01 g per liter of the fermentation medium. In one embodiment, the process comprises adding the first inactivated yeast product at a concentration of at least 0.03 g per liter of the fermentation medium.

In some embodiments, the kit includes the fermenting yeast cell. The inclusion of the fermenting yeast cell allows for combining the elements of the kit to use the process for improving the yield of a fermentation product made by the first yeast cell as described herein.

The inactivated yeast products and recombinant yeast host cells described herein can be used to in a fermentation process to improve/optimize a yield of a fermentation product of the fermented yeast cell. The inactivated yeast products and recombinant yeast host cells are especially useful in combination with a fermentation medium that may not provide sufficient nutrients for the fermenting yeast cell to survive, thrive, reproduce and/or convert biomass into a fermentable product.

The present disclosure provides using the first inactivated yeast product, the second recombinant yeast host cell and/or the third inactivated yeast product with the fermenting yeast cell to provide nutrients to support growth and/or to improve its and, in some embodiments, limiting or avoiding the need of adding additional exogenous source of purified enzymes during fermentation. The use of the inactivated yeast products and/or recombinant yeast host cells may be advantageous because, in some embodiments, it can reduce or eliminate the need to supplement the liquefaction or fermentation medium with external source of purified enzymes (e.g., glucoamylase and/or alpha-amylase) while providing nutrients for the fermenting yeast cell during the fermentation of the fermentation medium into a fermentation product (such as ethanol).

In addition to improving fermentation yields, the use of the inactivated yeast products and/or recombinant yeast host cell may reduce complexity in controlling inputs into the fermentation medium as a single composition is able to provide multiple functionality. Further, costs of supplying the additive(s) may be relatively lower than supplying separate yeast nutrients and enzymes as both are provided from a single recombinant yeast host cell.

In some embodiments in which the heterologous enzyme present in the first and/or second inactivated yeast product is a thermostable alpha-amylase, which can simplify the fermentation process by hydrolyzing starch (including raw starch) mainly during the liquefaction step in a more efficient manner. In some embodiments, the use of a thermostable alpha-amylase as the heterologous enzyme can reduce or wave the use of a further alpha-amylase during the subsequent fermentation step.

In some embodiments, the inactivated yeast products cells can be added to the fermentation medium prior to, at the same time and/or after the fermenting yeast cell is added to the fermentation medium. The inactivated yeast products/recombinant yeast host cells can be added once or multiple times during liquefaction. In an embodiment, the inactivated yeast products are added to the fermentation medium prior to the addition of the fermenting yeast cell. This is especially convenient when the heterologous enzyme is a thermostable alpha-amylase as it will permit heating the starch at high temperatures and liquefying it prior to the addition of the fermenting yeast cell. Alternatively or in combination, the first inactivated yeast product and/or the second recombinant yeast host cell can be used to improve the liquefaction step by increasing the dextrose equivalent or the free amino acid content of the liquefied fermentation medium. In another embodiment, the first inactivated yeast product can be added to the fermentation medium at the same time the fermenting yeast cell. In yet another embodiment, the first inactivated yeast product is added to the fermentation medium after the addition of the fermenting yeast cell. In still another embodiment, the first and/or third inactivated yeast product is added to the fermentation medium prior to and at the same time the fermenting yeast cell is added to the fermentation medium. In yet another embodiment, the first and/or third inactivated yeast product is added to the fermentation medium prior to and after the fermenting yeast cell is added to the fermentation medium. In another embodiment, the first inactivated yeast product is added to the fermentation medium at the same time and after the fermenting yeast cell is added to the fermentation medium. In still yet another embodiment, the first and/or third inactivated yeast product is added to the fermentation medium prior to, at the same time and after the fermenting yeast cell is added to the fermentation medium.

In some embodiments, the first and/or third inactivated yeast product is added to the liquefaction medium such that its concentration is at least 0.00001 g of the additive per L of the liquefaction medium, at least 0.00005 g of the additive per L of the liquefaction medium, at least 0.0001 g of the additive per L of the liquefaction medium, at least 0.0005 g of the additive per L of the liquefaction medium, at least 0.001 g of the additive per L of the liquefaction medium, at least 0.005 g of the additive per L of the liquefaction medium, at least 0.01 g of the additive per L of the liquefaction medium, at least 0.05 g of the additive per L of the liquefaction medium, at least 0.1 g of the additive per L of the liquefaction medium, at least 0.5 g of the additive per L of the liquefaction medium or more. The first and/or third inactivated yeast product can be formulated in a specific dosage form to provide a specific appropriate concentration to the liquefaction medium.

In some embodiments, the second recombinant yeast host cell is added to the liquefaction medium to provide a second inactivated yeast product at concentration is at least 0.00001 g of the additive per L of the liquefaction medium, at least 0.00005 g of the additive per L of the liquefaction medium, at least 0.0001 g of the additive per L of the liquefaction medium, at least 0.0005 g of the additive per L of the liquefaction medium, at least 0.001 g of the additive per L of the liquefaction medium, at least 0.005 g of the additive per L of the liquefaction medium, at least 0.01 g of the additive per L of the liquefaction medium, at least 0.05 g of the additive per L of the liquefaction medium, at least 0.1 g of the additive per L of the liquefaction medium, at least 0.5 g of the additive per L of the liquefaction medium or more. The second recombinant yeast host cell is added to the liquefaction medium to provide a second inactivated yeast product in a specific dosage form to provide a specific appropriate concentration to the liquefaction medium.

In some embodiments, the first inactivated yeast product is added to the fermentation medium such that its concentration is at least 0.00001 g of the additive per L of the fermentation medium, at least 0.00005 g of the additive per L of the fermentation medium, at least 0.0001 g of the additive per L of the fermentation medium, at least 0.0005 g of the additive per L of the fermentation medium, at least 0.001 g of the additive per L of the fermentation medium, at least 0.005 g of the additive per L of the fermentation medium, at least 0.01 g of the additive per L of the fermentation medium, at least 0.05 g of the additive per L of the fermentation medium, at least 0.1 g of the additive per L of the fermentation medium, at least 0.5 g of the additive per L of the fermentation medium or more. The first inactivated yeast product can be formulated in a specific dosage form to provide a specific appropriate concentration to the fermentation medium.

The fermentation process can be performed at temperatures of at least about 25° C., about 28° C., about 30° C., about 31° C., about 32° C., about 33° C., about 34° C., about 35° C., about 36° C., about 37° C., about 38° C., about 39° C., about 40° C., about 41° C., about 42° C., or about 50° C.

In some embodiments, the fermenting step is conducted under anaerobic conditions. As described above, yeast tends to undergo fermentation processes while under anaerobic conditions, while it tends to undergo propagation processes while under aerobic conditions. As used herein, “anaerobic conditions” means that the liquefaction medium is under an oxygen-poor environment. An oxygen-poor environment may have an oxygen concentration below that of air. For example, the concentration of oxygen may be below 21%, 20%, 15%, 10%, 9%, 8%, 7%, 6%, 5%, 4%, 3%, 2% or 1% by volume.

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, or at least about 500 mg per hour per liter.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

The present invention will be more readily understood by referring to the following examples which are given to illustrate the invention rather than to limit its scope.

### EXAMPLE I—CHARACTERIZATION OF A YEAST EXTRACT COMPRISING ALPHA-AMYLASE ON THE GROWTH AND FERMENTATION PERFORMANCES OF YEAST STRAINS

Lab-scale liquefaction. Cells from strain M15958 were propped in YPD overnight, centrifuged, washed, then dosed at 0.9 g dry cell weight into a 300 ml liquefaction at 85° C. Liquefactions were performed using 33% corn flour with 40% backset at pH 5.3. The slurry was raised to 60° C. and 0.9 g/L of strain M15958 added and the temperature raised 2° C./min to 85° C. Samples were run in a Dinitrosalicylic Acid Reagent Solution (DNS) assay using 25 μl of 1:8 diluted sample with 50 μl DNS and boiled for 5 mins. The absorbance was read at 540 nm and the dextrose equivalent (DE) calculated using a dextrose standard curve.

Microtiter plate growth in Verduyn medium. Growth assays were performed using plate readers to monitor optical density at 600 nm as a function of time. Cell were pre-grown in Verduyn medium (Verduyn et al. 1992) using 40 g/L glucose at pH 4.2, then diluted 1:1000 in fresh media supplemented with either 0, 0.05, 0.1, or 0.5 g/L of a commercial yeast extract. Assays were incubated at 32° C. for 30 h.

Lab-scale growth in Verduyn medium. Fermentation experiments were performed using 50 ml of Verduyn medium at pH 4.2 in 250 mL Pyrex® bottles, with either 0, 0.01, 0.1, or 0.5 g/L yeast extract added. Inoculums were grown overnight in Verduyn media, centrifuged and washed before being dosed at 0.1 g/L dry cell weight. The CO₂ off gas was collected using a CO₂ monitor system. The amount of ethanol and glycerol was determined by high-performance liquid chromatography.

TABLE 1
Description of the strains used in this example. All
strains were derived from a wild-type (not genetically
modified) Saccharomyces cerevisiae M2390 strain.

| | | Native genes |
|---|---|---|
| Name | Heterologous protein expressed | inactivated |
| M15958 | A chimeric protein of formula (I): | Δfcy1 |
| | (NH₂) - SS - AA - L - TT (COOH) | |
| | in which SS has the amino acid sequence of | |
| | SEQ ID NO: 39, AA has the amino acid | |
| | sequence of SEQ ID NO: 13, L has the amino | |
| | acid sequence of SEQ ID NO: 32, and TT has | |
| | the amino acid sequence of SEQ ID NO: 18. | |
| | The chimeric protein was engineered at 2 | |
| | copies per chromosome under the control of | |
| | the TEF2p and the IDP1t and ADH1p and | |
| | DIT1T | |
| M8841 | Gene encoding Saccharomycopsis fibuligera | Δgpd2, |
| | glu0111 (GeneBank Accession CAC83969.1) | Δfdh1, |
| | Gene encoding the PFLA polypeptide | Δfdh2, |
| | (UniProtKB Accession A1A239) | Δfcy1 |
| | Gene encoding the PFLB polypeptide | |
| | (UniProtKB Accession A1A240) | |
| | Gene encoding the ADHE polypeptide | |
| | (UniProtKB Accession A1A067) | |
| M11589 | Gene encoding Saccharomycopsis fibuligera | Δgpd2, |
| | glu0111 (GeneBank Accession CAC83969.1) | Δfdh1, |
| | Gene encoding the PFLA polypeptide | Δfdh2, |
| | (UniProtKB Accession A1A239) | Δfcy1 |
| | Gene encoding the PFLB polypeptide | |
| | (UniProtKB Accession A1A240) | |
| | Gene encoding the ADHE polypeptide | |
| | (UniProtKB Accession A1A067) | |
| | Gene encoding Saccharomyces cerevisiae | |
| | STL1 (GeneBank Accession NP_010825) | |

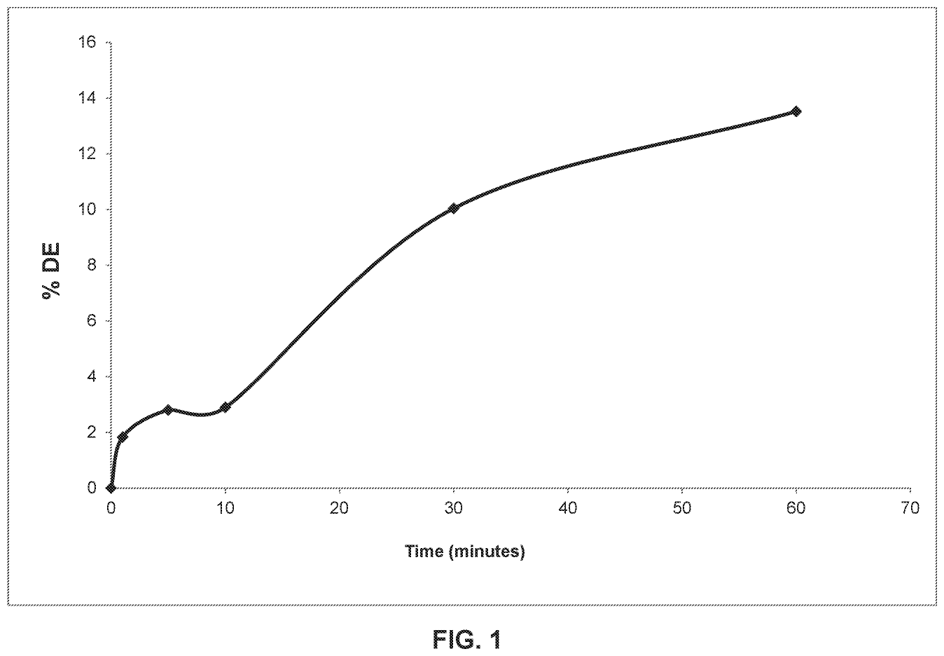

Strain M15958 was grown overnight in YPD₄₀, concentrated into a high cell density slurry with 200 g/L dry cell weight (DCW) and dosed into a lab-scale liquefaction using 0.9 g/L DCW yeast. The yeast product obtained from strain M15958 was able to reach industrially relevant hydrolysis within a 60 min liquefaction without the addition of exogenous enzyme (FIG. 1).

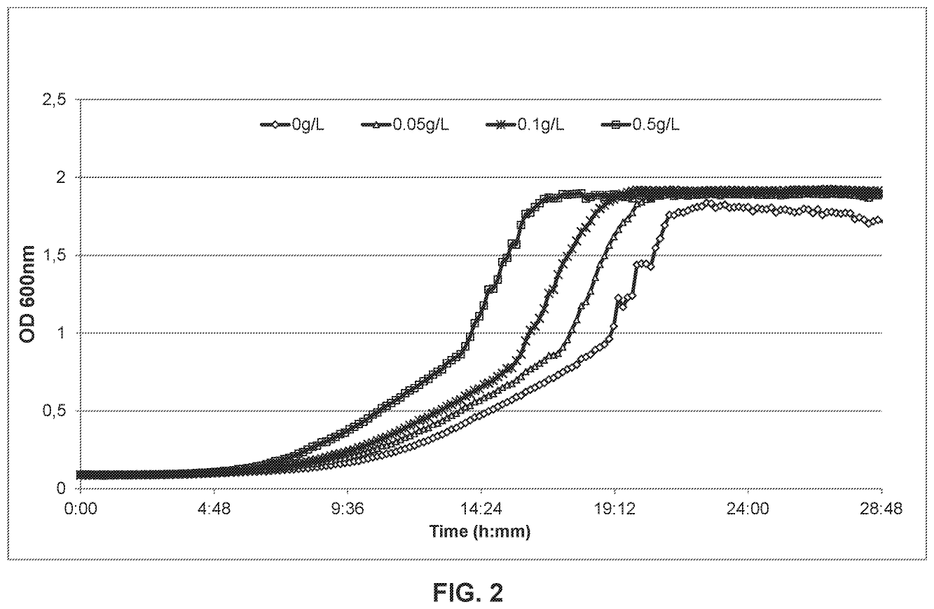

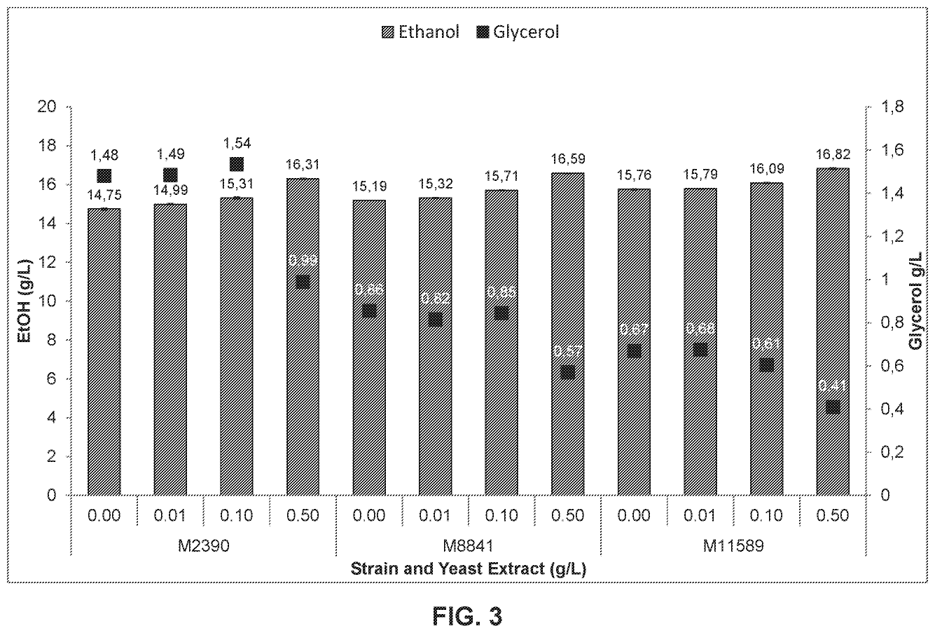

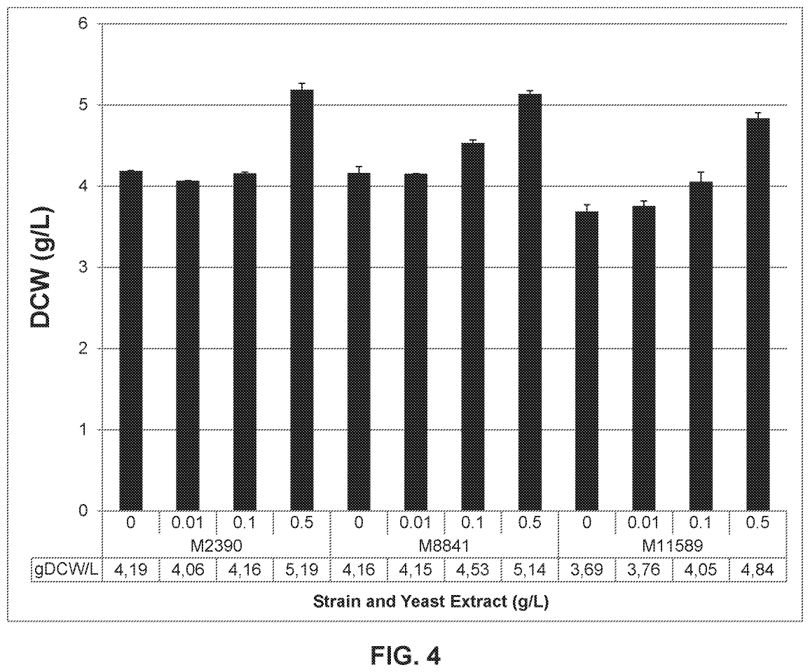

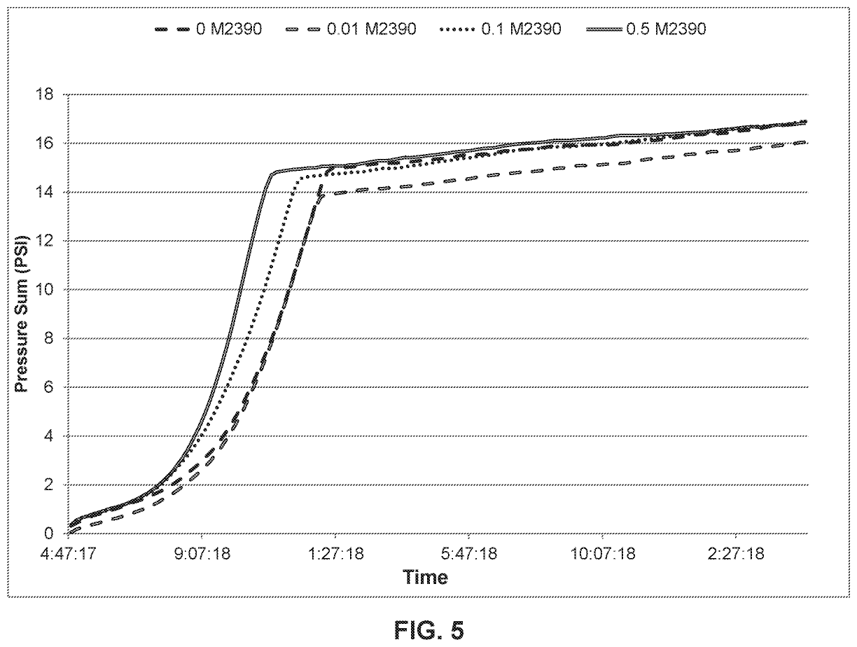

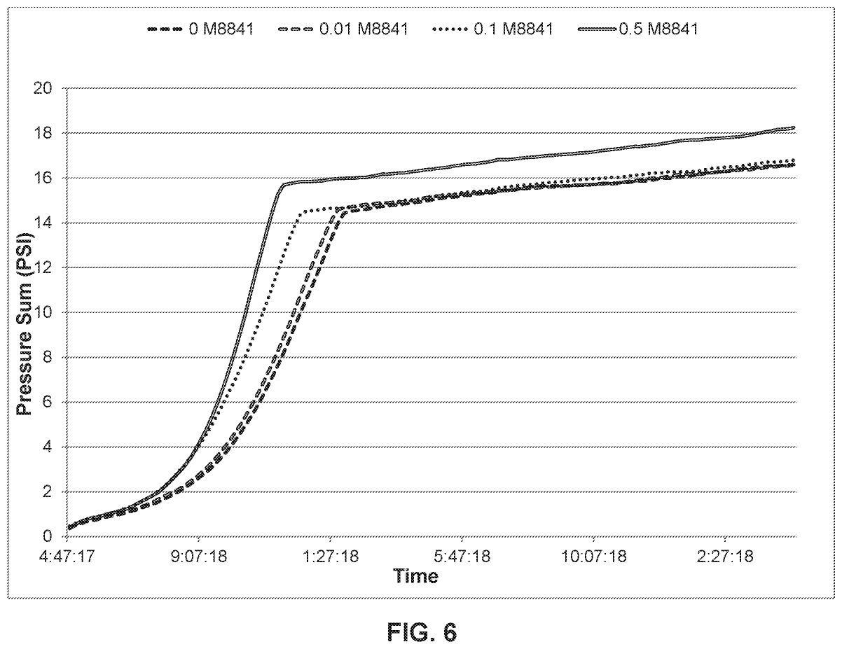

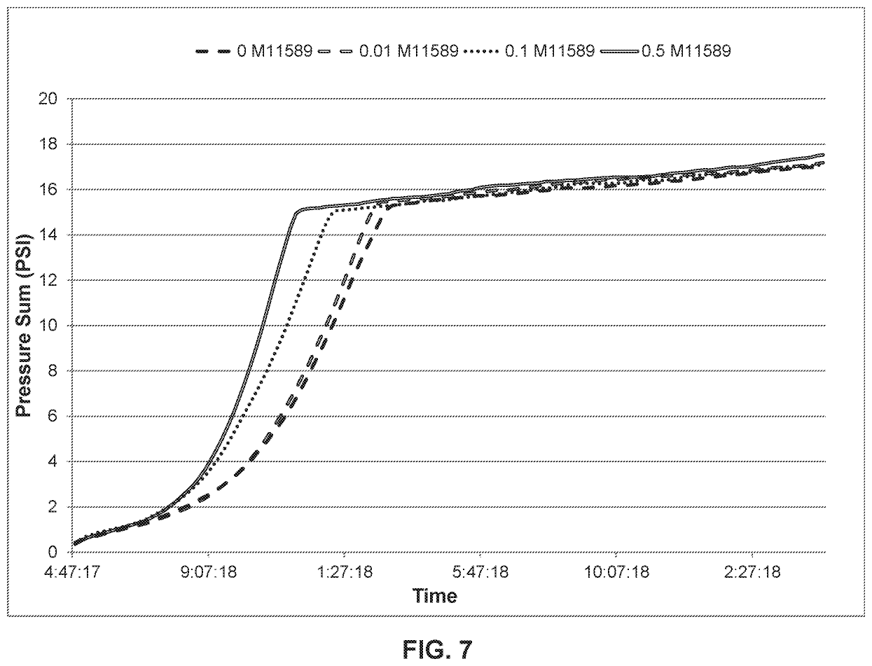

FIG. 2 depicts a microtiter plate reader experiment in which the growth of a glycerol reduction strain, M11589, was improved with the titrated additions of yeast extract as the lag phase is significantly reduced. Similarly, the addition of yeast extract in an anaerobic fermentation on defined Verduyn media showed an improvement in ethanol production and glycerol reduction for a conventional strain, M2390, and two separate glycerol reduction strains, M8841 and M11589 (FIG. 3). All three strains showed an improvement in biomass production (FIG. 4) along with improved growth kinetics as measured by CO₂ production (FIGS. 5 to 7).

### EXAMPLE II—YIELD IMPROVEMENTS IN FERMENTATION USING LIQUEFACTIONS CONTAINING YEAST EXTRACT

Lab-scale liquefaction: Cells from the wild type strain (non-genetically modified) M10474, were propped in YPD overnight, centrifuged, washed, and bead beaten using 0.2 μm glass beads in an MP Biomedical benchtop homogenizer for 3 min. Bead beaten cells were dosed at either 0.012%, 0.03%, or 0.3% grams of dry cell weight per grams of corn solids, into a 300 mL liquefaction, along with a water addition control, all using 0.02% grams of commercial thermostable alpha-amylase per grams of solids. Liquefactions were performed using 34% corn flour with 40% thin stillage at pH 5.2. The slurry was raised to 70° C. before the enzyme and yeast addition, and the temperature raised 2° C./min to 85° C. where it was held for 2 h. After liquefaction, the samples were cooled to room temperature and the solids and pH adjusted to 33% and 4.8 for a subsequent fermentation.

Lab scale fermentations: Fermentations were performed using 50 g of the adjusted 33% solids lab-scale liquefaction in a 200 mL bottle, in duplicates. Each fermentation received the same doses of 500 ppm urea, 0.6 AGU/gram total solids commercial glucoamylase, and 0.05 g/L inoculum of the wild-type (non-genetically modified) M2390 strain. The fermentations were mixed at 150 rpm and incubated at 33° C. for 24 h and the temperatures dropped to 31° C. for the remainder of the fermentation. Samples were collected after 54 h and the ethanol and glucose quantified using high performance liquid chromatography (HPLC).

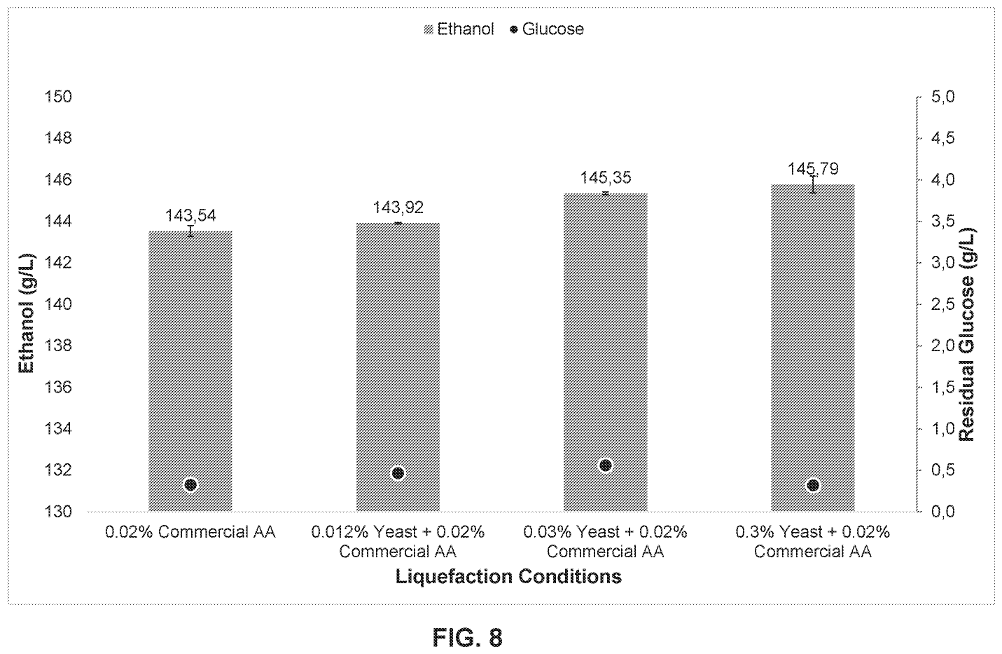

With as little as 0.012% w/w of the M10474 yeast added to the liquefaction, there was an observed 0.26% ethanol yield increase in the subsequent fermentation (FIG. 8). A 1.26% yield increase was observed with the addition of 0.03% yeast, and an additional modest increase at 1.57% ethanol with 0.3% yeast The data presented in FIG. 8 showed that nutrient and yield benefits can be added using disrupted cell cultures in the liquefaction rather than in fermentation.

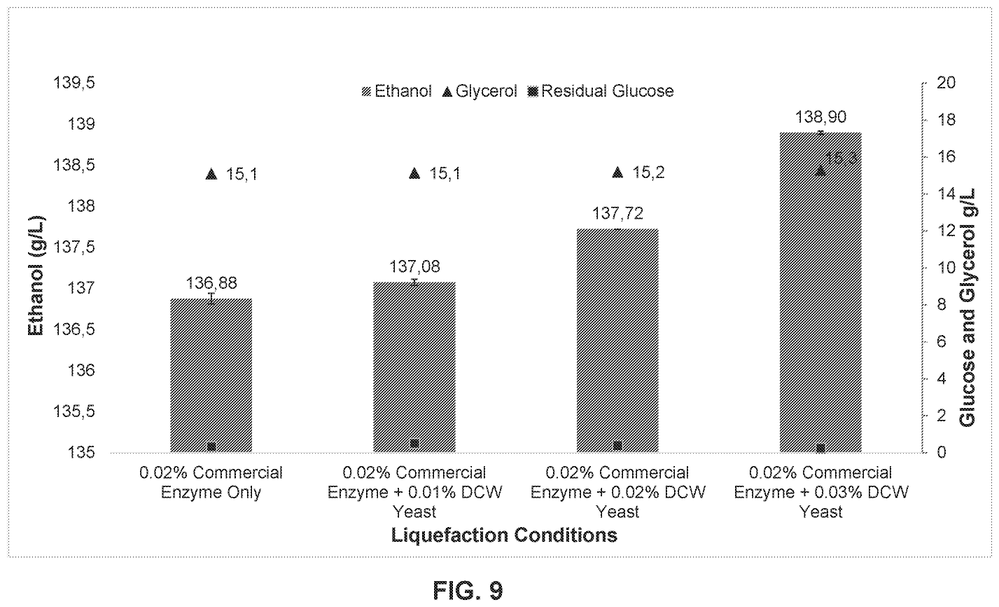

A subsequent liquefaction and fermentation were performed using the same aforementioned lab-scale liquefaction and fermentation protocols, except using 33% solids for the liquefaction and 32% solids for the fermentation. In this experiment, the bead beaten doses of wild type strain M10474 were added at either 0.01%, 0.02%, or 0.03% grams of dry cell weight per grams of corn solids, along with each liquefaction dosed at 0.02% commercial alpha-amylase enzyme. The fermentations were analyzed using HPLC to quantify ethanol, glycerol, and residual glucose. As seen in FIG. 9, the yeast added liquefactions at either 0.01%, 0.02%, or 0.03% DCW of M10474 provided a 0.15%, 0.61%, and a 1.48% yield increase compared to the commercial enzyme only condition.

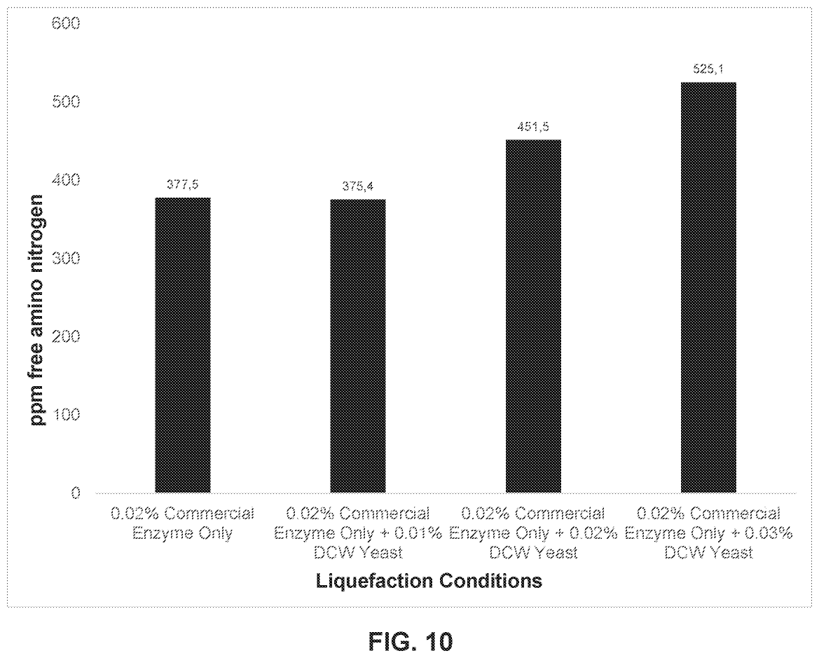

The liquefactions were also analyzed for free amino nitrogen (FAN) using a plate based assay as described in Abernathy et al., 2009. The liquefactions were normalized to 32% solids and compared to a FAN standard curve to estimate the concentrations in parts per million. As seen in FIG. 10, the 0.02% and 0.03% yeast additions provided a 20% and 39% increase in FAN compared to the commercial enzyme only control, whereas there was no measurable change in the 0.01% yeast condition.

### EXAMPLE III—YIELD IMPROVEMENTS IN FERMENTATION USING LIQUEFACTIONS CONTAINING YEAST EXTRACT DERIVED FROM THERMOSTABLE ALPHA-AMYLASE EXPRESSING YEAST STRAINS

Strain M19211 was engineered co-expressing the tethered thermostable alpha-amylase from both P. furiosus and T. hydrothermalis. The M19211 was constructed using a M16449 background expressing a 2 copy per chromosome tethered P. furiosus cassette designed to express the tethered P. furiosus thermostable alpha-amylase (having SEQ ID NO: 65), and 4 copy per chromosome T. hydrothermalis cassette designed to express the tethered T. hydrothermalis thermostable alpha-amylase (having SEQ ID NO: 66) (see Table 2).

The M19211 strain was prepared by either YPD propping overnight, or via a cream yeast production using molasses. The cream yeast was either washed with water and resuspended to approximately 20% total DCW with water, or not washed and resuspended to 20% solids in spent beer. The cream samples were disrupted using a high pressure homogenizer between 1000 and 1500 bar. The YPD propped culture was concentrated in spent supernatant and bead beaten for 3 min using the benchtop homogenizer. The disrupted cultures were each dosed at 0.03% grams DCW per grams of corn solids along with a 25% dose of commercial alpha-amylase enzyme (0.005% weight of enzyme per weight corn solids).

Liquefactions were performed using 34% corn flour with 40% backset at pH 5.2 at 300 mL volumes. The slurry was raised to 70° C. followed by enzyme and yeast additions, and the temperature raised 2° C./min to 85° C. The changes in viscosity were measured using the IKA Microstar30 and Labworldsoft software. Samples were taken after 2 h and mixed with 1% sulfuric acid to stop hydrolysis. Each samples was measured for reducing sugars using the DNS assay and correlated to a dextrose standard curve to correlate dextrose concentrations and expressed as a percentage on a total solids dry basis.

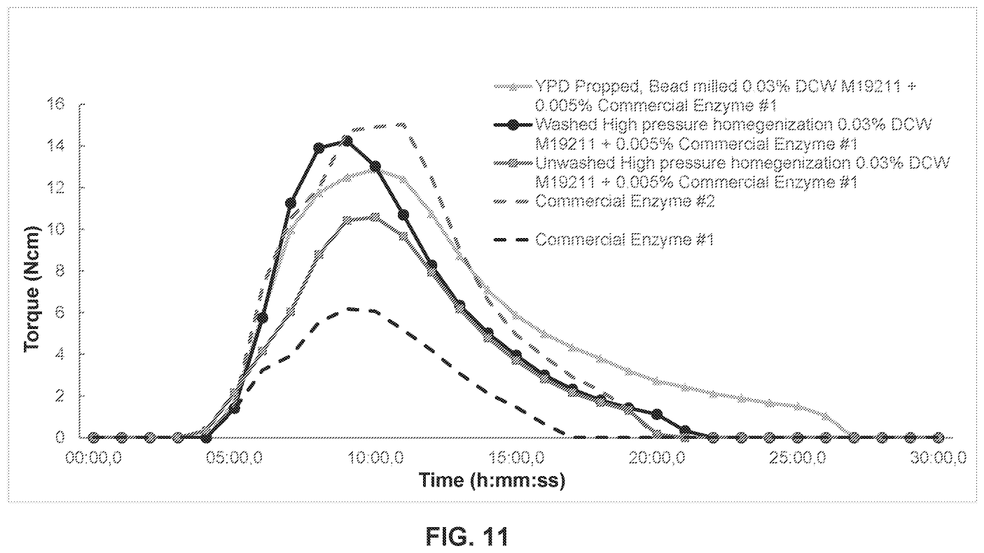

As seen in FIG. 11, the addition of the M19211 amylase-expressing yeast in combination with the 0.005% commercial alpha-amylase enzyme provided similar viscosity curves to the full 0.02% dose of two separate commercial alpha-amylase enzymes. The viscosity was indirectly measured using IKA Microstar30 overhead mixers which monitor torque trends, which increased as the viscosity increased. Based on previous experiments, the 0.005% commercial alpha-amylase enzyme addition did not successfully hydrolyze the corn and maxed out the machine's torque measuring capabilities at 30 Ncm and therefore was not included in this experiment. This data indicated that the disrupted M19211 yeast products were capable of eliminating nearly 75% of the commercial alpha-amylase enzyme dose.

TABLE 2
Description of M19211 strain.

| | | | Copies of | | | | | |
|---|---|---|---|---|---|---|---|---|
| | | | heterologous | | | | | |
| | Heterologous | | enzyme | | | | | |
| Strain | enzyme | Strain | integrated per | | | Signal | | |
| Name | expressed | background | chromosome | Promoter | Terminator | peptide | Linker | Tether |
| M19211 | P. furiosus | M16449 | 2 | ADH1 | DIT1 | S. cerevisiae | SEQ ID | SPI1 |
| | alpha-amylase | | | TEF1 | IDP1 | invertase | NO: 77 | SEQ ID |
| | SEQ ID NO: 64 | | | | | SEQ ID | | NO: 19 |
| | | | | | | NO: 39 | | |
| | T. hydrothermalis | | 4 | ADH1 | DIT1 | S. cerevisiae | SEQ ID | CCW12 |
| | alpha-amylase | | | TDH1 | IDP1 | α-mating | NO: 38 | SEQ ID |
| | SEQ ID NO: 63 | | | ADH1 | DIT1 | factor | | NO: 78 |
| | | | | TDH1 | IDP1 | SEQ ID | | |
| | | | | | | NO: 76 | | |

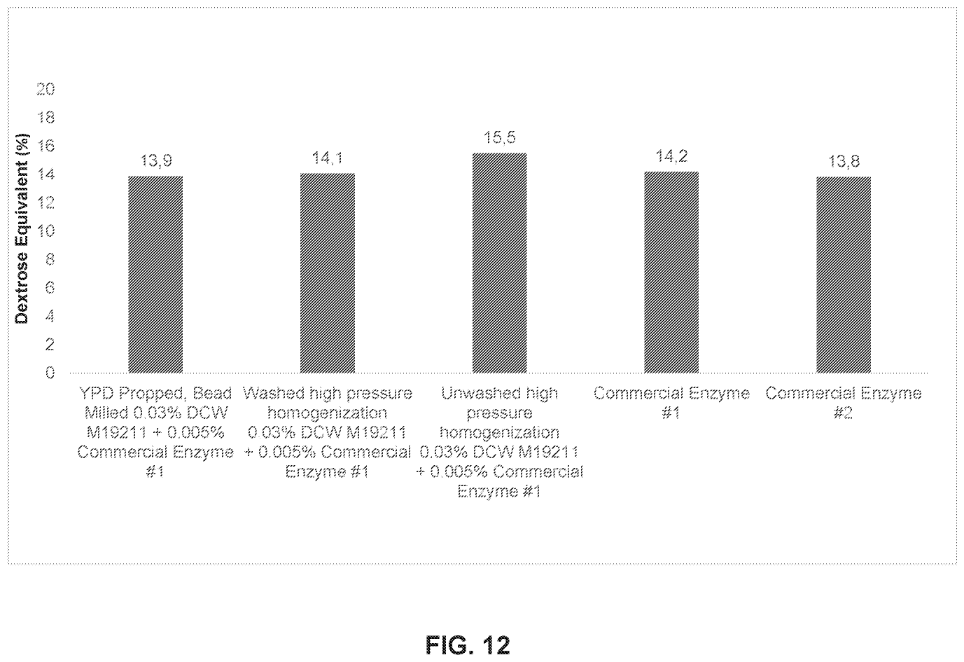

The subsequent liquefactions were evaluated for hydrolysis by measuring the dextrose equivalent. Samples were evaluated for solubilized reducing sugar concentrations using the DNS assay and correlated to glucose concentrations using a glucose standard curve. The % DE is a measure of the amount of reducing sugars and expressed as a percentage on a dry basis relative to dextrose. The dextrose equivalent gives an indication of the average degree starch hydrolysis. As seen in FIG. 12, each of the amylase-yeast liquefactions provided equivalent or higher % DE when compared to the commercial alpha-amylase enzyme 100% doses, indicating sufficient hydrolysis during the 2 h liquefaction.

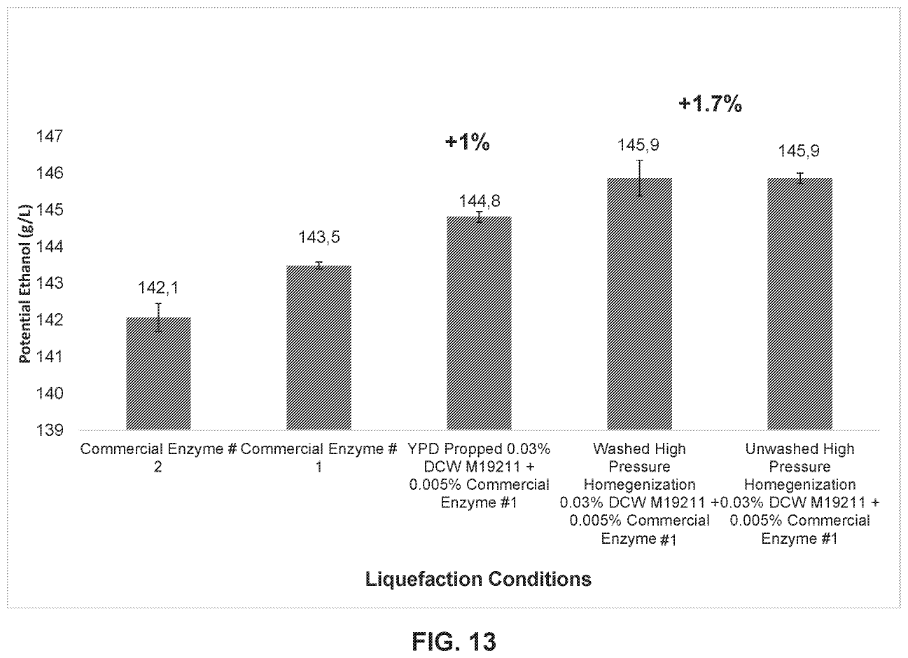

The liquefactions were subsequently fermented by adjusting the solids to 33% and fermented with the M2390 strain. The YPD-propped M19211 liquefaction provided a 1% potential ethanol yield increase relative to the 100% commercial alpha-amylase enzyme condition (Commercial alpha-amylase enzyme #1) and the disrupted M19211 cream products provided an additional 0.7% ethanol increase to the YPD propped cells, with an overall 1.7% potential ethanol increase compared to the enzyme control (FIG. 13).

### EXAMPLE IV—YIELD IMPROVEMENTS IN FERMENTATION WITH ADDITIONS OF YEAST EXTRACT DERIVED FROM YEAST STRAINS EXPRESSING VARIOUS ENZYMES

Nutrient rich commercial mash. Fermentations were performed using nutrient rich commercial mash collected from the field. The solids were lowered to 32% and fermentations performed in 200 mL bottles using 50 g of mash. Each fermentation received the same doses of 300 ppm urea, 0.6 AGU/gram total solids commercial glucoamylase (except for two of the GA yeast additions which received a 75% GA dose), and 0.05 g/L inoculum of the conventional strain M2390. Additionally, yeast expressing various amylolytic and yield enhancing enzymes (see a description in Table 3) were grown overnight in YPD at 35° C., centrifuged and resuspended in spent supernatant to equilibrate all of the dry cell weights. A total of 1 mL of each sample was bead beaten using glass beads in an MP Bio benchtop homogenizer to inactivate and disrupt the cells. The inactivated yeast was dosed into the respective fermentations at 0.1 g/L. Additionally, the parent M2390 strain was also bead beaten and dosed at the same concentration along with a water control to show both the effect of the yeast addition and the effect of the enzyme. The fermentations were mixed at 150 rpm and incubated at 33° C. for 24 h and the temperatures dropped to 31° C. for the remainder of the fermentation. Samples were collected after 54 h and the ethanol, glycerol, and glucose quantified using high performance liquid chromatography (HPLC).

TABLE 3
Description of the strains used in this example.

| Strain | Heterologous enzyme | Heterologous | Amino acid |
|---|---|---|---|
| Name | origin | enzyme expressed | sequence |
| M2390 | N/A | N/A | N/A |
| (control) | | | |
| M15035 | S. fibuligera | glucoamylase | SEQ ID NO: 3 |
| M15621 | R. emersonii | glucoamylase | SEQ ID NO: 67 |
| M14845 | G. stereothermophilus | maltogenic | SEQ ID NO: 2 |
| | | alpha-amylase | |
| M19211 | P. furiosus | thermostable | SEQ ID NO: 65 |
| | | alpha-amylase | |
| | T. hydrothermalis | thermostable | SEQ ID NO: 66 |
| | | alpha-amylase | |
| M10077 | S. fibuligera | alpha-amylase | SEQ ID NO: 68 |
| M17188 | B. amyloliquefaciens | alpha-amylase | SEQ ID NO: 69 |
| M11313 | C. brakii | phytase | SEQ ID NO: 73 |
| M10885 | S. fibuligera | protease | SEQ ID NO: 74 |
| M10890 | A. fumigatus | protease | SEQ ID NO: 75 |
| M11245 | A. fumigatus | trehalase | SEQ ID NO: 70 |
| M16283 | N. crassa | trehalase | SEQ ID NO: 71 |
| M5791 | A. niger | xylanase | SEQ ID NO: 72 |

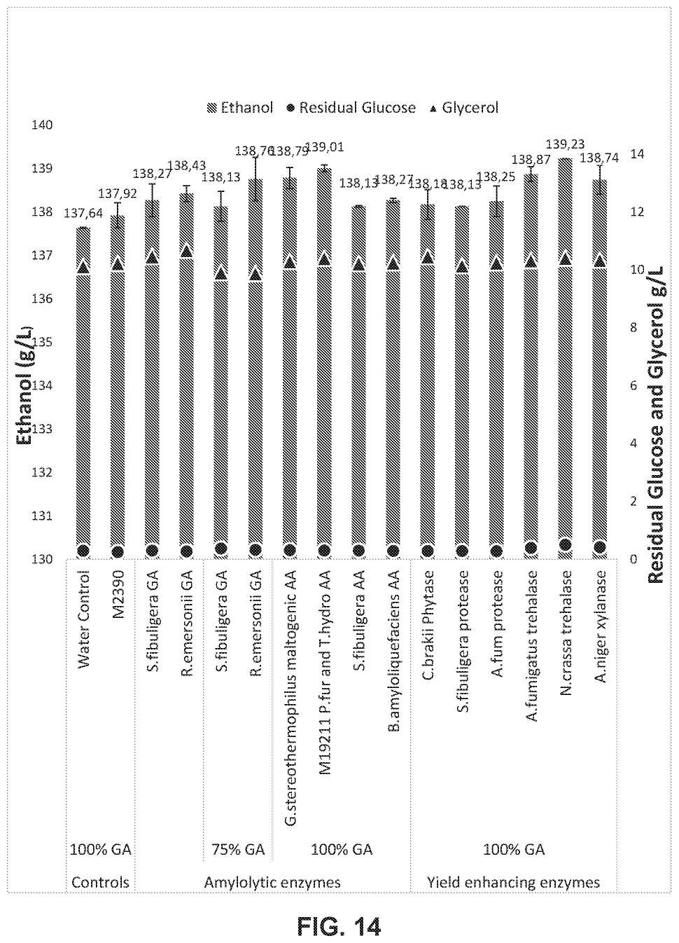

As seen in FIG. 14, the inactivated M2390 yeast addition provided a slight increase in ethanol production whereas the addition of most of the inactivated yeast enzyme strains provided an additional yield increase in the nutrient rich mash. Both of the glucoamylase (GA) strains expressing either the S. fibuligera or R. emersonii GA provided approximately a 0.5% yield increase over the water control condition with a 100% GA addition and enabled a 25% exogenous GA reduction using the 75% GA inclusion. The addition of alpha-amylase yeast provided a similar 0.36-1% yield increase compared to the water control condition, most notably the inactivated aforementioned M19211 strain expressing the tethered thermostable alpha-amylases provided one of the highest yield improvements with an additional 1% the water control. Each of the yield enhancing yeast additions provided >0.36% yield increase with the two separate trehalases from N. crassa and A. fumigatus and providing 0.9 and 1.16% yield improvements with a measureable decrease in residual DP2 and DP3's. The use of a cellulose expressing strain (xylanase from A. niger) was also successful in improving yields with a 0.8% yield increase. A summary of the yield improvements can be found in Table 4.

TABLE 4
Summary of the yield increases observed in
the fermentations presented in FIG. 14.

| | | % Yield Increase | |
|---|---|---|---|
| | | Relative to: | |
| GA | | Water | |
| Dose | Inactivated yeast addition | control | M2390 |
| 100% | M2390 | 0.21 | |
| GA | S. fibuligera GA expressing strain | 0.46 | 0.25 |
| | R. emersonii GA expressing strain | 0.57 | 0.37 |
| 75% | S. fibuligera GA expressing strain | 0.36 | 0.15 |
| GA | R. emersonii GA expressing strain | 0.82 | 0.61 |
| 100% | G. stereothermophilus maltogenic AA | 0.84 | 0.63 |
| GA | expressing strain | | |
| | M19211 (P. furiosus and | 1.00 | 0.79 |
| | T. hydrothermalis) AA expressing strain | | |
| | S. fibuligera AA expressing strain | 0.36 | 0.15 |
| | B. amyloliquefaciens AA expressing strain | 0.46 | 0.25 |
| | C. brakii phytase expressing strain | 0.39 | 0.18 |
| | S. fibuligera protease expressing strain | 0.36 | 0.15 |
| | A. fumigatus protease expressing strain | 0.45 | 0.24 |
| | A. fumigatus trehalase expressing strain | 0.90 | 0.69 |
| | N. crassa trehalase expressing strain | 1.16 | 0.95 |
| | A. niger xylanase expressing strain | 0.80 | 0.59 |

Nutrient poor commercial mash. Fermentations were performed using nutrient poor commercial mash collected from the field. The solids were lowered to 30% and fermentations performed in 100 mL serum bottles using 25 g of mash. Each fermentation received the same doses of 300 ppm urea, 0.6 AGU/gram total solids commercial glucoamylase and 0.05 g/L inoculum of the M2390 strain. Additionally, yeast expressing various amylolytic and yield enhancing enzymes were grown overnight in YPD at 35° C., centrifuged and resuspended in spent supernatant to equilibrate all of the dry cell weights. A total of 1 mL of each sample was bead beaten using glass beads in an MP Bio benchtop homogenizer to inactivate and disrupt the cells. The inactivated yeast was dosed into the respective fermentations at 0.1 g/L. Additionally, the parent M2390 strain was also bead beaten and dosed at the same concentration along with a water control to show both the effect of the yeast addition and the effect of the enzyme. The fermentations were mixed at 150 rpm and incubated at 33° C. for 24 h and the temperatures dropped to 31° C. for the remainder of the fermentation. Samples were collected after 54 h and the ethanol, glycerol, and glucose quantified using high performance liquid chromatography (HPLC).

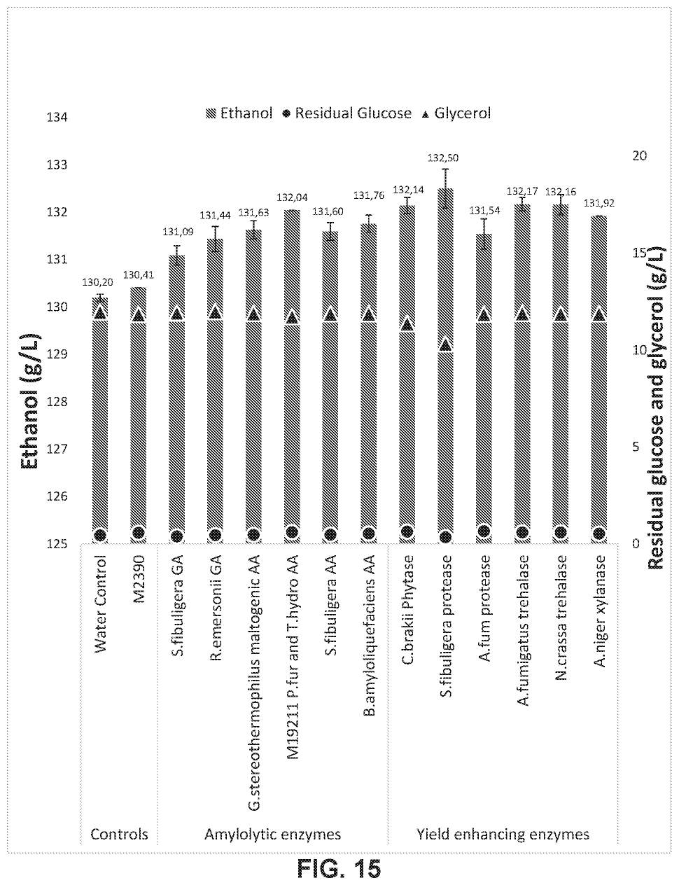

As seen in FIG. 15, the inactivated M2390 yeast addition provided a modest increase in ethanol production whereas the addition of most of the inactivated yeast enzyme strains provided an additional yield increase in the nutrient poor mash when compared to the water control. Both of the glucoamylase strains expressing either the S. fibuligera or R. emersonii GA provided approximately a 0.69-0.95% yield increase over the water control condition with a 100% GA. The addition of alpha-amylase yeast provided a similar 1.1-1.4% yield increase compared to the water control condition, most notably the inactivated aforementioned M19211 strain expressing the tethered thermostable alpha-amylases provided one of the highest yield improvements with an additional 1.42% over the water control. Each of the yield enhancing yeast additions provided >1% yield increase with the two separate trehalases from N. crassa and A. fumigatus and providing a 1.5% yield increase with a measureable decrease in residual carbohydrates having a degree of polymerization of 2 or 3 (DP2 and DP3, maltose and maltotriose). The protease strains each provided improvements, with the S. fibuligera protease providing the highest overall titer with a subsequent glycerol reduction. The addition of the phytase yeast also improved yield 1.5%. The use of a cellulose expressing strain (xylanase from A. niger) was also successful in improving yields with a 1.3% yield increase. A summary of the yield improvements can be found in Table 5.

TABLE 5
Summary of the yield increases observed
in fermentations presented in FIG. 15.

% Yield Increase
Relative to:

| | Water | |
|---|---|---|
| Strain | control | M2390 |
| M2390 | 0.17 | |
| S. fibuligera GA expressing strain | 0.69 | 0.52 |
| R. emersonii GA expressing strain | 0.95 | 0.79 |
| G. stereothermophilus maltogenic AA | 1.10 | 0.94 |
| expressing strain | | |
| M19211 (P. furiosus and | 1.42 | 1.25 |
| T. hydrothermalis AA expressing strain) | | |
| S. fibuligera AA expressing strain | 1.08 | 0.91 |
| B. amyloliquefaciens AA expressing strain | 1.20 | 1.03 |
| C. brakii phytase expressing strain | 1.49 | 1.33 |
| S. fibuligera protease expressing strain | 1.77 | 1.60 |
| A. fumigatus protease expressing strain | 1.03 | 0.87 |
| A. fumigatus trehalase expressing strain | 1.52 | 1.35 |
| N. crassa trehalase expressing strain | 1.51 | 1.34 |
| A. niger xylanase expressing strain | 1.32 | 1.16 |

### EXAMPLE V—COMPARISON OF DIFFERENT CELL DISRUPTION METHODS FOR INACTIVATING ALPHA-AMYLASE EXPRESSING YEAST FOR ADDITION IN LIQUEFACTIONS

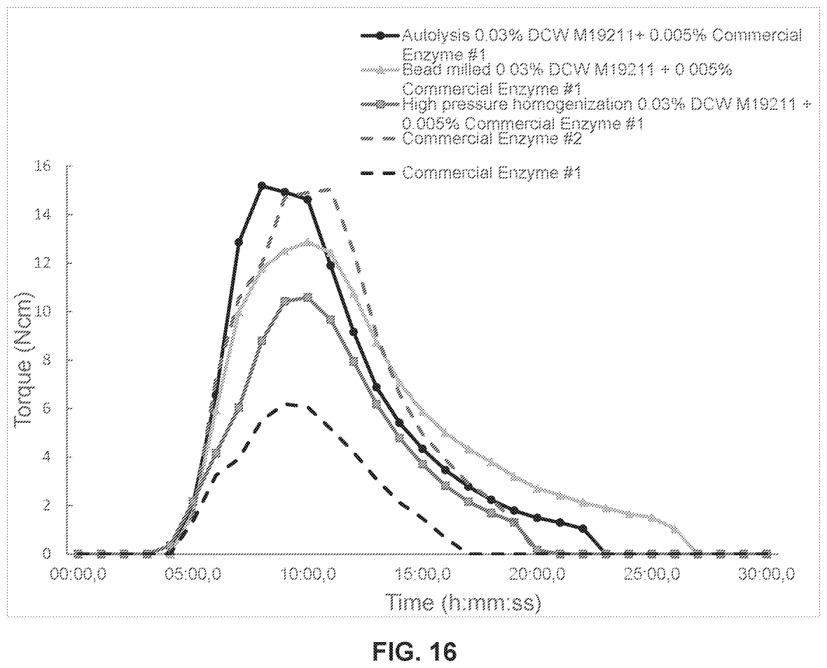

A similar lab-scale liquefaction as described previously was performed with the M19211 strain using various methods of inactivating the yeast. The yeast was prepared by either YPD propping overnight, or via a cream yeast production using molasses. The cream yeast concentrated to 20% solids in spent beer. The cream samples were disrupted using a high pressure homogenizer between 1000 and 1500 bar. The YPD propped culture were concentrated in spent supernatant and either bead beaten for 3 min using the benchtop homogenizer, or autolysized at 70° C. for 24 h. The disrupted cultures were each dosed at 0.03% grams DCW per grams of corn solids along with a 25% dose of commercial alpha-amylase enzyme (0.005% weight of enzyme per weight corn solids). As seen in FIG. 16, the addition of the M19211 amylase-expressing yeast with a 0.005% commercial alpha-amylase enzyme provided similar viscosity curves to the full 0.02% dose of two separate commercial alpha-amylase enzymes, representing commercially relevant conditions and variations with enzyme products. The changes in viscosity is indirectly measured using IKA Microstar30 overhead mixers which monitor torque trends, which increases as the viscosity increases, and Labworldsoft software. Based on previous experiments, the 0.005% commercial alpha-amylase enzyme addition does not successfully hydrolyze the corn and maxes out the machine's torque measuring capabilities at 30 Ncm and therefore was not included in this experiment. This data indicates that the disrupted M19211 cultures are capable of eliminating nearly 75% of the commercial alpha-amylase enzyme dose.

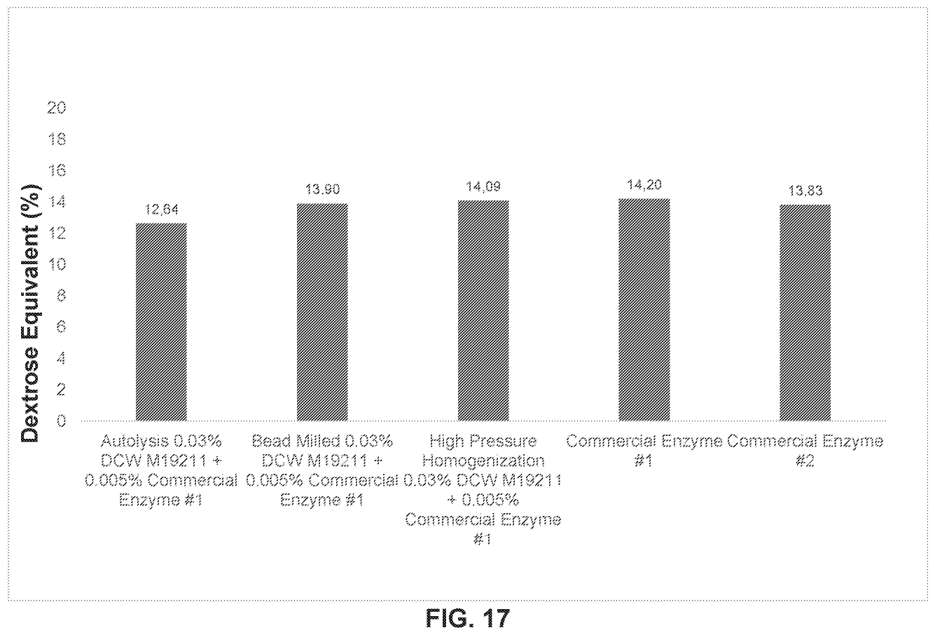

The subsequent liquefactions were evaluated for hydrolysis by measuring the dextrose equivalent. As seen in FIG. 17, each of the amylase-yeast liquefactions provided equivalent % DE when compared to the commercial 100% enzyme doses, indicating sufficient hydrolysis during the 2 h liquefaction.

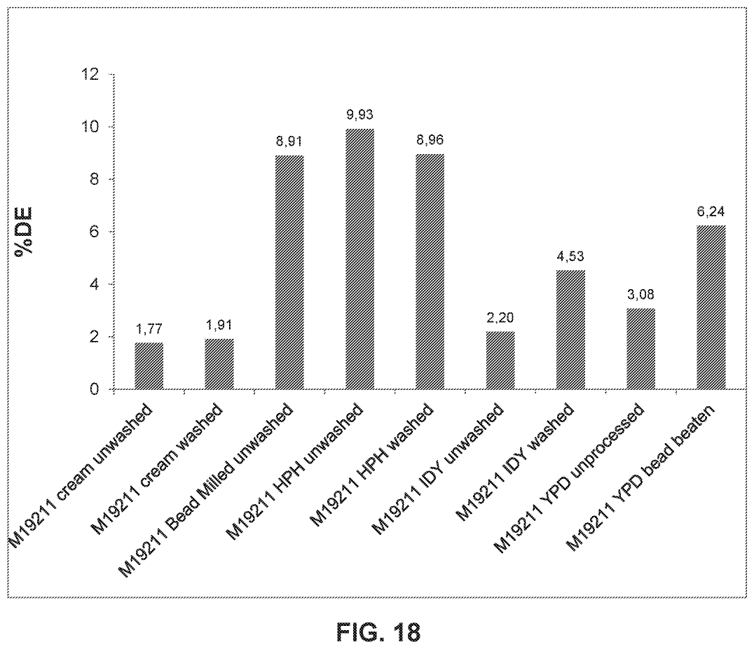

Strain M19211 was also evaluated for additional methods of processing to demonstrate potential product formats. The strain was either produced in a cream production using molasses in which the resulting cream yeast was either washed with water and resuspended to approximately 20% total DCW with water, or not washed and resuspended to 20% solids in spent beer. Both the washed and unwashed cream samples were disrupted using a high pressure homogenizer (HPH) between 1000 and 1500 bar. Both samples were also prepared into inactive dry yeast (IDY). All of these samples were compared to a YPD propped lab preparation in which the cells were either unprocessed or bead beaten for 3 mins as previously mentioned. All of the samples were compared to unprocessed cream or YPD grown cells to demonstrate an increase in activity post processing as the % DE was higher in a 1 gram mini-liquefaction (FIG. 18).

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Abernathy, D. G., Spedding, G., and Starcher, B. (2009). Analysis of Protein and Total Usable Nitrogen in Beer and Wine Using a Microwell Ninhydrin Assay. Journal of the Institute of Brewing 115, 122-127.

Pérez-Torrado R, Bruno-Bárcena J M, Matallana E. Monitoring stress-related genes during the process of biomass propagation of Saccharomyces cerevisiae strains used for wine making. Appl Environ Microbiol. 2005 November; 71(11):6831-7.

Praekelt U M, Meacock P A. MOL1, a Saccharomyces cerevisiae gene that is highly expressed in early stationary phase during growth on molasses. Yeast. 1992 September; 8(9):699-710.

Verduyn C, Postma E, Scheffers W A, Van Dijken J P. Effect of benzoic acid on metabolic fluxes in yeasts: a continuous-culture study on the regulation of respiration and alcoholic fermentation. Yeast. 1992 July; 8(7):501-17.

1. A process for improving the yield of a fermentation product made from a fermenting yeast cell in a fermenting medium, the process comprising:
(i) liquefying a liquefaction medium to obtain a fermentation medium; and/or
(ii) fermenting the fermentation medium with the fermenting yeast cell to obtain the fermentation product;
wherein the process further comprises including at least one of:
a first inactivated yeast product made from a first recombinant yeast host cell in the liquefaction medium and/or the fermentation medium, wherein the first recombinant yeast host cell comprises a first heterologous nucleic acid molecule for expressing a first heterologous enzyme and the first inactivated yeast product comprises the first heterologous enzyme;
a second recombinant yeast host cell in the liquefaction medium to obtain a second inactivated yeast product in the fermentation medium, wherein the second recombinant yeast host cell comprises a second heterologous nucleic acid molecule for expressing a second heterologous enzyme and the second inactivated yeast product comprises the second heterologous enzyme; and/or
a third inactivated yeast product made from a non-genetically modified yeast host cell to the liquefaction medium;
so as to improve the yield of the fermentation product.

2. The process of claim 1, wherein the first inactivated yeast product, the second inactivated yeast product and/or the third inactivated yeast product is a yeast extract.

3. The process of claim 2, further comprising:
bead milling, bead beating or high pressure homogenizing the first recombinant yeast host cell to obtain the first inactivated yeast product; or
bead milling, bead beating or high pressure homogenizing the non-genetically modified yeast host cell obtain the third inactivated yeast product.

4. (canceled)

5. The process of claim 1, wherein the second recombinant yeast host cell is provided as a cream yeast.

6. The process of claim 1, wherein the first heterologous nucleic acid molecule allows:
intracellular expression of the first heterologous enzyme;
expression of the first heterologous enzyme in association with a membrane of the first recombinant yeast host cell; or
expression of the first heterologous enzyme in a secreted form.

7. The process of claim 1, wherein the second heterologous nucleic acid molecule allows:
intracellular expression of the second heterologous enzyme;
expression of the second heterologous enzyme in association with a membrane of the second recombinant yeast host cell; or
expression of the second heterologous enzyme in a secreted form.

8. (canceled)

9. The process of claim 1, wherein the first heterologous nucleic acid molecule allows expression of the first heterologous enzyme tethered to a membrane of the first recombinant yeast host cell.

10. (canceled)

11. The process of claim 1, wherein the second heterologous second nucleic acid molecule allows expression of the second heterologous enzyme tethered to a membrane of the second recombinant yeast host cell.

12.-13. (canceled)

14. The process of claim 1, wherein the first heterologous nucleic acid molecule is operatively associated with a first promoter allowing expression of the first heterologous enzyme during propagation of the first recombinant yeast host cell and/or the second heterologous nucleic acid molecule is operatively associated with a second promoter allowing expression of the second heterologous enzyme during propagation of the second recombinant yeast host cell.

15. (canceled)

16. The process of claim 1, wherein the first heterologous enzyme and/or the second heterologous enzyme is an amylolytic enzyme, an esterase or a protease.

17. The process of claim 16, wherein the amylolytic enzyme has alpha-amylase activity, glucoamylase activity, trehalase activity, or xylanase activity; the esterase has phytase activity; and/or the protease has aspartic protease activity.

18. The process of claim 17, wherein:
the amylolytic enzyme having alpha-amylase activity comprises the amino acid sequence of any one of SEQ ID NO: 13, 60, 61, 62, 63, or 64; is a variant of the amino acid sequence of any one of SEQ ID NO: 13, 60, 61, 62, 63, or 64; or is a fragment of the amino acid sequence of any one of SEQ ID NO: 13, 60, 61, 62, 63, or 64;
the amylolytic enzyme having glucoamylase activity comprises the amino acid sequence of any one of SEQ ID NO: 3 or 67; is a variant of the amino acid sequence of any one of SEQ ID NO: 3 or 67; or is a fragment of the amino acid sequence of any one of SEQ ID NO: 3 or 67;
the amylolytic enzyme having trehalase activity comprises the amino acid sequence of SEQ ID NO: 70 or 71; is a variant of the amino acid sequence of SEQ ID NO: 70 or 71; or is a fragment of the amino acid sequence of SEQ ID NO: 70 or 71;
the amylolytic enzyme having xylanase activity comprises the amino acid sequence of SEQ ID NO: 72, is a variant of the amino acid sequence of SEQ ID NO: 72, or is a fragment of the amino acid sequence of SEQ ID NO: 72;
the esterase having phytase activity comprises the amino acid sequence of SEQ ID NO: 73, is a variant of the amino acid sequence of SEQ ID NO: 73, or is a fragment of the amino acid sequence of SEQ ID NO: 73; and/or
the protease having aspartic protease activity comprises the amino acid sequence of SEQ ID NO: 74 or 75; is a variant of the amino acid sequence of SEQ ID NO: 74 or 75; or is a fragment of the amino acid sequence of SEQ ID NO: 74 or 75.

19.-30. (canceled)

31. The process of claim 1, wherein the fermenting yeast cell is a fermenting recombinant yeast host cell.

32. The process of claim 31, wherein the fermenting recombinant yeast cell comprises:
a genetic modification for reducing the production of one or more native enzymes that function to produce glycerol or regulate glycerol synthesis,
a genetic modification for allowing the production of a second polypeptide having glucoamylase activity, and/or
a genetic modification for reducing the production of one or more native enzymes that function to catabolize formate.

33. (canceled)

34. The process of claim 1, wherein:
step (ii) is conducted under anaerobic conditions;
the fermenting medium comprises or is derived from corn, sugar cane or a lignocellulosic material; and/or
the fermentation product is ethanol.

35.-36. (canceled)

37. The process of claim 1, further comprising including an exogenous polypeptide having alpha-amylase activity with the third inactivated yeast product.

38.-66. (canceled)

67. A kit for improving yield of a fermentation product made from a fermenting yeast cell, the kit comprising (i) at least one component of a liquefaction medium and/or a fermentation medium for the fermenting yeast cell; and (ii) at least one of the first inactivated product, the second recombinant yeast host cell or the third inactivated product as defined in claim 1.

68.-70. (canceled)

71. A liquefaction medium comprising the first inactivated yeast product, the second recombinant yeast host cell, or the third inactivated yeast product as defined in claim 1.

72. A fermentation medium comprising the first inactivated yeast product, the second inactivated yeast product, or the third inactivated yeast product as defined in claim 1.
